(19) United States
(12) Patent Application Publication
(10) Pub. No.: US 2018/0117171 A1
(43) Pub. Date: May 3, 2018

(54) IMMUNOCONJUGATES FOR PROGRAMMING OR REPROGRAMMING OF CELLS

(71) Applicant: President and Fellows of Harvard College, Cambridge, MA (US)

(72) Inventors: David J. Mooney, Sudbury, MA (US); Roger Warren Sands, Chicago, IL (US); Joel Stern, Seaford, NY (US); Aileen W. Li, Norcross, GA (US); Rajiv Desai, San Diego, CA (US); Beverly Ying Lu, Alhambra, CA (US)

(21) Appl. No.: 15/563,878

(22) PCT Filed: Apr. 1, 2016
(86) PCT No.: PCT/US2016/025717

§ 371 Date: Oct. 2, 2017

Related U.S. Application Data
(60) Provisional application No. 62/145,053, filed on Apr. 9, 2015.
(60) Provisional application No. 62/141,684, filed on Apr. 1, 2015.

(51) Int. Cl. A61K 47/64; A61K 47/69; A61L 27/54; A61L 27/28; A61L 27/22; A61P 35/00; A61P 31/00; A61P 37/00; A61P 37/06; A61P 37/08; A61P 11/06; A61P 3/08; A61P 1/00; A61P 19/02; A61P 17/06; A61P 17/10; A61P 17/00; A61P 25/00; A61K 45/06; A61P 29/00

(52) U.S. Cl. CPC A61K 47/646; A61L 2430/02; A61L 27/54; A61L 27/28; A61L 27/227; A61P 35/00; A61P 31/00; A61P 37/00; A61P 37/06; A61P 37/08; A61P 11/06; A61P 3/08; A61P 1/00; A61P 19/02; A61P 17/06; A61P 17/10; A61P 17/00; A61P 25/00; A61K 45/06; A61P 29/00; A61L 2300/606; A61L 2300/416; A61L 2300/426; A61L 2300/43; A61L 2300/45; A61L 2300/432; A61K 47/6957

(57) ABSTRACT

The conjugate compositions and methods are useful to elicit/augment an immune response to a tumor or microbial infection or to reduce the severity of autoimmunity, chronic inflammation, allergy, asthma, periodontal disease, and transplant rejection.

### RELATED APPLICATIONS

This application claims the benefit of priority under 35 U.S.C. § 119(e) to U.S. Provisional Application No. 62/145,053, filed on Apr. 9, 2015 and U.S. Provisional Application No. 62/141,684, filed Apr. 1, 2015, each of which is incorporated herein by reference in its entirety.

### STATEMENT AS TO FEDERALLY SPONSORED RESEARCH

This invention was made with government support under 5R01DE019917-02, F30DK088518-03, and R01EB015498 awarded by the National Institutes of Health. The Government has certain rights in the invention.

### INCORPORATION-BY-REFERENCE OF SEQUENCE LISTING

The contents of the text file named “29297_116001WO_Sequence_Listing.txt”, which was created on Apr. 1, 2016 and is 12.2 KB in size, is hereby incorporated by reference in its entirety.

### FIELD OF THE INVENTION

The present invention relates to immune response modulation.

### BACKGROUND

Aberrant or misregulated immune responses are among the underlying mechanisms of numerous pathological conditions. Such conditions include cancers, autoimmune disorders, diseases of immunity, and conditions characterized by chronic inflammation.

Autoimmunity is a condition where the immune system mistakenly recognizes host tissue or cells as foreign. Autoimmune diseases affect millions of individuals worldwide. Common autoimmune disorders include type 1 diabetes mellitus, Crohn's disease, rheumatoid arthritis, and multiple sclerosis.

Chronic inflammation has been implicated in cancer, diabetes, depression, heart disease, stroke, Alzheimer's Disease, periodontitis, and many other pathologies. Aberrant or misregulated immune responses are also implicated in asthma and allergy, e.g., asthma is a prevalent disease with many allergen triggers.

Aberrant or pathological immune activation underlies diseases, such as autoimmune diseases, transplantation graft rejection, allergy, and asthma. These immune activation disorders are prevalent and contribute to significant morbidity and mortality. Few therapies exist that are sufficiently potent while maintaining specificity. Dendritic cells are cells of the immune system that connect the innate and adaptive immune system and are critical regulators of both immunity and tolerance. Dendritic cells play a central role as sentinels of the immune system that survey the environment and direct T cell responses both in health and disease. Pathologic T cell reactivity is a component of many diseases, including autoimmune diseases, such as diabetes mellitus and rheumatoid arthritis.

Few therapies exist to treat such diseases of the immune system, and those that do tend to have substantial side effects and rarely target the underlying mechanism of disease. Further, these agents often have pleiotropic effects, and due to their lack of specificity and narrow therapeutic windows, limited potencies. There is a need for effective prophylaxis and treatment of immune activation disorders with minimal or no side effects.

### SUMMARY OF THE INVENTION

The invention provides a solution to the long standing clinical problems of aberrant immune responses such as those involved in cancer immunity, autoimmunity, allergy/asthma, and chronic or inappropriate inflammation in the body, e.g., inflammation that leads to tissue/organ damage and destruction. In the context of cancer therapy, the challenge is how to treat cancer in view of a tumor's immune evasive phenotype. In the context of autoimmune disease, the challenge is how to dampen/inhibit a destructive immune response while preserving a productive immune response.

The compositions and methods direct the immune response of an individual to elicit an immune response to a tumor or away from a pathological or life-threatening immune response and toward a productive or non-damaging response.

Accordingly, an exemplary composition comprises an immunomodulatory agent covalently linked to an antigen and a delivery vehicle, wherein said antigen comprises a tumor antigen. For example, the adjuvant comprises a toll-like receptor (TLR) ligand such as a cytosine, guanine containing oligonucleotide. CpG oligodeoxynucleotides (or CpG ODN) are short single-stranded synthetic DNA molecules that contain a cytosine triphosphate deoxynucleotide (“C”) followed by a guanine triphosphate deoxynucleotide (“G”). The “p” refers to the phosphodiester link between consecutive nucleotides, although some ODN have a modified phosphorothioate (PS) backbone instead. In some embodiments, the CpG oligodeoxynucleotide is at least about 15, 16, 17, 18, 19, 20, 25, 26, 27, 28, 29, 30, 15-30, 20-30, 20-25, or more nucleotides long. When these CpG motifs are unmethylated, they act as immunostimulants or adjuvants. The CpG is recognized by TLR9 (i.e., CpG is a TLR9 ligand), which is constitutively expressed only in B cells and plasmacytoid dendritic cells (pDCs) in humans and other higher primates.

In various embodiments, the TLR ligand comprises a CpG oligonucleotide or a poly I:C poly nucleotide. Poly I:C is a mismatched double-stranded RNA with one strand being a polymer of inosinic acid, the other a polymer of cytidylic acid. Polyinosinic:polycytidylic acid (abbreviated poly I:C) is also an immunostimulant or adjuvant. In some embodiments, the polyI:C polynucleotide has a length of at least about, 0.1, 0.2, 0.3, 0.4, 0.5, 0.6, 0.7, 0.8, 0.9, 1, 1.2, 1.5, 2, 3, 4, 5, 6, 7, 8, 9, 1, 0.1-1, 0.2-1, 1-1.5, 0.5-1.5, 0.5-2, 1-5, 1.5-5, or 1.5-8 kilobases. In certain embodiments, the polyI:C polynucleotide has a length of about 0.1, 0.2, 0.3, 0.4, 0.5, 0.6, 0.7, 0.8, 0.9, 1, 1.2, 1.5, 2, 3, 4, 5, 6, 7, 8, 9, 1, 0.1-1, 0.2-1, 1-1.5, 0.5-1.5, 0.5-2, 1-5, 1.5-5, 1.5-8 or more kilobases. Optionally, it is used in the form of its sodium salt. Poly I:C interacts with TLR3 (i.e., poly I:C is a TLR 3 ligand), which is expressed in the membrane of B-cells, macrophages and dendritic cells. Optionally, CpG or poly I:C are condensed. For example, the adjuvant is condensed and then linked to an antigen; alternatively the adjuvant is linked to the antigen and then the conjugate is condensed. Exemplary condensing agents include poly-L-lysine (PLL), polyethylenimine (PEI), hexamine cobalt chloride, and TAT 47-57 peptide (YGRKKRRQRRR) (SEQ ID NO: 15).

The antigen to which an immunomodulatory agent is conjugated may be any antigen to which an immune response (or augmented immune response) or to which a tolerizing effect is desired. For example to elicit or augment an immune response, the tumor antigen comprises a tumor cell lysate. Exemplary tumor antigens and/or tumor lysate preparations to be used as antigens are described in U.S. Pat. No. 8,067,237, hereby incorporated by reference. For example, the antigen component of the conjuage comprises a central nervous system (CNS) cancer antigen, CNS Germ Cell tumor antigen, lung cancer antigen, Leukemia antigen, Multiple Myeloma antigen, Renal Cancer antigen, Malignant Glioma antigen, Medulloblastoma antigen, breast cancer antigen, prostate cancer antigen, ovarian cancer antigen, or Melanoma antigen. Alternatively, the antigen is obtained from an infectious disease pathogen, e.g., a bacterium, virus, or fungus.

Aspects of the present invention relate to vaccinating against or treating a bacterial, viral, or fungal infection. In various embodiments, a delivery vehicle comprising an immunoconjugate is administered to a subject in need of vaccination or treatment against an infection. In some embodiments, the immunoconjugate comprises, e.g., an antigen from a pathogen conjugated (e.g., directly or via a linker or spacer) to an adjuvant. For example, a pathogen includes but is not limited to a fungus, a bacterium (e.g., Staphylococcus species, Staphylococcus aureus, Streptococcus species, Streptococcus pyogenes, Pseudomonas aeruginosa, Burkholderia cenocepacia, Mycobacterium species, Mycobacterium tuberculosis, Mycobacterium avium, Salmonella species, Salmonella typhi, Salmonella typhimurium, Neisseria species, Brucella species, Bordetella species, Borrelia species, Campylobacter species, Chlamydia species, Chlamydophila species, Clostrium species, Clostrium botulinum, Clostridium difficile, Clostridium tetani, Helicobacter species, Helicobacter pylori, Mycoplasma pneumonia, Corynebacterium species, Neisseria gonorrhoeae, Neisseria meningitidis, Enterococcus species, Escherichia species, Escherichia coli, Listeria species, Francisella species, Vibrio species, Vibrio cholera, Legionella species, or Yersinia pestis), a virus (e.g., adenovirus, Epstein-Barr virus, Hepatitis A virus, Hepatitis B virus, Hepatitis C virus, Herpes simplex virus type 1, 2, or 8, human immunodeficiency virus, influenza virus, measles, Mumps, human papillomavirus, poliovirus, rabies, respiratory syncytial virus, rubella virus, or varicella-zoster virus), a parasite or a protozoa (e.g., Entamoeba histolytica, Plasmodium, Giardia lamblia, Trypanosoma brucei, or a parasitic protozoa such as malaria-causing Plasmodium). For example, a pathogen antigen is derived from a pathogen cell or particle described herein.

Preferably, the antigen and the adjuvant are in close proximity to one another such that a single cell takes up both elements of the conjugate.

The invention provides a device comprising a porous polymeric structure composition, e.g., delivery scaffold or device, that includes a conjugate comprising a tumor antigen, and a toll-like receptor (TLR) agonist (as an immunomodulatory agent, e.g., adjuvant). For example, the device comprises a polymeric structure composition, a tumor antigen, and a combination of toll-like receptor (TLR) agonists, wherein the TLR agonist is selected from the group consisting of TLR1, TLR2, TLR3, TLR4, TLR5, TLR6, TLR7, TLR8, TLR9, TLR10, TLR11, TLR12, and TLR13. For example, the polymeric structure comprises poly (D,L-lactide-co-glycolide) (PLG). Exemplary TLR agonists include pathogen associated molecular patterns (PAMPs), e.g., an infection-mimicking composition such as a bacterially-derived immunomodulator. TLR agonists include nucleic acid or lipid compositions [e.g., monophosphoryl lipid A (MPLA)].

Certain nucleic acids function as TLR agonists, e.g., TLR1 agonists, TLR2 agonists, TLR3 agonists, TLR4 agonists, TLR5 agonists, TLR6 agonists, TLR7 agonists, TLR8 agonists, TLR9 agonists, TLR10 agonists, TLR11 agonists, TLR12 agonists, or TLR13 agonists. In one example, the TLR agonist comprises a TLR9 agonist such as a cytosine-guanosine oligonucleotide (CpG-ODN), a poly(ethylenimine) (PEI)-condensed oligonucleotide (ODN) such as PEI-CpG-ODN, or double stranded deoxyribonucleic acid (DNA). TLR9 agonists are useful to stimulate plasmacytoid DCs. In another example, the TLR agonist comprises a TLR3 agonist such as polyinosine-polycytidylic acid (poly I:C), PEI-poly (I:C), polyadenylic-polyuridylic acid (poly (A:U)), PEI-poly (A:U), or double stranded ribonucleic acid (RNA).

TLR3 agonists are useful to stimulate CD8+ DCs in mice and CD141+ DCs in humans. A plurality of TLR agonists, e.g, a TLR3 agonist such as poly I:C and a TLR9 agonist such as CpG act in synergy to activate an anti-tumor immune response. For example, the device comprises a TLR3 agonist such as poly (I:C) and the TLR9 agonist (CpG-ODN) or a PEI-CpG-ODN. Preferably, the TLR agonist comprises the TLR3 agonist, poly (I:C) and the TLR9 agonist, CpG-ODN. The combination of poly (I:C) and CpG-ODN act synergistically as compared to the vaccines incorporating CpG-ODN or P(I:C) alone.

In some cases, the TLR agonist comprises a TLR4 agonist selected from the group consisting of lipopolysaccharide (LPS), MPLA, a heat shock protein, fibrinogen, heparin sulfate or a fragment thereof, hyaluronic acid or a fragment thereof, nickel, an opoid, al-acid glycoprotein (AGP), RC-529, murine β-defensin 2, and complete Freund's adjuvant (CFA). In other cases, the TLR agonist comprises a TLR5 agonist, wherein the TLR5 agonist is flagellin. Other suitable TLR agonists include TRL7 agonists selected from the group consisting of single-stranded RNA, guanosine anologs, irnidazoqinolines, and loxorbine. Additional TLR ligands/agonists and adjuvants are described in U.S. Patent Publication 20130202707; hereby incorporated by reference.

Aspects of the present subject matter relate to immunoconjugates comprising an antigen covalently linked to a Stimulator of Interferon Gene (STING) ligand (e.g., directly or via a linker or spacer). Non-limiting examples of STING ligands include cyclic dinucleotides such as cyclic guanosine monophosphate-adenosine (cGAMP), cyclic diadenylate monophosphate (c-di-AMP), and cyclic diguanylate monophosphate (c-di-GMP). Additional non-limiting examples of STING ligands are described in PCT International Patent Application Publication No. WO 2015/077354, published May 28, 2015; U.S. Pat. No. 7,709,458, issued May 4, 2010; U.S. Pat. No. 7,592,326, issued Sep. 22, 2009; and U.S. Patent Application Publication No. 2014/0205653, published Jun. 19, 2014, the entire contents of each of which are hereby incorporated herein by reference. In some embodiments, the cyclic dinucleotide is a compound comprising a 2′-5′ and/or 3′-5′ phosphodiester linkage between two purine (e.g., adenine and/or guanine) nucleotides.

In preferred embodiments, the antigen and the adjuvant or other immunomodulatory agent are covalently linked. For example, the immunomodulatory agent is covalently linked to the antigen by a carbamate bond, an ester bond, an amide bond, a triazole ring, a disulfide bond (such as between two cysteines), or a linker. Exemplary conjugates include antigen and adjuvant that are linked via a bifunctional maleimide (amine-sulfhydryl), carbodiimide (amine-carboxylic acid) or photo-click (norbornene-thiol) linker.

The material device or scaffold comprises poly(d,l-lactide-co-glycolide) (PLG) polymer, a cryogel (described in, e.g. U.S. Patent Application Publication No. 2014/0112990, published Apr. 24, 2014; hereby incorporated by reference), or a mesoporous silica (described in, e.g. U.S. Patent Application Publication No. 2015/0072009, published Mar. 12, 2015) composition. Exemplary compositions for such support structures include PLG polymers or other exemplary delivery vehicle or scaffold compositions such as polylactic acid, polyglycolic acid, PLGA polymers, alginates and alginate derivatives, gelatin, collagen, fibrin, hyaluronic acid, laminin rich gels, agarose, natural and synthetic polysaccharides, polyamino acids, polypeptides, polyesters, polyanhydrides, polyphosphazines, poly(vinyl alcohols), poly(alkylene oxides), poly(allylamines)(PAM), poly(acrylates), modified styrene polymers, pluronic polyols, polyoxamers, poly(uronic acids), poly(vinylpyrrolidone) and copolymers or graft copolymers cryogel delivery scaffolds/vehicles, or mesoporous silica delivery scaffolds.

A method of eliciting an anti-tumor immune response comprising administering to a subject the tumor antigen/adjuvant conjugate composition described above.

The compositions and methods direct the immune response of an individual away from a pathological or life-threatening response and toward a productive or non-damaging response. Dendritic cells (DCs) play a major role in protecting against autoimmune disease. Regulatory T cells (Treg) also play an important part in inhibiting harmful immunopathological responses directed against self or foreign antigens. The activities of these cell types are manipulated for the purpose of redirecting the immune response to provide a non-inflammatory and non-destructive state.

Provided herein is a composition comprising an antigen covalently linked to an immunomodulatory compound such as a tolerogen or an adjuvant. A covalent bond joins the two active molecules of the immunoconjugate. For example, the linkage comprises a zero length crosslinker (crosslinking is based on reaction between functional groups existing on the two active molecules of the conjugate) to something larger, e.g., when a crosslinking molecule (e.g., amino acid(s)) is used.

In the case of a tolerogenic conjugate, the antigen comprises a) a peptide associated with an immune activation disorder or b) a lysate of a cell associated with an immune activation disorder. In some embodiments, the tolerogen comprises a steroid such as dexamethasone prednisolone. In other embodiments, the tolerogen comprises vitamin D, retinoic acid, thymic stromal lymphopoietin, rapamycin, aspirin, transforming growth factor beta, interleukin-10, vasoactive intestinal peptide, vascular endothelial growth factor, retinoic acid, estrogen, anti-CTLA4 immunoglobulin, P-selectin, galectin 1, binding immunoglobulin protein (BiP), hepatocyte growth factor (HGF), immunoglobulin-like transcript 3 (ILT3), aspirin, resveratrol, rosiglitazone, curcumin, prednisolone, LF 15-0195, carvacrol, In some embodiments, the tolerogen comprises an apoptotic cell.

In some embodiments, an immunomodulatory agent comprises a mesoporous silica particle (e.g., a sphere or a rod), or structural material. Mesoporous silica has proinflammatory, e.g., adjuvant properties.

An antigen may be in the form of a protein, e.g., recombinant isolated protein; a polypeptide; or a peptide fragment. In some examples, the aberrant immune response is directed to a carbohydrate or glycoprotein. For example, an antigen includes an antibody or antibody fragment that targets a DC. In some cases, an antigen comprises a series of overlapping peptides sequences from a protein or polypeptide.

Exemplary immune activation disorders include an autoimmune disorder, an allergy, asthma, or transplant rejection.

In one embodiment, the immune activation disorder comprises an autoimmune disorder. For example, the autoimmune disorder comprises multiple sclerosis, type 1 diabetes mellitus, Crohn's disease, rheumatoid arthritis, systemic lupus erythematosus, scleroderma, alopecia areata, antiphospholipid antibody syndrome, autoimmune hepatitis, celiac disease, Graves' disease, Guillain-Barre syndrome, Hashimoto's disease, hemolytic anemia, idiopathic thrombocytopenic purpura, inflammatory bowel disease, ulcerative colitis, inflammatory myopathies, polymyositis, myasthenia gravis, primary biliary cirrhosis, psoriasis, Sjögren's syndrome, scleroderma, vasculitis, vitiligo, gout, atopic dermatitis, acne vulgaris, or autoimmune pancreatitis. An exemplary autoimmune disorder comprises type 1 diabetes. The peptide comprises a pancreatic peptide or protein. Exemplary pancreatic peptides or proteins include insulin, proinsulin, glutamic acid decarboxylase-65 (GAD65), insulinoma-associated protein 2, heat shock protein 60, ZnT8, islet-specific glucose-6-phosphatase catalytic subunit related protein (IGRP), or a fragment thereof. Exemplary peptides include B:9-23 (or 11-23) with the amino acid sequence, SHLVEALYLVCGERG (SEQ ID NO: 1); CP with the amino acid sequence, GLRILLLKV (SEQ ID NO: 2); Cl alternating D-, L-amino acids with the amino acid sequence, GLRILLLKV (SEQ ID NO: 2); and P277 residues 437-460 in the H-HSP65 sequence, VLGGGCALLRCIPALDSLTPANED (SEQ ID NO: 3).

In other cases, the autoimmune disorder comprises multiple sclerosis. For example, the peptide comprises myelin basic protein (MBP), myelin proteolipid protein, myelin-associated oligodendrocyte basic protein, myelin oligodendrocyte glycoprotein (MOG), or a fragment thereof. For example, the peptide comprises a fragment of MOG, e.g., MOG35-55, or MOG1-20. For example, the peptide comprises a fragment of MBP, e.g., MBP83-99, MBP85-99, MBP13-32, MBP111-129, MBP146-170. Additional exemplary peptides include random amino acid copolymers, e.g., Copolymer 1, a random amino acid copolymer of tyrosine (Y), glutamic acid (E), alanine (A), and lysine (K). Other example peptides include poly (Y, F, A, K) with the amino acid sequence, YFAK (SEQ ID NO: 4); poly (F, A, K) with the amino acid sequence, FAK; PLP139-151; J3 with the amino acid sequence, EKPKFEAYKAAAAPA (SEQ ID NO: 5); J5 with the amino acid sequence, EKPKVEAYKAAAAPA (SEQ ID NO: 6); and J2 with the amino acid sequence, EKPKYEAYKAAAAPA (SEQ ID NO: 7). In another example, the peptide is a myelin peptide, e.g., PLP139-154.

In some examples, the antigen comprises a citrullinated peptide, e.g., associated with rheumatoid arthritis.

A fragment of a protein or peptide described herein contains 1500 or less, 1250 of less, 1000 or less, 900 or less, 800 or less, 700 or less, 600 or less, 500 or less, 400 or less, 300 or less, 200, 100, 90, 80, 70, 60, 50, 40, 35, 30, 25, 20, 10, 8, 6, 4, or less amino acids.

Aspects of the present subject matter relate to immunoconjugates in which an antigen is conjugated, e.g., covalently linked, to an immunomodulatory agent, e.g. directly via a covalent bond or optionally via a linker or a spacer. Covalent bonds may have various lengths. Non-limiting examples of covalent bond lengths include lengths from about 1 angstrom to 3 angstroms. In various embodiments, the linker or spacer is sufficiently short as to promote the association of the antigen and the immunomodulatory agent conjugate with a single cell or to limit the association of the antigen and the immunomodulatory agent with a single cell. For example, the linker or spacer may be less than about 1, 2, 3, 4, 5, 6, 7, 8, 9, 10, 11, 12, 13, 14, 15, 20, 25, 30, 35, 40, 50, 1-5, 5-10, 5-15, 5-25, 10-30 or 5-50 angstroms long. Thus, in some embodiments, the antigen is no farther than 1, 2, 3, 4, 5, 6, 7, 8, 9, 10, 11, 12, 13, 14, 15, 20, 25, 30, 35, 40, 50, 1-5, 5-10, 5-15, 5-25, 10-30 or 5-50 from the immunomodulatory agent. In some embodiments, the antigen and immunomodulatory agent are directly linked via a covalent bond [without spacer linker compound(s)]. In certain embodiments, the linker or spacer is an amino acid, or a polypeptide comprising about 2, 3, 4, 5, 6, 7, 8, 9, or 10 amino acids. In some embodiments, the polypeptide comprises about 2, 3, 4, 5, 6, 7, 8, 9, or 10 glycines. Contacting a single cell with an immunoconjugate of the present subject matter reduces the off target effects that might result from delivering the antigen and the immunomodulatory agent to different cells.

In some embodiments, the tolerogen comprises dexamethasone or a derivative thereof. For example, the tolerogen comprises dexamethasone. In some examples, the tolerogen comprises dexamethasone derivatized with a phosphate at the primary alcohol on carbon 21. In some cases, the tolerogen is linked to the N-terminus of the peptide. For example, the antigen comprises a lysate, and e.g., the lysate comprises a peptide, where the tolerogen is linked to the N-terminus of the peptide. In other situations, the tolerogen is linked to the C-terminus or a peptide side chain. In some cases, the tolerogen is covalently linked to the antigen by a bond, e.g., a linker. Exemplary linkers include a carbodiimide linker, an amide linkage, and a carbamate bond. Additional coupling reactive chemistries can be employed to link the tolerogen to the antigen, e.g., NHS-esters (amine-amine), imidoesters (amine-amine), hydrazide (aldehyde-hydrazide), maleimides (sulfhydryl-sulfhydryl), azide alkyne Huisgen cycloaddition, and streptavidin-biotin conjugation, as well as click chemistries. In some cases, the linker is cleavable. For example, the linker is cleavable by enzymes, nucleophilic/basic reagents, reducing/oxidizing agents (e.g., inside a cell), photo-irradiation, thermal, electrophilic/acidic reagents, or organometallic/metal reagents.

In some embodiments, described herein is a composition comprising an antigen covalently linked to a tolerogen, where the antigen comprises a peptide associated with an immune activation disorder, where the peptide is derived from myelin oligodendrocyte glycoprotein (MOG), and where the tolerogen comprises dexamethasone or a derivative thereof. For example, the MOG is human MOG. In some cases, the peptide comprises amino acids 35-55 of human MOG. In other examples, the MOG is mouse MOG, e.g., with the amino acid sequence provided in GenBank No. Q61885.1, incorporated herein by reference. In some cases, the peptide comprises amino acids 35-55 of the mouse MOG with the amino acid sequence, MEVGWYRSPFSRVVHLYRNGK (SEQ ID NO: 8).

Aspects of the present subject matter provide delivery vehicles, and biomaterials comprising a recruitment composition. The recruitment composition is or contains a compound (or multiple compounds) that attracts a cell to and/or into the delivery vehicle or biomaterial.

Also provided is a delivery device comprising a composition described herein and a dendritic cell (DC) recruitment composition. For example, a delivery device is provided that comprises a dendritic cell (DC) recruitment composition and a composition comprising an antigen covalently linked to a tolerogen, where the antigen comprises a) a peptide associated with an immune activation disorder or b) a lysate of a cell associated with an immune activation disorder.

Exemplary DC recruitment compositions include granulocyte-macrophage colony stimulating factor (GM-CSF), FMS-like tyrosine kinase 3 ligand, N-formyl peptides, fractalkine, monocyte chemotactic protein-1, or macrophage inflammatory protein-3 (MIP-3α).

In some cases, the delivery device further comprises a Th1 promoting agent. For example, the Th1 promoting agent comprises a toll-like receptor (TLR) agonist. For example, the TLR agonist comprises a CpG oligonucleotide. In some examples, the Th1 promoting agent comprises a pathogen-associated molecular pattern (PAMP) composition or an alarmin. In some cases, the Th1 promoting agent comprises a TLR 3, 4, or 7 agonist.

In some embodiments, the delivery device comprises a microchip or a polymer. For example, the delivery device comprises a polymer. Example polymers include alginate, poly(ethylene glycol), hyaluronic acid, collagen, gelatin, poly (vinyl alcohol), fibrin, poly (glutamic acid), peptide amphiphiles, silk, fibronectin, chitin, poly(methyl methacrylate), poly(ethylene terephthalate), poly(dimethylsiloxane), poly(tetrafluoroethylene), polyethylene, polyurethane, poly(glycolic acid), poly(lactic acid), poly(caprolactone), poly(lactide-co-glycolide), polydioxanone, polyglyconate, BAK; poly(ortho ester I), poly(ortho ester) II, poly(ortho ester) III, poly(ortho ester) IV, polypropylene fumarate, poly[(carboxy phenoxy)propane-sebacic acid], poly[pyromellitylimidoalanine-co-1,6-bis(p-carboxy phenoxy)hexane], polyphosphazene, starch, cellulose, albumin, polyhydroxyalkanoates, Poly(lactide), and poly(glycolide).

In some cases, the polymer is hydrophobic or hydrophilic. For example, the polymer is hydrophobic. Suitable polymers include a polyanhydride or a poly (ortho ester).

Also provided is a method of reducing the severity of an autoimmune disorder in a subject in need thereof, comprising administering a composition or delivery device described herein to a subject suffering from an autoimmune disorder, where the tolerogen induces immune tolerance or a reduction in an immune response, and where the antigen is derived from a cell to which a pathologic autoimmune response associated with the autoimmune disorder is directed.

Examples of autoimmune disorders include multiple sclerosis, type 1 diabetes mellitus, Crohn's disease, rheumatoid arthritis, systemic lupus erythematosus, scleroderma, alopecia areata, antiphospholipid antibody syndrome, autoimmune hepatitis, celiac disease, Graves' disease, Guillain-Barre syndrome, Hashimoto's disease, hemolytic anemia, idiopathic thrombocytopenic purpura, inflammatory bowel disease, ulcerative colitis, inflammatory myopathies, polymyositis, myasthenia gravis, primary biliary cirrhosis, psoriasis, Sjögren's syndrome, vitiligo, gout, atopic dermatitis, acne vulgaris, and autoimmune pancreatitis.

In some examples, the tolerogenic vaccines are useful to put the “brakes on”, e.g., reduce the level of an immune response, in situations where it is beneficial to have an effective immunogenic response that then is subdued with this tolerogenic platform. Such a deliberate upregulation/downregulation of an immune response is analogous to being able to both use the brakes and gas pedal when driving to better control the immune response, e.g., regulation of an immune response in patients with sepsis. The tolerogenic compositions are useful to target compounds in the body are important to have but the level of which one would like to reduce, e.g. LDL microparticles, homocysteine, etc.

In one embodiment, the autoimmune disorder is multiple sclerosis. Also described is a method of reducing the severity of an allergy in a subject in need thereof, comprising administering a composition or delivery device described to a subject suffering from an allergy, where the antigen is associated with the allergy.

In one embodiment, the antigen comprises an allergen. Exemplary allergens include (Amb a 1 (ragweed allergen), Der p2 (Dermatophagoides pteronyssinus allergen, the main species of house dust mite and a major inducer of asthma), Betv 1 (major White Birch (Betula verrucosa) pollen antigen), Aln g I from Alnus glutinosa (alder), Api G I from Apium graveolens (celery), Car b I from Carpinus betulus (European hornbeam), Cor a I from Corylus avellana (European hazel), Mal d I from Malus domestica (apple), phospholipase A2 (bee venom), hyaluronidase (bee venom), allergen C (bee venom), Api m 6 (bee venom), Fel d 1 (cat), Fel d 4 (cat), Gal d 1 (egg), ovotransferrin (egg), lysozyme (egg), ovalbumin (egg), casein (milk) and whey proteins (alpha-lactalbumin and beta-lactaglobulin, milk), Ara h 1 through Ara h 8 (peanut), vicilin (tree nut), legumin (tree nut), 2S albumin (tree nut), profilins, heveins, lipid transfer proteins, Cor a 1 (hazelnut), Cor a 1.01 (hazel pollen), Cor a 1.02 (hazel pollen), Cor a 1.03 (hazel pollen), Cor a 1.04 (hazelnut), Bet v 1 (hazelnut), Cor a 2 (hazelnut), glycinin (soybean), Cor a 11 (hazelnut), Cor a 8 (tree nut), rJug r 1 (walnut), rJug r 2 (walnut), Jug r 3 (walnut), Jug r 4 (walnut), Ana o 1 (cashew nut), Ana o 2 (cashew nut), Cas s 5 (chestnut), Cas s 8 (chestnut), Ber e 1 (Brazil nut), Mal d 3 (apple), Pru p 3 (peach) and/or gluten.

A method of reducing the severity or frequency of an asthmatic attack in a subject in need thereof is provided, comprising administering a composition or delivery device described herein to a subject suffering from or at risk for an asthmatic attack, where the antigen provokes the asthmatic attack.

The method of claim 35, wherein the antigen comprises (Amb a 1 (ragweed allergen), Der p2 (Dermatophagoides pteronyssinus allergen, the main species of house dust mite and a major inducer of asthma), Betv 1 (major White Birch (Betula verrucosa) pollen antigen), Aln g I from Alnus glutinosa (alder), Api G I from Apium graveolens (celery), Car b I from Carpinus betulus (European hornbeam), Cor a I from Corylus avellana (European hazel), Mal d I from Malus domestica (apple), phospholipase A2 (bee venom), hyaluronidase (bee venom), allergen C (bee venom), Api m 6 (bee venom), Fel d 1 (cat), Fel d 4 (cat), Gal d 1 (egg), ovotransferrin (egg), lysozyme (egg), ovalbumin (egg), casein (milk) and whey proteins (alpha-lactalbumin and beta-lactaglobulin, milk), Ara h 1 through Ara h 8 (peanut), vicilin (tree nut), legumin (tree nut), 2S albumin (tree nut), profilins, heveins, lipid transfer proteins, Cor a 1 (hazelnut), Cor a 1.01 (hazel pollen), Cor a 1.02 (hazel pollen), Cor a 1.03 (hazel pollen), Cor a 1.04 (hazelnut), Bet v 1 (hazelnut), Cor a 2 (hazelnut), glycinin (soybean), Cor a 11 (hazelnut), Cor a 8 (tree nut), rJug r 1 (walnut), rJug r 2 (walnut), Jug r 3 (walnut), Jug r 4 (walnut), Ana o 1 (cashew nut), Ana o 2 (cashew nut), Cas s 5 (chestnut), Cas s 8 (chestnut), Ber e 1 (Brazil nut), Mal d 3 (apple), or Pru p 3 (peach).

A method is also provided for reducing transplant rejection in a subject in need thereof, comprising administering a composition or delivery device described herein to a subject prior to, during, or after a cell or tissue transplantation procedure, where the antigen comprises a molecule present in the transplanted cell but not present in the subject prior to the transplantation procedure.

For example, the antigen comprises an alloantigen. In some cases, the antigen comprises a minor or major histocompatibility antigen. For example, the antigen comprises a major histocompatibility complex (MHC) molecule, a HLA class I molecule, or a minor H antigen.

The antigen+tolerogen immunoconjugate composition is delivered to the body and leads to reprogramming of immune cells, thereby reducing the severity of autoimmune diseases or tissue destruction due to aberrant immune cell activation. Optionally, the antigen+tolerogen composition is associated with a delivery scaffold or vehicle.

In the latter case, the delivery scaffold composition comprises an antigen, a recruitment composition, and a tolerogen. This scaffold composition is useful for reduction of autoimmunity. The antigen is a purified composition (e.g., protein) or is a prepared cell lysate from cells to which an undesired immune response is directed. Exemplary recruitment compositions include granulocyte-macrophage colony stimulating factor (GM-CSF; AAA52578), FMS-like tyrosine kinase 3 ligand (AAA17999.1), N-formyl peptides, fractalkine (P78423), or monocyte chemotactic protein-1 (P13500.1). Exemplary tolerogens (i.e., agents that induce immune tolerance or a reduction in an immune response) include thymic stromal lymphopoietin (TSLP; Q969D9.1)), dexamethasone, vitamin D, retinoic acid, rapamycin, aspirin, transforming growth factor beta (P01137), interleukin-10 (P01137), vasoactive intestinal peptide (CAI21764), or vascular endothelial growth factor (AAL27435). The delivery vehicle scaffold optionally further comprises a Th1 promoting agent such as a toll-like receptor (TLR) agonist, e.g., a polynucleotide such as CpG. Th1 promoting agents are often characterized by pathogen-associated molecular patterns (PAMPs) or microbe-associated molecular patterns (MAMPs) or alarmins. PAMPs or MAMPs are molecules associated with groups of pathogens, that are recognized by cells of the innate immune system via TLRs. For example, bacterial Lipopolysaccharide (LPS), an endotoxin found on the gram negative bacterial cell membrane of a bacterium, is recognized by TLR 4. Other PAMPs include bacterial flagellin, lipoteichoic acid from Gram positive bacteria, peptidoglycan, and nucleic acid variants normally associated with viruses, such as double-stranded RNA (dsRNA) or unmethylated CpG motifs. Thus, additional exemplary Th1 promoting agents comprise a TLR 3, 4, or 7 agonist such as poly (I:C), LPS/MPLA (monophosphate lipid A), or imiquimod, respectively. CpG and/or poly I:C are optionally condensed, e.g., as described in application Ser. Nos. 12/867,426 and 13/741,271, each of which is incorporated by reference. Exemplary TLR ligands include the following compounds: TLR7 Ligands (human & mouse TLR7)-CL264 (Adenine analog), Gardiquimod™ (imidazoquinoline compound), Imiquimod (imidazoquinoline compound), and Loxoribine (guanosine analogue); TLR8 Ligands (human TLR8 & mouse TLR7)-Single-stranded RNAs; E. coli RNA; TLR7/8 Ligands—(human, mouse TLR7 & human TLR8)—CL075 (thiazoloquinoline compound), CL097 (water-soluble R848), imidazoquinoline compound, Poly(dT) (thymidine homopolymer phosphorothioate oligonucleotide (ODN)), and R848 (Imidazoquinoline compound).

Delivery device scaffolds for conjugates, e.g., antigen+immunomodulatory agent such as an adjuvant or antigen+tolerogen, are optionally delivered to bodily tissues in material devices such as poly(d,l-lactide-co-glycolide) (PLG) polymers or other exemplary delivery vehicle scaffold compositions such as polylactic acid, polyglycolic acid, PLGA polymers, alginates and alginate derivatives, gelatin, collagen, fibrin, hyaluronic acid, laminin rich gels, agarose, natural and synthetic polysaccharides, polyamino acids, polypeptides, polyesters, polyanhydrides, polyphosphazines, poly(vinyl alcohols), poly(alkylene oxides), poly(allylamines)(PAM), poly(acrylates), modified styrene polymers, pluronic polyols, polyoxamers, poly(uronic acids), poly(vinylpyrrolidone) and copolymers or graft copolymers of any of the above, e.g., as described in U.S. Pat. No. 8,067,237. For example, the delivery device scaffold composition includes an RGD-modified alginate. Other material devices include cryogel delivery scaffolds/vehicles, e.g., as described in U.S. Patent Application Publication No. 2014/0112990 and mesoporous silica delivery scaffolds/vehicles, e.g., as described in U.S. Patent Application Publication No. 2015/0072009.

The delivery vehicle scaffolds mediate sustained release of the factors loaded therein in a controlled spatio-temporal manner. For example, the factors are released over a period of days (e.g., 1, 2, 3, 4, 5, 7, 10, 12, 14 days or more) compared to bolus delivery (in the absence of a delivery scaffold/vehicle) of factors or antigens. Bolus delivery often leads to little or no effect due to short-term presentation in the body, adverse effects, or an undesirable immune response if very high doses are provided, whereas scaffold delivery avoids such events. Preferably, the delivery device scaffold is made from a non-inflammatory polymeric composition such as alginate, poly(ethylene glycol), hyaluronic acid, collagen, gelatin, poly (vinyl alcohol), fibrin, poly (glutamic acid), peptide amphiphiles, silk, fibronectin, chitin, poly(methyl methacrylate), poly(ethylene terephthalate), poly(dimethylsiloxane), poly(tetrafluoroethylene), polyethylene, polyurethane, poly(glycolic acid), poly(lactic acid), poly(caprolactone), poly(lactide-co-glycolide), polydioxanone, polyglyconate, BAK; poly(ortho ester I), poly(ortho ester) II, poly(ortho ester) III, poly(ortho ester) IV, polypropylene fumarate, poly[(carboxy phenoxy)propane-sebacic acid], poly[pyromellitylimidoalanine-co-1,6-bis(p-carboxy phenoxy)hexane], polyphosphazene, starch, cellulose, albumin, polyhydroxyalkanoates, or others known in the art (Polymers as Biomaterials for Tissue Engineering and Controlled Drug Delivery. Lakshmi S. Nair & Cato T. Laurencin, Adv Biochem Engin/Biotechnol (2006) 102: 47-90 DOI 10.1007/b137240). Alternatively, a polymeric composition that provides a low level of inflammation may also be useful, as it may aid in recruitment and/or activation of dendritic cells, particularly biasing the cells towards a Th1 response. Poly(lactide), poly(glycolide), their copolymers, and various other medical polymers may also be useful in this regard. Ceramic or metallic materials may also be utilized to present these factors in a controllable manner. For example, calcium phosphate materials are useful. In the context of bone, silica or other ceramics are also be useful.

In some examples, composite materials may be utilized. For example, immune activating factors (e.g., antigen, tolerogen, or Th1 promoting agent) are encapsulated in microspheres such as poly (lactide-co-glycolide) (PLG) microspheres, which are then dispersed in a hydrogel such as an alginate gel. Cells, e.g., DCs and/or Tregs, are recruited to or near the surface, or into the delivery vehicle scaffold, where they may reside for some period of time as they, are exposed to antigens and other factors described above, and then migrate away to bodily tissues such as lymph nodes, where they function to induce immune tolerance. Alternatively, the delivery vehicle scaffold with cells may create a mimic of a secondary lymphoid organ. Following contact with the loaded device scaffolds, such cells become activated to redirect the immune response from a Th1/Th2/Th17 response (autoimmunity and chronic inflammation) to a Treg response or from a pathogenic Th2 state toward a Th1 state (in the case of allergy/asthma). Directing the immune response away from a Th2 response and toward a Treg response leads to a clinical benefit in allergy, asthma. For autoimmunity, the therapeutic method is carried out by identifying a subject suffering from or at risk of developing an autoimmune disease and administering to the subject the loaded delivery device scaffolds (antigen (autoantigen)+recruitment composition+tolerogen), leading to an alteration in the immune response from a Th1/Th17 to T regulatory biased immune response. For allergy/asthma, the therapeutic method is carried out by identifying a subject suffering from or at risk of developing an allergic response or asthma and administering to the subject the loaded delivery vehicle scaffolds (antigen (allergen)+recruitment composition+adjuvant (Th1-promoting adjuvant)), thereby leading to an alteration in the immune response from a Th2 response to a Th1 biased immune response (allergy/asthma).

A method of preferentially directing a Th1-mediated antigen-specific immune response is therefore carried out by administering to a subject a delivery vehicle with a scaffold comprising an antigen, a recruitment composition and an adjuvant. A dendritic cell is recruited to the delivery device scaffold, exposed to antigen, and then migrates away from the delivery device scaffold into a tissue of the subject, having been educated/activated to preferentially generate a Th1 immune response compared to a pathogenic Th2 immune response based on the exposure. As a result, the immune response is effectively skewed or biased toward the Th1 pathway versus the Th2 pathway. Such a bias is detected by measuring the amount and level of cytokines locally or in a bodily fluid such as blood or serum from the subject. For example, a Th1 response is characterized by an increase in interferon-γ (IFN-gamma). As discussed above, the delivery device scaffold optionally also comprises a Th1 promoting agent.

The compositions and methods are suitable for treatment of human subjects; however, the compositions and methods are also applicable to companion animals such as dogs and cats as well as livestock such as cows, horses, sheep, goats, pigs.

The delivery vehicle scaffolds are useful to manipulate the immune system of an individual to treat a number of pathological conditions that are characterized by an aberrant, misdirected, or otherwise inappropriate immune response, e.g., one that causes tissue damage or destruction. Such conditions include autoimmune diseases. For example, a method of reducing the severity of an autoimmune disorder is carried out by identifying a subject suffering from an autoimmune disorder and administering to the subject a delivery vehicle scaffold composition comprising an antigen (e.g., a purified antigen or a processed cell lysate), a recruitment composition, and a tolerogen. Preferably, the antigen is derived from or associated with a cell to which a pathologic autoimmune response is directed. In one example, the autoimmune disorder is type 1 diabetes and the antigen comprises a pancreatic cell-associated peptide or protein antigen, e.g., insulin, proinsulin, glutamic acid decarboxylase-65 (GAD65), insulinoma-associated protein 2, heat shock protein 60, ZnT8, and islet-specific glucose-6-phosphatase catalytic subunit related protein or others as described in Anderson et al., Annual Review of Immunology, 2005. 23: p. 447-485; or Waldron-Lynch et al., Endocrinology and Metabolism Clinics of North America, 2009. 38(2): p. 303). In another example, the autoimmune disorder is multiple sclerosis and the peptide or protein antigen comprises myelin basic protein, myelin proteolipid protein, myelin-associated oligodendrocyte basic protein, and/or myelin oligodendrocyte glycoprotein. Additional examples of autoimmune diseases/conditions include Crohn's disease, rheumatoid arthritis, Systemic lupus erythematosus, Scleroderma, Alopecia areata, Antiphospholipid antibody syndrome, Autoimmune hepatitis, Celiac disease, Graves' disease, Guillain-Barre syndrome, Hashimoto's disease, Hemolytic anemia, Idiopathic thrombocytopenic purpura, inflammatory bowel disease, ulcerative colitis, inflammatory myopathies, Polymyositis, Myasthenia gravis, Primary biliary cirrhosis, Psoriasis, Sjögren's syndrome, Vitiligo, gout, celiac disease, atopic dermatitis, acne vulgaris, autoimmune hepatitis, and autoimmune pancreatitis.

The delivery vehicle scaffolds are also useful to treat or reduce the severity of other immune disorders such as a chronic inflammatory disorder or allergy/asthma. In this context, the method includes the steps of identifying a subject suffering from chronic inflammation or allergy/asthma and administering to the subject a delivery device scaffold composition comprising an antigen associated with that disorder, a recruitment composition, and an adjuvant. The vaccine is useful to reduce acute asthmic exacerbations or attacks by reducing/eliminating the pathogenic response to the allergies. In the case of allergy and asthma, the antigen comprises an allergen that provokes allergic symptoms, e.g., histamine release or anaphylaxis, in the subject or triggers an acute asthmatic attack. For example, the allergen comprises (Amb a 1 (ragweed allergen), Der p2 (Dermatophagoides pteronyssinus allergen, the main species of house dust mite and a major inducer of asthma), Betv 1 (major White Birch (Betula verrucosa) pollen antigen), Aln g I from Alnus glutinosa (alder), Api G I from Apium graveolens (celery), Car b I from Carpinus betulus (European hornbeam), Cor a I from Corylus avellana (European hazel), Mal d I from Malus domestica (apple), phospholipase A2 (bee venom), hyaluronidase (bee venom), allergen C (bee venom), Api m 6 (bee venom), Fel d 1 (cat), Fel d 4 (cat), Gal d 1 (egg), ovotransferrin (egg), lysozyme (egg), ovalbumin (egg), phleum pretense pollen (grass allergens; Phi p1 and Phi p 5); Api m 1 (bee venom allergen), casein (milk) and whey proteins (alpha-lactalbumin and beta-lactaglobulin, milk), and Ara h 1 through Ara h 8 (peanut). The compositions and methods are useful to reduce the severity of and treat numerous allergic conditions, e.g., latex allergy; allergy to ragweed, grass, tree pollen, and house dust mite; food allergy such as allergies to milk, eggs, peanuts, tree nuts (e.g., walnuts, almonds, cashews, pistachios, pecans), wheat, soy, fish, and shellfish; hay fever; as well as allergies to companion animals, insects, e.g., bee venom/bee sting or mosquito sting. Preferably, the antigen is not a tumor antigen or tumor lysate.

Also within the invention are vaccines comprising the loaded delivery device scaffold(s) described above and a pharmaceutically-acceptable excipient for injection or implantation into a subject for the to elicit antigen specific immune tolerance to reduce the severity of disease. Other routes of administration include topically affixing a skin patch comprising the delivery device scaffold or delivering scaffold compositions by aerosol into the lungs or nasal passages of an individual.

In addition to the conditions described above, the delivery vehicle scaffolds and systems are useful for treatment of periodontitis. One example of a biomaterial system for use in vivo that recruits dendritic cells and promotes their activation towards a non-inflammatory phenotype comprises a biomaterial matrix or scaffold, e.g., a hydrogel such as alginate, and a bioactive factor such as GM-CSF or thymic stromal lymphopoietin (TSLP) for use in dental or periodontal conditions such as periodontitis. Periodontitis is a destructive disease that affects the supporting structures of the teeth including the periodontal ligament, cementum, and alveolar bone. Periodontitis represents a chronic, mixed infection by gram-negative bacteria, such as Porphyromonas gingivalis, Prevotella intermedia, Bacteroides forsythus, Actinobacillus actinomycetemcomitans, and gram positive organisms, such as Peptostreptococcus micros and Streptococcus intermedius.

The methods address regulatory T-cell modulation of inflammation in periodontal disease. DCs can elicit anergy and apoptosis in effector cells in addition to inducing regulatory T cells. Other mechanisms include altering the balance between Th1, Th2, Th17 and T regs. For example, TSLP is known to enhance Th2 immunity and in addition to increasing T reg numbers could increase the Th2 response. The materials recruit and program large numbers of tolerogenic DCs to promote regulatory T-cell differentiation and mediate inflammation in rodent models of periodontitis. More specifically, the recruitment, appropriate activation, and migration to the lymph nodes of appropriately activated DCs leads to the formation of high numbers of regulatory T-cells, and decreased effector T-cells, reducing periodontal inflammation.

Another aspect of the present invention addresses the mediation of inflammation in concert with promotion of regeneration. In particular, plasmid DNA (pDNA) encoding BMP-2, delivered from the material system that suppresses inflammation, reduces inflammation via DC targeting and enhances the effectiveness of inductive approaches to regenerate alveolar bone in rodent models of periodontitis. For example, significant alveolar bone regeneration results from a material that first reduces inflammation, and then actively directs bone regeneration via induction of local BMP-2 expression.

The invention provides materials that function to modulate the inflammation-driven progression of periodontal disease, and then actively promote regeneration after successful suppression of inflammation. Moreover, the compositions and methods described herein can be translated readily into new materials for guided tissue regeneration (GTR). Unlike current GTR membranes that simply provide a physical barrier to cell movement, the new materials actively regulates local immune and tissue rebuilding cell populations in situ. More broadly, inflammation is a component of many other clinical challenges in dentistry and medicine, including Sjogren's and other autoimmune diseases, and some forms of temporomandibular joint disorders. The present invention has wide utility in treating many of these diseases characterized by inflammation-mediated tissue destruction. Further, the material systems also provide novel and useful tools for basic studies probing DC trafficking, activation, T-cell differentiation, and the relation between the immune system and inflammation. In addition to the conditions and diseases described above, the compositions and methods are also useful in wound healing, e.g., to treat smoldering wounds, thereby altering the immune system toward healing and resolution of the wound.

The compositions and methods described herein harness the tolerogenic potential of dendritic cells (DC) to develop more specific and potent therapies for immune activation disorders. In some cases, chemokines are used to recruit dendritic cells; in other cases, scaffolds are used without chemokines, as a means to provide sustained release/presentation of the antigen conjugate. The compositions and methods deliver antigens (e.g., autoantigens or allergens) to tolerize DC in situ. For example, using the methods described herein, the antigens are delivered to a sufficient number of DC to treat or reduce the severity of an immune activation disorder. Optionally, the compositions are provided in or on a material scaffold or device; in such cases, the scaffold also serves to recruit cells, e.g., even in the absence of additional factors such as chemokines. The invention is based in part on the discovery that a tolerogen covalently coupled to an antigen potently attenuates antigen-specific pathogenic T cell responses in vitro and in vivo compared to the uncoupled compounds.

The antigen portion of the immunoconjugate is presented by DC, and the immunoconjugate induces a tolerogenic phenotype in DC. Unlike many immunosuppressants that non-specifically dampen immunity or biologics that target DC but do not incorporate programming factors, the immunoconjugates described herein coordinate the presentation of antigen and programming factor in proximity to one another to generate tolerogenic dendritic cells that dampen both innate and adaptive immunity. For example, the antigen and tolerogen are covalently linked to each other, and thus are moieties are very close, e.g, molecular scale closeness. In some of the constructs, a glycine linker is used as a spacer. For example, the Dex mog compound optionally has a glycine (that functions as a spacer) in between the Dex and peptide. In another example, e.g., ovalbumin construct, OVA is directly linked the steroid. In both cases, and the constructs were effective to target individual cells to tolerize them to the antigen.

The constructs are sized such that a one single individual cell takes up and is functionally modified by both elements of the linked antigen+immunomodulatory agent, e.g., tolerogen or adjuvant. In the case of tolerogen constructs, the immunoconjugates elicit antigen specific T cell tolerance. The immunoconjugates are useful for treating/preventing diseases characterized by aberrant or undesired immune activation, e.g., autoimmune disease, allergy, asthma, and transplant rejection.

In accordance with any method described herein, a subject comprises a mammal, e.g., a human, dog, cat, cow, horse, sheep, goat, or pig. For example, the mammal is a human.

Polypeptides and other compositions used to load the scaffolds are purified or otherwise processed/altered from the state in which they naturally occur. For example, a substantially pure polypeptide, factor, or variant thereof is preferably obtained by expression of a recombinant nucleic acid encoding the polypeptide or by chemically synthesizing the protein. A polypeptide or protein is substantially pure when it is separated from those contaminants which accompany it in its natural state (proteins and other naturally-occurring organic molecules). Typically, the polypeptide is substantially pure when it constitutes at least 60%, by weight, of the protein in the preparation. Preferably, the protein in the preparation is at least 75%, more preferably at least 90%, and most preferably at least 99%, by weight. Purity is measured by any appropriate method, e.g., column chromatography, polyacrylamide gel electrophoresis, or HPLC analysis. Accordingly, substantially pure polypeptides include recombinant polypeptides derived from a eucaryote but produced in E. coli or another procaryote, or in a eucaryote other than that from which the polypeptide was originally derived.

In some situations, dendritic cells or other cells, e.g., immune cells such as macrophages, B cells, T cells, used in the methods are purified or isolated. With regard to cells, the term “isolated” means that the cell is substantially free of other cell types or cellular material with which it naturally occurs. For example, a sample of cells of a particular tissue type or phenotype is “substantially pure” when it is at least 60% of the cell population. Preferably, the preparation is at least 75%, more preferably at least 90%, and most preferably at least 99% or 100%, of the cell population. Purity is measured by any appropriate standard method, for example, by fluorescence-activated cell sorting (FACS). In other situations, cells are processed, e.g., disrupted/lysed and the lysate fractionated for use as an antigen in the delivery vehicle scaffold.

Polynucleotides, polypeptides, or other agents are purified and/or isolated. Specifically, as used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. Purified also defines a degree of sterility that is safe for administration to a human subject, e.g., lacking infectious or toxic agents.

By “isolated nucleic acid” is meant a nucleic acid that is free of the genes which flank it in the naturally-occurring genome of the organism from which the nucleic acid is derived. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones. For example, the isolated nucleic acid is a purified cDNA or RNA polynucleotide. Isolated nucleic acid molecules also include messenger ribonucleic acid (mRNA) molecules and double stranded synthetic polynucleotides such as poly I:C.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

As used herein, the term “about” in the context of a numerical value or range means ±10% of the numerical value or range recited or claimed, unless the context requires a more limited range.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### DESCRIPTION OF THE DRAWINGS

FIG. 1 is a schematic of the immune response role in periodontal disease (PD). The infection of PD typically leads to the formation of activated dendritic cells, which lead to generation of effector T-cells, and chronic inflammation in the tissue that over time results in bone resorption.

FIG. 2 is a schematic of an approach to ameliorate PD inflammation and promote bone regeneration in an embodiment of the present invention. The gel delivered into the site of inflammation first releases GM-CSF and TSLP, to promote formation of tolerant DCs (tDCs) from immature DCs, and block DC activation. The increased ratio of tolerant DCs/activated DCs promotes formation of regulatory T-cells (Tregs), and inhibit effector T-cells. This reduces process inflammation and accompanying bone resorption, and instead promotes resolution of inflammation. The gel releases pDNA encoding for BMP-2 as inflammation subsides, and local BMP-2 expression drives bone regeneration. Bracket A addresses the relation between gel-delivery of GM-CSF and TSLP and subsequent generation of tDCs. Bracket B shows the resultant impact on formation of Tregs and inflammation, and bracket C shows on-demand pDNA delivery from gels and the impact on bone regeneration following amelioration of inflammation.

FIGS. 3A-C are graphs and FIG. 3D is a set of images showing data related to the concentration dependent effects of GM-CSF on DC proliferation, recruitment, activation and emigration in vitro. (3A) shows the in vitro recruitment of JAWSII DCs induced by the indicated concentrations of GM-CSF in transwell systems. Migration counts measured at 12 hours. (3B) is the effects of GM-CSF concentration on the proliferation of JAWSII DCs. 0 (white bar), 50 (grey bar), and 500 ng/ml (black bar) of GM-CSF. (3C) shows the effects of the indicated concentrations of GM-CSF on JAWS II DC emigration from the top wells of transwell systems toward media supplemented with 300 ng/ml CCL19. Migration counts taken at 6 hours. (3D) are representative photomicrographs of TNF-α and LPS stimulated JAWSII DCs cultured in 5-50 or 500 ng/ml GM-CSF and stained for the activation markers MHCII and CCR7. Scale bar in (3D)—20 μm. Values in (3A-3C) represent mean and standard deviation (n=4); * P<0.05; ** P<0.01

FIGS. 4A-F are graphs and images showing data on the in vivo control of DC recruitment and programming. (4A) is the release profile of GM-CSF from polymers that demonstrates a large initial burst, to create high early concentrations of GM-CSF in tissue. (4B) shows H&E staining of tisse sections following explantation from subcutaneous pockets in the backs of C57BL/6J mice after 14 days: Blank polymers, and GM-CSF (3000 ng) loaded polymers. (4C) shows FACS plots of cells isolated from explanted polymers after 28 days and stained for the DC markers, CD11c and CD86 implanted. Numbers in FACS plots indicate the percentage of the cell population positive for both markers. (4D) is the percentage of total cells that were positive for the DC markers CD11c and CD86, in blank (-∘-) and GM-CSF (-•-) loaded polymers as a function of time post implantation. (4E) The total number of DCs isolated from blank (-∘-) and GM-CSF (-•-) loaded polymers as a function of time post implantation. (4F) The fractional increase in CD11c(+)CD86(+) DCs isolated from polymers at day-14 after implantation in response to doses of 1000, 3000 and 7000 ng of GM-CSF as compared to the control. Scale bar—500 μm. Values in 4A, 4D, 4E, and 4F represent mean and standard deviation (n=4 or 5); * P<0.05; ** P<0.01.

FIGS. 5A-C are graphs, FIGS. 5D and E are images, and FIG. 5F is a Table, demonstrating the potency of a material system that delivers TSLP and GM-CSF to PD lesion in induction of tolerogenic DC. FIGS. 5A-5C shows cytokine production by CD11+DC induced in vitro from bone marrow cells with GM-CSF in the presence or absence of TSLP, VIP, or TGF-β (7 day incubation). The in vitro incubation of mononuclear cells isolated from the bone marrow (BM) of C57BL/6 mice with GM-CSF and TSLP (100 ng/ml, respectively) for 7 days up-regulated the differentiation of tolerogenic DC that produced high IL-10 (5A) and low IL-6 (5B) and IL-12 (5C). While TGF-β (100 ng/ml) also showed a similar trend to TSLP in the induction of tolerogenic DC, VIP did not up-regulate the ability of DCs to produce IL-10. The surface phenotypes of CD11c+DC in the BM culture were monitored by flow cytometry and the proportionality of each phenotype is expressed as a percent (%) of the total mononuclear cells (MNC) (FIG. 5, Table 1). The double-color confocal microscopy showed that the gingival injection of gel (1.5 μl) with GM-SCF (1 μg) and TSLP (1 μg) increased CD11c+ cells which produce IL-10 in the mouse periodontal bone loss lesion (5E; 7 days after injection), compared to the control bone loss lesion which did not received injection (5D) Table 1 shows all phenotypes (5F).

FIGS. 6A-B are graphs demonstrating control over local T-cell numbers, and antigen-specific CD8 T-cells. (6A) FACS histograms of CD8(+) cell tissue infiltration with blank vehicle (gray line), vehicle loaded with 3000 ng GM-CSF and 100 μg CpG-ODN alone (dashed line), and vehicle loaded with GM-CSF and antigens (black line). (6B) Characterization of TRP2-specific CD8 T-cells. Splenocytes from naïve mice (naïve) and mice receiving vehicles containing antigen+GM-CSF+ CpG at day 30 (vaccinated) were stained with anti-CD8-FITC Ab, anti-TCR-APC Ab, and Kb/TRP2 pentamers. The ellipitical gates in the upper right quadrant represent the TRP2-specific, CD8(+) T cells and numbers provide percentage of positive cells. Values represent the mean.

FIG. 7 is a set of images showing vertical bone loss induced in a mouse model of PD. 7A is an image of a human clinical case of vertical periodontal bone loss (picture taken at the flap operation). 7B shows GTR-membrane applied onto the vertical bone loss. 7a-7f are anatomical demonstration of vertical bone loss induced in the mouse model of periodontitis. Thirty days following PPAIR-induction in the mice harboring oral Pp by systemic immunization (s.c.) with fixed Aa, animals were sacrificed and defleshed. 7a and 7b: control mice which did not receive immunization with fixed Aa; 7c-7e: mice developed vertical periodontal bone loss around the maxillary molars by systemic immunization with fixed Aa; 7g: histochemical (HE-staining) image of decalcified tissue section of control periodontally healthy mouse; 7h: histochemical (HE-staining) image of mouse which developed PD accompanied by vertical periodontal bone loss (higher magnification image clearly demonstrates extensive neutrophil infiltration).

FIGS. 8A-F are graphs demonstrating that adoptive transfer of ex vivo-expanded Treg to Pp-harboring mice abrogated periodontal bone resorption induced by PPAIR. Following the protocol reported by Zheng et al., these result show ex vivo expansion of FOXP3+CD25+ T cells by culture of spleen cells isolated from Aa-immunized mice (i.p. injection of Aa 10¹⁰/mouse) in the presence of recombinant human TGFb1 (Peprotech), mouse IL-2 (Peprotech), and fixed Aa, as antigens. After ex vivo stimulation for 3 days, the percentage of FOXP3+CD25+ Treg cells in the total lymphocytes increased from 5.5% on day-0 to 15.0% on day-3 (upper 2 figures). Similarly, the percentage of FOXP3+CD4+ Treg cells also increased in the culture (lower 2 figures). After 6 days of ex vivo stimulation, the percentage of FOXP3+CD25+ cells reached 23.3% of the total lymphocytes and 79.8% of the total CD4 T cells. The CD4+ cells were isolated by the magnet beads-based negative selection technique (TGF/IL-2/Aa/CD4+ T cells). TGF/IL-2/Aa/CD4+ Treg cells were labeled with CFSE (5 μM, in PBS, 8 min, MolecularProbe) and adoptively transferred (10⁶/mouse). The localization of CFSE-labeled cells was confirmed by flow cytometry in gingival tissue and cervical lymph nodes (not shown). The TGF/IL-2/Aa/CD4+ Treg cells (2×10⁴/well) were treated with Mitomycin C (MMC) and co-cultured with Aa-specific Th1 effector cells (2×10⁴/well) in the presence of MMC-treated spleen APC (2×105/well) and Aa antigens. CD25+ cells in original spleen CD4+ T cells were depleted by cytotoxic anti-CD25 monoclonal antibody (PC61, rat IgG2a, Pharmingen) in the presence of mouse complement sera (Sigma). Such CD25-depleted spleen CD4+ T cells were also included after adjusting the cell number. Proliferation of Th1 effector cells was monitored by 3H-thymidine assay (4 days), and sRANKL concentration in the culture supernatant was measured by ELISA (8B). The TGF/IL-2/Aa/CD4+ cells were also adoptively transferred into Pp-harboring mice, and bone resorption (8C), concentration of IFN-g (8D), sRANKL (8E) and IL-10 (8F) in the gingival tissue homogenates were all measured on Day-30. *, Significantly different from control by Student's t test (P<0.05). **, Significantly different from the Aa (s.c.) injection alone (*) by Student's t test (P<0.05).

FIGS. 9A-O are graphs and images showing expansion of FOXP3+ T cells in mouse gingival tissue and local lymph nodes (LN) by GM-CSF/TSLP delivery polymer. FOXP3-EGFP-KI mice which previously developed periodontal bone-resorption-socket (maxillary molars) by PPAIR-mediated PD induction received a gingival injection of a total 1.5 μl of (1) control empty polymer, (2) polymer with GM-CSF (1 μg), and (3) polymer with GM-CSF (1 μg)+ TSLP (1 μg). The local cervical lymph nodes (CLN) and maxillary jaws were removed from the sacrificed animals at Day-7 after the injection of polymer. EGFP+ cells (=FOXP3+ Treg cells) in the CLN were monitored by flow cytometry (9A, 9B and 9C). The presence of FOXP3+ Treg cells in the mouse periodontal bone loss lesion was evaluated using a fluorescent confocal microscope (9D-9K). (9D): illustration indicating the anatomical objects (tooth root, alveolar bone and inflammatory connective tissue), (9H): histochemical image (HE-staining) of periodontal bone loss lesion, (9E-9G): bright field images, (9I-9K): fluorescent images. (9E, 9H and 9I): adjacent section of a mouse which did not receive polymer injection, (9F, 9J): a mouse receiving polymer injection with GM-CSF, (9G, 9K): a mouse receiving polymer injection with GM-CSF+ TSLP. Mouse gingival tissue in the bone loss lesion that received GM-CSF/TSLP delivery polymer showed CD11c+ cells and IL-10 around the FOXP3+ T cells infiltrating in the foci (9N, 9O), whereas the control bone loss lesion did not receive polymer injection showed little or no CD11C+ cells or IL-10 in the tissue where the infiltrate of FOXP3 cells was also low (9L, 9M).

FIGS. 10A-D are images demonstrating that polymeric delivery of PEI-condensed pDNA encoding BMP leads to bone regeneration. Implantation of scaffolds led to (10A) long-term (15 week) expression of human BMP-4 in mice (immunohistochemistry; arrows indicate positive cells), and (10B) significant regeneration of bone in critical size cranial defects, as compared to blank polymers. Circles denote original area of bone defect, bone within the circle represents newly regenerated bone tissue. Statistically significant increases in the defect area filled with osteoid (10C) and mineralized tissue (10D), were found with condensed pDNA delivery, as compared to blank polymers, or polymers loaded with an equivalent quantity of non-condensed pDNA. All data at 15 weeks, and values represent mean and standard deviation. The data demonstrate control over the timing of pDNA release from alginate gels via control over gel degradation rate.

FIGS. 11A-B are line graphs demonstrating precise control over the timing of pDNA release from alginate gels with ultrasound. Alginate gels encapsulating pDNA were incubated in tissue culture medium, and an ultrasound transducer was placed in the medium. Irradition (1 W) was applied to gels for 15 min daily; the release rate of pDNA was analyzed by collecting medium and quantifying pDNA in the solution. The base release rate of pDNA was minimal from the high molecular weight, slowly degrading gels used in these studies.

FIG. 12 is a graph showing pDNA release rate.

FIG. 13 is a schematic of an in vitro Treg development assay.

FIG. 14A is a diagram showing an overhead view of a petri dish, light shading represents the collagen and DCs while the darker shading (inner circle) represents the alginate gel).

FIGS. 14B-C are dot plots showing bone marrow-derived dendritic cell chemokinesis in vitro to alginate containing hydrogels with or without GM-CSF. FIG. 14B (no GM-CSF);

FIG. 14C (GM-CSF mixed in with alginate).

FIG. 14D is a list of average migration speed of dendritic cells in the presence of GM-CSF and in the absence of GM-CSF (control).

FIG. 15 is a photograph of alginate gel scaffold material under the skin of a mouse. Scale bar is 5 mm.

FIGS. 16A-B are a series of photomicrographs showing recruitment of DCs to GM-CSF loaded alginate gels in vivo. FIG. 16A shows alginate gels without GM-CSF, and FIG. 16B shows alginate gels containing GM-CSF.

FIG. 16C is a bar graph showing a quantification of cells in blank (alginate without GM-CSF) and GM-CSF loaded alginate gels.

FIG. 17 is a series of photomicrographs showing expression of Forkhead box P3 (FoxP3) in cells adjacent to alginate gels releasing GM-CSF and Thymic stromal lymphopoietin (TSLP) in vivo. Gels containing 3 μg of GM-CSF and 0 μg (A, left panel) or 1 μg (B, right panel) of TSLP were explanted 7 days after injection. White dotted lines indicate the border between the dermal tissue (left) and the alginate gels (right). Scale bars are 50 μm.

FIG. 18 is a line graph showing establishment of a murine type 1 diabetes model.

FIG. 19 is a line graph showing quantification of euglycemic cells following administration of scaffolds containing PLGA-dex, ova, and GM-CSF; PLGA, ova, and GM-CSF, PLGA-dex, BSA and GM-CSF; and PLGA-dex and ova.

FIG. 20 is a bar graph showing ovalbumin-specific IgE in serum following vaccination. The following vaccination groups were tested: no primary vaccination; Ova scaffolds; Ova+GM-CSF scaffolds; Ova+GM-CSF+ CpG scaffolds; and Bolus intraperitoneal (IP) injection of Ova+GM-CSF+ CpG)/no scaffold. These data show that vaccination does not elicit pathogenic IgE antibodies.

FIG. 21 is a bar graph showing splenocyte interferon-γ (IFN-gamma) elaboration following ovalbumin administration.

FIG. 22 is a bar graph showing attenuation of anaphylactic shock following vaccination with scaffolds containing CpG, GM-CSF, and ovalbumin. Temperature of test animals was measured following vaccination and subsequent intraperitoneal challenge with ovalbumin.

FIG. 23A is a flow cytometry histogram showing FACS staining for CD11c in dexamethasone treated BMDC. FIG. 23B is a flow cytometry histogram showing FACS staining for MHC II in dexamethasone treated BMDC. FIG. 23C is a flow cytometry histogram showing FACS staining for CD80 in dexamethasone treated BMDC. FIG. 23D is a flow cytometry histogram showing FACS staining for CD86 in dexamethasone treated BMDC. Representative images of 3 or more trials are displayed.

FIG. 24A is a flow cytometry histogram showing FACS staining for CD11c in dexamethasone and LPS treated BMDC. FIG. 24B is a flow cytometry histogram showing FACS staining for MHC II in dexamethasone and LPS treated BMDC. FIG. 24C is a flow cytometry histogram showing FACS staining for CD80 in dexamethasone and LPS treated BMDC. FIG. 24D is a flow cytometry histogram showing FACS staining for CD86 in dexamethasone and LPS treated BMDC. Representative plots of 3 or more trials are displayed. FIG. 24E is a set of flow cytometry histograms showing the effects of various doses of dexamethasone on FACS staining for MHC II surface expression in a subset of CD11c+ gated cells.

FIG. 25A is a graph showing the effects of dexamethasone treated DCs on T cell proliferation. FIG. 25B is a graph showing the effects of dexamethasone treated DC on DC cell number. Control: cells left untreated. Dex Ct: cells treated with buffer without dexamethasone. ANOVA with post hoc Tukey. n=4 for both experiments. * p=0.04, **p=0.01, ***p=0.29 (A). * reflects the comparison of control to dexamethasone 10⁻⁶ M treated groups (B), p<0.05.

FIGS. 26A-C depict transwell migration of Jaws II DC toward dexamethasone. FIG. 26B shows migration of Jaws II cells cultured in the presence of dexamethasone toward CCL19. FIG. 26C shows migration of Jaws II cells cultured in the presence of dexamethasone toward CCL 20. Samples were normalized to the average number of cells that migrated per experiment. n=3-8, * p=0.017, ** p=0.006, *** p=0.05, using ANOVA with Tukey. FIGS. 26A-C show the number of dendritic cells recruited to various cytokines/chemokines depending on dexamethasone concentration.

FIG. 27A is an illustration of dexamethasone coupled to a succinic anhydride via primary alcohol (*) and subsequently to a peptide through the carboxylic acid of the hemisuccinate (**). FIG. 27B is a schematic showing a solid phase synthesis coupling strategy incorporating the dexamethasone hemisuccinate derivative, 4-pregnadien-9α-fluoro-16α-methyl-11β, 17, 21-triol-3, 20-dione 21-hemisuccinate, to the N-terminus of a growing peptide prior to cleavage and side chain deprotection. FIG. 27C is a LC-MS spectrum depicting the purity of the final product after RP-HPLC purification on a C18 column. FIG. 27D is a mass spectrum depicting the purity of the final product after the RP-HPLC purification. FIGS. 27A-D depict a method for dexamethasone-immunoconjugate design and synthesis.

FIG. 28A is a set of flow cytometry histograms showing the surface expression of MHC II. FIG. 28B is a set of flow cytometry histograms showing the surface expression of the co-stimulatory molecule, CD80. FIG. 28C is a set of flow cytometry histograms showing the surface expression of the co-stimulatory molecule, CD86. FIG. 28D is a bar graph showing the elaboration of IL-12p70 in the various treatments. FIG. 28E is a set of flow cytometry histograms showing staining for SIINFEKL bound to H2Kb in BMDC pulsed for 2 hours with 0 μM SIINFEKL, 3 μM SIINFEKL, 3 μM SIINFEKL plus 3 μM dexamethasone-SIINFEKL, or 3 μM dex-SIINFEKL alone. The samples from left to right (lightest to darkest) are isotype control, 0 μM SIINFEKL, 3 μM dexamethasone-SIINFEKL, 3 NM SIINFEKL, and 3 μM SIINFEKL and 3 μM dexamethasone-SIINFEKL. For all histograms representative plots from two experiments with multiple samples are shown. In the IL-12p70 plot, p is less than 0.014 for all comparisons except for untreated cells vs dexamethasone/LPS treated samples and dexamethasone/LPS vs dexamethasone-SIINFEKL/LPS treated cells which are not statistically different. Analysis by ANOVA followed by Tukey, n=3-6. FIGS. 28A-E show the effects of dexamethasone-SIINFEKL on DC maturation and antigen presentation.

FIG. 29 is a panel of images of B3Z cells showing the level of dexamethasone-SIINFEKL MHC Class I presentation to T cells in an X-gal assay. Scale bar equals 50 m.

FIG. 30A depicts the relationship between β-galactosidase activity and SIINFEKL or dexamethasone-SIINFEKL in pulsed DC. Statistical analysis was completed by comparing SIINFEKL groups to the dexamethasone-SIINFEKL groups with equivalent peptide concentrations; the bars represent p<0.05 for SIINFEKL versus the dexamethasone-SIINFEKL groups, ANOVA and Bonferroni. FIG. 30B is a magnification of the dexamethasone-SIINFEKL group in FIG. 30A. All groups were compared against each other, and p was less than 0.05 for the comparison between No peptide and D-SIINFEKL 100 nM and No peptide and D-SIINFEKL 1000 nM, ANOVA and Tukey. n=4. FIGS. 30A-B show the level of dexamethasone-SIINFEKL MHC Class I presentation to T cells in a CPRG assay.

FIG. 31 is a set of flow cytometry histograms showing the effects of a dexamethasone conjugate on proliferation of OT-I T cells. In rows A-C, BMDC were pretreated with no antigen (B) or dexamethasone-SIINFEKL (C). Row A depicts the control condition whereby T cells were left in culture without BMDC. In rows D-G, BMDC were treated with ovalbumin and either media alone (D), dexamethasone (E), dexamethasone bound to an irrelevant peptide (F), or dexamethasone-SIINFEKL (G). T cells were gated on FSC and SSC to capture the live lymphocytes. The samples are normalized to the peak height and represent a typical plot of three samples.

FIG. 32A is a plot of the clinical score with time in days. FIG. 32B lists disease metrics. FIG. 32C is a plot of results of a trial. FIG. 32D is a table of results from the trial. FIGS. 32A-B show results of a prophylactic trial in C57BL/6 mice left untreated (Untreated control), mice treated s.c. with MOG (200 μg) and dexamethasone (30 μg) in IFA (D+MOG), or mice treated with dexamethasone conjugated to MOG (240 μg, equimole to the MOG and dexamethasone applied alone) in IFA (D-MOG). Seven days later disease was induced (day 0) and the animals were monitored for 1 month (A). FIGS. 32C-D show results of a prophylactic trial in which mice were left untreated or treated s.c. with D-MOG (100 μg), D-MOG+GM-CSF (3 μg), or GM-CSF (3 μg) and D-MOG (100 μg) with PLG scaffolds. Four days later, disease was induced. The error bars in FIGS. 32A and 32C represent the SEM. α: p<0.001, D-MOG to untreated; β: p<0.05 (one-way), D-MOG to untreated; γ: p<0.01, D+MOG to untreated; τ: p<0.01, D+MOG to D-MOG, λ: p<0.05, D+MOG to untreated; δ: p<0.01, D-MOG to untreated; ζ: p<0.05, D-MOG to untreated all using ANOVA/Bonferroni comparisons between groups or chi-square test. ε: p=0.044 comparing D-MOG to D+MOG using a one-way student's t-test. FIGS. 32A-D show that prophylactic treatment with dexamethasone conjugated to MOG₃₅₋₅₅ delays the onset and attenuates disease severity in mice with EAE.

FIG. 33A is a graph showing the rate of release of dexamethasone from PLG materials used in the EAE trial described in Example 6. FIG. 33B is a graph showing the rate of release of dexamethasone from PLG scaffolds with immunoconjugate loaded into the microparticles during the WOW emulsion step (DMOG Encapsulated in Microspheres), macroporous cryogels with the immunoconjugate chemisorbed to the microparticles prior to gas-foaming (DMOG Chemisorbed), or macroporous cryogels with the immunoconjugate added to the polymerization cocktail (DMOG Encapsulated). n=4-5 samples. The black line (filled in circles) refers to the material used in the EAE trial and in FIG. 33A. FIGS. 33A-B show the rate of release of dexamethasone from various polymeric materials.

FIG. 34A are a set of LC-MS spectra taken at various time points after incubation of Dex-MOG at 37° C. Total ionic current is shown. FIG. 34B is a mass spectrum of peak a (immunoconjugate). FIG. 34C is a mass spectrum of peak b (peptide fragment). FIG. 34D is a mass spectrum of peak c (dexamethasone). FIG. 34E is a graph showing the quantitation of dexamethasone formation and immunoconjugate scission at various time points. FIGS. 34A-E show the scission of Dex-MOG at 37° C.

FIG. 35 is a graph showing the level of dexamethasone-MOG degradation in PLG scaffolds after heat treatment after various time points. The control sample had control immunoconjugate not incorporated into the scaffold. ANOVA with Tukey, p<0.05 for all comparisons to the control sample.

FIG. 36A is a graph showing the effects on antigen specific elaboration of IL-17. FIG. 36B is a graph showing the severity of EAE disease in adoptive transfer mice. FIG. 36C is a table showing the quantification of FIG. 36B. ANOVA with Tukey, n=3-5 animals. Blue bars, θ, p<0.05. FIGS. 36A-C show the ability of the Dex-MOG immunoconjugate to inhibit antigen specific Th17 T cells and to delay disease onset in an adoptive transfer EAE model.

FIG. 37 is a diagram showing antigen conjugation to a model antigen, e.g., a tumor antigen.

FIG. 38 is a series of photographs showing antigen+adjuvant conjugates.

FIG. 39 is a bar graph showing dendritic cell responses to CpG-antigen conjugates.

FIG. 40 is a line graph and a bar graph showing T cell responses to CpG-antigen conjugates.

FIG. 41 is a bar graph showing enhanced CD8 T cell homing to scaffold/vehicles containing conjugates vs. unconjugated antigen.

FIG. 42 is a line graph showing tumor protection.

FIG. 43 is a series of line graphs showing inhibition of tumor growth.

FIG. 44 is a diagram of photo-linkage of antigen to adjuvant.

FIG. 45 is a photograph of an electrophoretic gel showing conjugation of antigen to adjuvant.

FIGS. 46A and B are cartoons comparing (i) the use of an immunoconjugate comprising an antigen and immunomodulatory agent with (ii) the use of an unconjugated antigen and unconjugated immunomodulatory agent (antigen and immunomodulatory agent are not linked but rather exist separately from one another, i.e., not conjugated or covalently bound, in a solution or in/on a scaffold device). FIG. 46A shows an antigen and an immunomodulatory agent contacting different cells, resulting in off target effects. FIG. 46B is a cartoon showing an immunoconjugate that associates with a single cell. The covalent conjugation of the antigen to the immunomodulatory agent results in a single cell being contacted with both compounds. Thus, the components of the immunoconjugate act on a single cell together to have a combination effect, rather than on multiple cells which may result in aberrant effects (such as toxicity or an unwanted immune reaction) or reduced efficacy.

FIG. 47 is a pair of line graphs showing data from three (3) mesoporous silica (MPS) vaccine formulations that were tested: 1) MPS vaccine containing the Gonadotropin-releasing hormone peptide (GnRH) peptide (100 μg), CpG (100 μg) and GM-CSF (1 μg) (unconjugated GnRH), 2) MPS vaccine containing GnRH peptide conjugated to CpG (100 μg of each) and GM-CSF (1 μg) (GnRH-CpG), and 3) MPS vaccine containing the GnRH peptide conjugated to OVA (100 μg peptide), CpG (100 ug) and GM-CSF (1 ug) (GnRH-OVA). Mice were immunized on day 0 and blood serum was collected and monitored subsequently. Antibody against GnRH was measured using an indirect enzyme-linked immunosorbent assay (ELISA), and titer is defined as the highest serum dilution at which the OD value reaches 0.2. Only the MPS vaccine with GnRH conjugated to OVA raised high and long lasting antibody against GnRH.

FIG. 48 is a line graph showing the evaluation of the release kinetics of the GnRH-OVA conjugate from the MPS scaffold. The conjugate was loaded into the MPS scaffold for 8 hours at room temperature (RT). The conjugate was shown release in a sustained manner followed by a burst release.

FIGS. 49A and B are line graphs comparing a MPS vaccine containing the GnRH-OVA conjugate to a bolus vaccine formulation. The MPS vaccine contains 5 mg of MPS loaded with 100 μg of GnRH peptide conjugated to OVA, 100 μg of CpG and 1 μg of GM-CSF. The bolus formulation contains 100 μg of GnRH peptide conjugated to OVA, 100 μg of CpG and 1 μg of GM-CSF. Mice were immunized on day 0 and blood serum was collected and monitored subsequently. The MPS vaccine significantly enhanced IgG1 (A) and IgG2a (B) antibody response against GnRH compared to the bolus formulation.

FIG. 50 is a pair of line graphs showing antibody titers resulting when GnRH peptide was conjugated to Keyhole limpet hemocyanin (KLH) (exemplary model) as the carrier protein. KLH is one of the most widely used and immunogenic carrier proteins used for immunization and antibody production against peptide antigens. MPS vaccine containing the GnRH-KLH conjugate was compared to a bolus vaccine formulation. The MPS vaccine contains 5 mg of MPS loaded with 30 μg of GnRH peptide conjugated to KLH, 100 μg of CpG and 1 μg of GM-CSF. The bolus formulation contains 30 μg of GnRH peptide conjugated to KLH, 100 μg of CpG and 1 μg of GM-CSF. Mice were immunized on day 0 and blood serum was collected and monitored subsequently. The MPS vaccine significantly enhanced IgG1 antibody response against GnRH compared to the bolus formulation.

FIGS. 51A and B are line graphs comparing multiple adjuvants. Three adjuvants were explored in the MPS GnRH-OVA vaccine: CpG, PolyIC and MPLA. Mice were vaccinated with MPS vaccines containing 100 μg GnRH-OVA, 1 μg GM-CSF and 100 μg of CpG, PolyIC or MPLA. All vaccine formulations induced comparable levels of IgG1 antibody against GnRH. Vaccines using CpG induced the highest level of IgG2a antibody against GnRH compared to vaccines using PolyIC or MPLA.

FIG. 52 is a graph comparing different epitopes conjugated to an MPS scaffold. The ovalbumin CD8 epitope (CSIINFEKL) (SEQ ID NO: 18) and ovalbumin CD4 epitope (CISQAVHAAHAEINEAGR) (SEQ ID NO: 19) were conjugated to the MPS scaffold through stable maleimide (sulfhydryl-sulfhydryl)(SMCC) and reducible maleimide (sulfhydryl-sulfhydryl)(SPDP) linkers. Primary amines were first introduced to MPS particles using (3-aminopropyl)triethoxysilane (APTES) and reacted with SMCC and SPDP linkers for 2 hours at room temperature. Cysteine containing peptides were then reacted with SMCC or SPDP modified MPS at 1.2 molar ratio overnight. After the reaction, MPS particles were washed extensively and conjugation efficiency was determined. Through simple adsorption, approximately 40% of the peptides were loaded onto the MPS. However, 100% and 80% conjugation efficiency was achieved through SMCC and SPDP modification, respectively.

FIG. 53 is a pair of graphs evaluating the antigen presentation of CSIINFEKL (SEQ ID NO: 18)-MPS conjugate. 100 nM and 10 nM of CSIINFEKL (SEQ ID NO: 18), SMCC CSIINFEKL (SEQ ID NO: 18)-MPS, SPDP CIINFEKL (SEQ ID NO: 18)-MPS and CIINFEKL (SEQ ID NO: 18) adsorbed to MPS was cultured with bone marrow derived dendritic cells (BMDCs) for 18 hours. Percentage of BMDCs presenting the peptide was quantified using flow cytometry. At 100 nM, SPDP CSIINFEKL (SEQ ID NO: 18)-MPS was presented at comparable levels to CSIINFEKL (SEQ ID NO: 18) adsorbed to MPS. However, at 10 nM, SPDP CIINFEKL (SEQ ID NO: 18)-MPS was significantly better presented compared to CIINFEKL (SEQ ID NO: 18) adsorbed to MPS.

FIG. 54A-C are a set of graphs showing the effect of peptide-MPS conjugates on CD4 T cell proliferation, as evaluated in vitro. BMDCs were stimulated with CISQAVHAAHAEINEAGR (peptide) (SEQ ID NO: 19), CISQAVHAAHAEINEAGR (SEQ ID NO: 19) conjugated to MPS through SMCC (SMCC), and CISQAVHAAHAEINEAGR (SEQ ID NO: 19) conjugated to MPS through SPDP (SPDP) for 18 hours. BMDCs were then washed thoroughly and co-cultured with CD4⁺ T cells from OT-II mice. OT-II mice are enriched for CD4⁺ T cells recognizing the ISQAVHAAHAEINEAGR (SEQ ID NO: 20) peptide. Both SMCC and SPDP peptide-MPS induced significantly higher T cell proliferation compared to peptide stimulation only.

FIG. 55A is a set of images and FIG. 55B is a line graph showing the kinetics of antigen presentation, as evaluated in vivo. Rhodamine labeled CSIINFEKL (CSIINFEKL-Rho) was imaged using IVIS after immunization. Mice were immunized with CIINFEKL-Rho (bolus), CSIINFEKL-Rho conjugated to MPS through SMCC and SPDP linkers (SMCC, SPDP, respectively) and CSIINFEKL-Rho adsorbed onto MPS (ADS). SPDP conjugation of peptide to MPS resulted in prolonged local antigen presence compared to bolus and adsorbed formulations.

### DETAILED DESCRIPTION

Aspects of the present subject matter relate to the surprising discovery that immunoconjugates comprising an antigen covalently linked to an immunomodulatory agent (e.g., a tolerogen or an adjuvant) have enhanced potency and/or activity, e.g., at least about 5, 10, 15, 20, 25, 30, 35, 40, 45, 50, 55, 60, 65, 70, 80, 90, 95, or 100% or 2-fold, 5-fold, or 10-fold increased potency and/or activity. For example, an immunoconjugate comprising an antigen and a tolerogen has enhanced potency or activity in reducing an undesirable immune response (such as an allergic reaction or an autoimmune disease) compared to the unconjugated combination of the antigen and the tolerogen. Likewise, an immunoconjugate comprising an antigen and an immunostimulatory adjuvant (e.g. a TLR ligand or agonist) has enhanced potency and/or activity in increasing an immune response, such as an anti-cancer immune response (e.g., anti-cancer vaccination). Thus, surprisingly, greater efficacy may be achieved with the same amount of antigen and immunomodulatory agent by covalently linking these compounds together.

A non-limiting advantage of this technology is the delivery of both the antigen and the immunomodulatory agent to a particular target (e.g., an immune cell and/or a receptor thereof) at the same time and location. The co-delivery of antigen and immunomodulatory agent as an immunoconjugate not only increases potency and/or activity, but also enhances treatment specificity. Thus, compounds of the present subject matter have increased efficacy with reduced off-target effects.

In various aspects, the dose of the immunomodulatory agent and/or the antigen is less than would otherwise be required if the immunomodulatory agent and/or the antigen was administered singly or without being covalently linked (i.e., conjugated) to the other. Certain implementations of the present subject matter relate to the continuous release of an immunoconjugate in an amount that is less than the amount that would be needed to achieve the desired effect if the antigen and immunomodulatory agent were released in an unconjugated form. The continuous release may be, e.g., from a scaffold device that contains and delivers over time the immunoconjugate locally or systemically. Thus, not only may lower amounts of antigen and immunomodulatory agent be used in immunoconjugate form, but a particularly low amount of the immunoconjugate may be released locally [e.g., subcutaneously, within or near (e.g. proximal to or touching) a tumor, within an oral cavity, or near the site of abberant inflammation] over time. An advantage of this discovery is that immunomodulatory agents that might not be clinically suitable (e.g., due to undesirable side effects) when administered in unconjugated form may be useful in embodiments disclosed herein. Thus, the present subject matter broadens the array of therapeutic agents that may be used to treat subjects afflicted with, e.g., cancer, autoimmune diseases, allergies, asthma, and transplantation graft rejection. Moreover, the increased potency and specificity of immunoconjugates renders them more suitable for preventative and prophylactic treatment than unconjugated antigens and immunomodulatory agents.

The immunoconjugates (antigen+tolerogen), delivery device scaffolds, and systems described herein mediate spatiotemporal presentation of cues that locally control DC activation and bias the immune response towards a non-pathogenic state. The compositions are used to treat subjects that have been identified as suffering from or at risk of developing diseases or disorders characterized by inappropriate immune activation. The biomaterial systems (loaded scaffolds) recruit DCs and promote their activation towards a tolerogenic or non-inflammatory phenotype (autoimmunity/inflammation) or an activated state (allergy/asthma) that corrects an aberrant or misregulated immune response that occurs in a pathologic condition.

For autoimmune disease, the delivery vehicle scaffolds comprise an antigen (autoantigen), a recruitment composition, and a tolerogen. For allergy or asthma, the scaffolds comprise and antigen (allergen), a recruitment composition, and an adjuvant (e.g, a Th1 promoting adjuvant such as CpG). Generation of Treg cells leads to clinical benefit by directing the immune response away from pathogenic T effectors and toward other immune effectors such as Treg, Th1, Th17 arms of the immune system.

The vaccines attenuate diseases of pathogenic immunity by re-directing the immune system from a Th1/Th17 to T regulatory biased immune response (autoimmunity) and a Th2 response to a Th1 biased immune response (allergy/asthma).

### Delivery Scaffolds

Exemplary delivery scaffolds (delivery vehicle structures) were produced using PLG (for allergy or asthma) or alginate (for autoimmune diseases such as diabetes of for periodontitis). PLG was compressed, gas foamed, and leached (porogens (that were later leached) 250 μm to 400 μm made up 90% of the compressed powder) to create a porous material. Gels are typically 1-20% polymer, e.g., 1-5% or 1-2% alginate. Methods of making scaffolds are known in the art and are described in, e.g., U.S. Pat. No. 8,067,237 and PCT International Patent Application Publication No. WO 2009/102465, the entire contents of each of which are incorporated herein by reference. The polymers are preferably crosslinked. For example, 1-2% alginate was crosslinked ionically in the presence of a divalent cation (e.g., calcium). Alternatively, to modify the spatiotemporal presentation of molecules and control degradation, the alginate is crosslinked covalently by derivatizing the alginate chains with molecules by oxidation with sodium periodate and crosslinking with adipic dihydrazide.

Scaffolds and delivery devices comprising scaffolds described herein are small enough to be injected or surgically implanted in to subjects. In some examples, the device is between 0.01 mm³ and 100 mm³, between 1 mm³ and 75 mm³, between 5 mm³ and 50 mm³, between 10 mm³ and 25 mm³, between 1 mm³ and 10 mm³ in size, or less than about 5, 10, 15, 20, 30, 40, 50, 100, 150, 200, or 250 mm³. In some situations, a device comprises the shape of a disc, cylinder, square, rectangle, or string.

### Click Chemistry Linkage of Antigen to Immunomodulatory Agent

A bioorthogonal functional group and the target recognition molecule comprises a complementary functional group, where the bioorthogonal functional group is capable of chemically reacting with the complementary functional group to form a covalent bond. Exemplary bioorthogonal functional group/complementary functional group pairs include azide with phosphine; azide with cyclooctyne; nitrone with cyclooctyne; nitrile oxide with norbornene; oxanorbornadiene with azide; trans-cyclooctene with s-tetrazine; quadricyclane with bis(dithiobenzil)nickel(II).

For example, the bioorthogonal functional group is capable of reacting by click chemistry with the complementary functional group. In some cases, the bioorthogonal functional group comprises transcyclooctene (TOC) or norbornene (NOR), and the complementary functional group comprises a tetrazine (Tz). In some examples, the bioorthogonal functional group comprises dibenzocyclooctyne (DBCO), and the complementary functional group comprises an azide (Az). In other examples, the bioorthogonal functional group comprises a Tz, and the complementary functional group comprises transcyclooctene (TOC) or norbornene (NOR). Alternatively or in addition, the bioorthogonal functional group comprises an Az, and the complementary functional group comprises dibenzocyclooctyne (DBCO).

For example, the target comprises a bioorthogonal functional group and the target recognition molecule comprises a complementary functional group, where the bioorthogonal functional group is capable of chemically reacting with the complementary functional group to form a covalent bond, e.g., using a reaction type described in the table below, e.g., via click chemistry.

By bioorthogonal is meant a functional group or chemical reaction that can occur inside a living cell, tissue, or organism without interfering with native biological or biochemical processes. A bioorthogonal functional group or reaction is not toxic to cells. For example, a bioorthogonal reaction must function in biological conditions, e.g., biological pH, aqueous environments, and temperatures within living organisms or cells. For example, a bioorthogonal reaction must occur rapidly to ensure that covalent ligation between two functional groups occurs before metabolism and/or elimination of one or more of the functional groups from the organism. In other examples, the covalent bond formed between the two functional groups must be inert to biological reactions in living cells, tissues, and organisms.

Exemplary bioorthogonal functional group/complementary functional group pairs are shown in the table below.

| Functional | Paired | | Reaction type |
|---|---|---|---|
| group | with | Functional group | (Reference) |
| Azide | | phosphine | Staudinger ligation (Saxon et al. Science 287(2000): 2007-10) |
| Azide | | Cyclooctyne, e.g., dibenzocyclooctyne, or one of the cyclooctynes shown below: 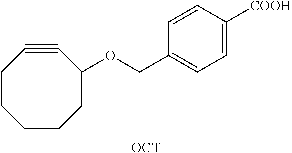 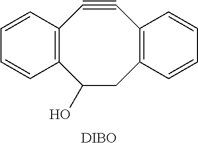 | Copper-free click chemistry (Jewett et al. J. Am. Chem. Soc. 132.11(2010): 3688-90; Sletten et al. Organic Letters 10.14(2008): 3097-9; Lutz. 47.12(2008): 2182) |
| | | 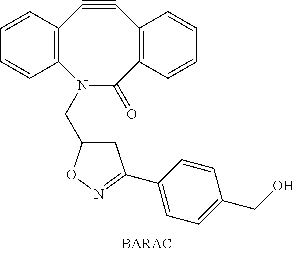 [SMILES: O=C1c2ccccc2C#Cc2ccccc2N1CC1CC(c2ccc(CO)cc2)=NO1] | |
| | | 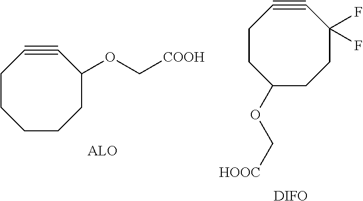 | |
| | | 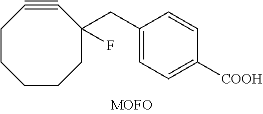 [SMILES: O=C(O)c1ccc(CC2(F)C#CCCCCC2)cc1] | |
| | | 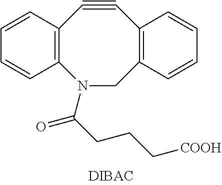 [SMILES: O=C(O)CCCC(=O)N1Cc2ccccc2C#Cc2ccccc21] | |
| | | 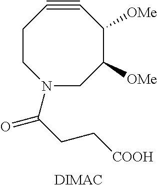 [SMILES: CO[C@H]1C#CCCN(C(=O)CCC(=O)O)C[C@@H]1OC] | |
| Nitrone | | cyclooctyne | Nitrone Dipole Cycloaddition (Ning et al. 49.17(2010): 3065) |
| Nitrile oxide | | norbornene | Norbornene |
| | | | Cycloaddition |
| | | | (Gutsmiedl et al. |
| | | | Organic Letters |
| | | | 11.11(2009): 2405-8) |
| Oxanorbornadine | | azide | Oxanorbornadiene |
| | | | Cycloaddition (Van |
| | | | Berkel et al. |
| | | | 8.13(2007): 1504-8) |
| Transcyclooctene | | s-tetrazine | Tetrazine ligation |
| | | | (Hansell et al. J. Am. |
| | | | Chem. Soc. |
| | | | 133.35(2011): 13828-31) |
| Nitrile | | 1,2,4,5-tetrazine | [4 + 1] cycloaddition |
| | | | (Slackman et al. Organic |
| | | | and Biomol. Chem. |
| | | | 9.21(2011): 7303) |
| quadricyclane | | Bis(dithiobenzil)nickel(II) | Quadricyclane Ligation |
| | | | (Sletten et al. J. Am. |
| | | | Chem. Soc. |
| | | | 133.44(2011): 17570-3) |
| Ketone or | | Hydrazines, hydrazones, oximes, amines, ureas, thioureas, | Non-aldol carbonyl |
| aldehyde | | etc. | chemistry |
| | | | (Khomyakova E A, et al. |
| | | | Nucleosides Nucleotides |
| | | | Nucleic Acids. 30(7-8) |
| | | | (2011) 577-84 |
| Thiol | | maleimide | Michael addition |
| | | | (Zhou et al. 2007 |
| | | | 18(2): 323-32.) |
| Dienes | | dieoniphiles | Diels Alder (Rossin et |
| | | | al. Nucl Med. (2013) |
| | | | 54(11): 1989-95) |
| Tetrazene | | norbornene | Norbornene click |
| | | | chemistry (Knight et al. |
| | | | Org Biomol Chem. 2013 |
| | | | Jun 21; 11(23): 3817-25.) |

In some examples, a target molecule comprises a bioorthogonal functional group such as a trans-cyclooctene (TCO), dibenzycyclooctyne (DBCO), norbornene, tetrazine (Tz), or azide (Az). In other example, a target recognition molecule (e.g., on the device) comprises a bioorthogonal functional group such as a trans-cyclooctene (TCO), dibenzycyclooctyne (DBCO), norbornene, tetrazine (Tz), or azide (Az). TCO reacts specifically in a click chemistry reaction with a tetrazine (Tz) moiety. DBCO reacts specifically in a click chemistry reaction with an azide (Az) moiety. Norbornene reacts specifically in a click chemistry reaction with a tetrazine (Tz) moiety. For example, TCO is paired with a tetrazine moiety as target/target recognition molecules. For example, DBCO is paired with an azide moiety as target/target recognition molecules. For example, norbornene is paired with a tetrazine moiety as target/target recognition molecules.

The exemplary click chemistry reactions have high specificity, efficient kinetics, and occur in vivo under physiological conditions. See, e.g., Baskin et al. Proc. Natl. Acad. Sci. USA 104(2007):16793; Oneto et al. Acta biomaterilia (2014); Neves et al. Bioconjugate chemistry 24(2013):934; Koo et al. Angewandte Chemie 51(2012):11836; and Rossin et al. Angewandte Chemie 49(2010):3375.

As described above, click chemistry reactions are particularly effective for labeling biomolecules. They also proceed in biological conditions with high yield. Exemplary click chemistry reactions are (a) Azide-Alkyne Cycloaddition, (b) Copper-Free Azide Alkyne Cycloaddition, and (c) Staudinger Ligation shown in the schemes below.

### A) Azide-Alkyne Cycloaddition

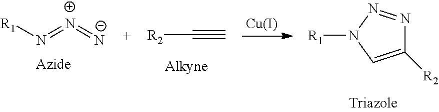

### B) Copper-Free Azide-Alkyne Cycloaddition

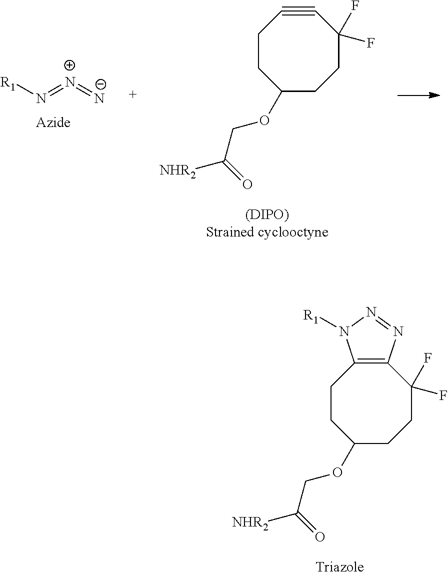

### C) Staudinger Ligation

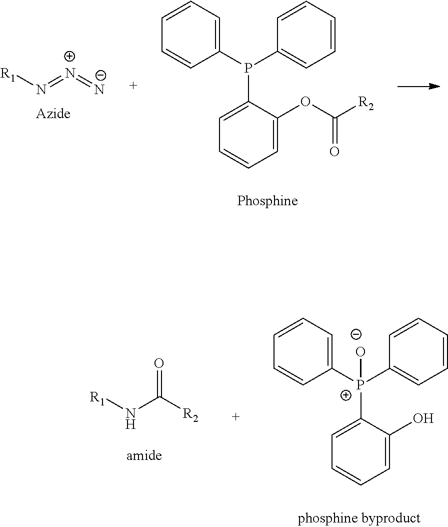

Methods of making delivery scaffolds or devices using two or more different polymers may also involve click chemistry. The invention provides a hydrogel comprising a first polymer and a second polymer, where the first polymer is connected to the second polymer by formula (I):

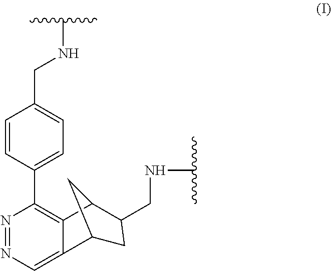 [SMILES: *NCc1ccc(-c2nncc3c2C2CC3CC2CN*)cc1]

or by formula (II):

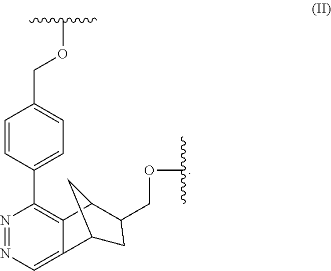 [SMILES: *OCc1ccc(-c2nncc3c2C2CC3CC2CO*)cc1]

In some embodiments, the hydrogel comprises a plurality of formula (I) or formula (II). The hydrogel may comprise an interconnected network of a plurality of polymers, e.g., including a first polymer and a second polymer. For example, the polymers are connected via a plurality of formula I or formula II. For example, the first polymer and/or the second polymer comprise the same type of polymer. In some examples, the first polymer and/or the second polymer comprise a polysaccharide. For example, the first polymer and the second polymer both comprise a polysaccharide. In some embodiments, the first polymer and/or the second polymer comprise alginate, polyethylene glycol (PEG), gelatin, hyaluronic acid, collagen, agarose, or polyacrylamide. In a preferred embodiment, the first polymer and the second polymer comprise alginate. Such click crosslinked hydrogels are described in PCT International Patent Application Publication No. WO/2015/154078, published Oct. 8, 2015; and U.S. Ser. No. 61/975,375; the contents of each of which is hereby incorporated by reference in their entireties.

Immunoconjugates for Eliciting and/or Augmenting an Immune Response

Antigens are conjugated to adjuvants or immunopotentiating agents, e.g., TLR ligands or agonists and administered to subjects to activate immunity or increase the level of an immune response to the antigen delivered. Exemplary TLR ligands and the cells on which the TLR receptors are expressed are shown in the table below.

| Receptor | Ligand(s) | Cell types |
|---|---|---|
| TLR 1 | multiple triacyl lipopeptides | monocytes/macrophages |
| | | a subset of dendritic cells |
| | | B lymphocytes |
| TLR 2 | multiple glycolipids | monocytes/macrophages |
| | multiple lipopeptides | neutrophils |
| | multiple lipoproteins | Myeloid dendritic cells |
| | lipoteichoic acid | Mast cells |
| | HSP70 | |
| | zymosan (Beta-glucan) | |
| | Numerous | others |
| TLR 3 | double-stranded RNA poly | Dendritic cells |
| | I:C | B lymphocytes |
| TLR 4 | lipopolysaccharide | monocytes/macrophages |
| | several heat shock proteins | neutrophils |
| | fibrinogen | Myeloid dendritic cells |
| | heparan sulfate fragments | Mast cells |
| | hyaluronic acid fragments | B lymphocytes |
| | nickel | Intestinal epithelium |
| | Various opioid drugs | |
| TLR 5 | Bacterial flagellin | monocyte/macrophages |
| | profilin | a subset of dendritic cells |
| | | Intestinal epithelium |
| TLR 6 | multiple diacyl lipopeptides | monocytes/macrophages |
| | | Mast cells |
| | | B lymphocytes |
| TLR 7 | imidazoquinoline | monocytes/macrophages |
| | loxoribine (a guanosine | Plasmacytoid dendritic cells |
| | analogue) | B lymphocytes |
| | bropirimine | |
| | single-stranded RNA | |
| TLR 8 | small synthetic compounds; | monocytes/macrophages |
| | single-stranded RNA | a subset of dendritic cells |
| | | Mast cells |
| TLR 9 | unmethylated CpG | monocytes/macrophages |
| | Oligodeoxynucleotide DNA | Plasmacytoid dendritic cells |
| | | B lymphocytes |
| TLR 10 | unknown | |
| TLR 11 | Profilin | monocytes/macrophages |
| | | liver cells |
| | | kidney |
| | | urinary bladder epithelium |
| TLR 12 | Profilin | Neurons |
| | | plasmacytoid dendritic cells |
| | | conventional dendritic cells |
| | | macrophages |
| TLR 13 | bacterial ribosomal RNA | monocytes/macrophages |
| | sequence ″CGGAAAGACC″ | conventional dendritic cells |
| | (SEQ ID NO: 16) | |

Any adjuvant is suitable for covalent linkage to an antigen, e.g., a purified tumor antigen or mixture of tumor antigens such as a tumor cell lysate preparation. Exemplary adjuvants include TLR ligands such as those described as follows: TLR-1:—Bacterial lipoprotein and peptidoglycans; TLR-2:—Bacterial peptidoglycans; TLR-3:—Double stranded RNA;

TLR-4:—Lipopolysaccharides; TLR-5:—Bacterial flagella; TLR-6:—Bacterial lipoprotein; TLR-7:—Single stranded RNA; TLR-8:—Single stranded RNA; TLR-9:—CpG DNA; TLR-10:—TLR-10 ligand.

### Cytosine-Guanosine (CpG) Oligonucleotide (CpG-ODN) Sequences

CpG sites are regions of deoxyribonucleic acid (DNA) where a cysteine nucleotide occurs next to a guanine nucleotide in the linear sequence of bases along its length (the “p” represents the phosphate linkage between them and distinguishes them from a cytosine-guanine complementary base pairing). CpG sites play a pivotal role in DNA methylation, which is one of several endogenous mechanisms cells use to silence gene expression. Methylation of CpG sites within promoter elements can lead to gene silencing. In the case of cancer, it is known that tumor suppressor genes are often silenced while oncogenes, or cancer-inducing genes, are expressed. CpG sites in the promoter regions of tumor suppressor genes (which prevent cancer formation) have been shown to be methylated while CpG sites in the promoter regions of oncogenes are hypomethylated or unmethylated in certain cancers. The TLR-9 receptor binds unmethylated CpG sites in DNA.

Various compositions described herein comprise CpG oligonucleotides. CpG oligonucleotides are isolated from endogenous sources or synthesized in vivo or in vitro. Exemplary sources of endogenous CpG oligonucleotides include, but are not limited to, microorganisms, bacteria, fungi, protozoa, viruses, molds, or parasites. Alternatively, endogenous CpG oligonucleotides are isolated from mammalian benign or malignant neoplastic tumors. Synthetic CpG oligonucleotides are synthesized in vivo following transfection or transformation of template DNA into a host organism. Alternatively, Synthetic CpG oligonucleotides are synthesized in vitro by polymerase chain reaction (PCR) or other art-recognized methods (Sambrook, J., Fritsch, E. F., and Maniatis, T., Molecular Cloning: A Laboratory Manual. Cold Spring Harbor Laboratory Press, NY, Vol. 1, 2, 3 (1989), herein incorporated by reference).

CpG oligonucleotides are presented for cellular uptake by dendritic cells. For example, naked CpG oligonucleotides are used. The term “naked” is used to describe an isolated endogenous or synthetic polynucleotide (or oligonucleotide) that is free of additional substituents. In another embodiment, CpG oligonucleotides are bound to one or more compounds to increase the efficiency of cellular uptake. Alternatively, or in addition, CpG oligonucleotides are bound to one or more compounds to increase the stability of the oligonucleotide within the scaffold and/or dendritic cell. CpG oligonucleotides are optionally condensed prior to cellular uptake. For example, CpG oligonucleotides are condensed using polyethylimine (PEI), a cationic polymer that increases the efficiency of cellular uptake into dendritic cells to yield cationic nanoparticles. CpG oligonucleotides may also be condensed using other polycationic reagents to yield cationic nanoparticles. Additional non-limiting examples of polycationic reagents that may be used include poly-L-lysine (PLL) and polyamidoamine (PAMAM) dendrimers.

Vector systems that promote CpG internalization into DCs to enhance delivery and its localization to TLR9 have been developed. The amine-rich polycation, polyethylimine (PEI) has been extensively used to condense plasmid DNA, via association with DNA phosphate groups, resulting in small, positively charge condensates facilitating cell membrane association and DNA uptake into cells (Godbey W. T., Wu K. K., and Mikos, A. G. J. of Biomed Mater Res, 1999, 45, 268-275; Godbey W. T., Wu K. K., and Mikos, A. G. Proc Natl Acad Sci USA. 96(9), 5177-81. (1999); each herein incorporated by reference). An exemplary method for condensing CpG-ODN is described in U.S. Patent Application No. US 20130202707 A1 published Aug. 8, 2013, the entire content of which is incorporated herein by reference. Consequently, PEI has been utilized as a non-viral vector to enhance gene transfection and to fabricate PEI-DNA loaded PLG matrices that promoted long-term gene expression in host cells in situ (Huang Y C, Riddle F, Rice K G, and Mooney D J. Hum Gene Ther. 5, 609-17. (2005), herein incorporated by reference).

CpG oligonucleotides can be divided into multiple classes. For example, exemplary CpG-ODNs encompassed by compositions, methods and devices of the present invention are stimulatory, neutral, or suppressive. The term “stimulatory” describes a class of CpG-ODN sequences that activate TLR9. The term “neutral” describes a class of CpG-ODN sequences that do not activate TLR9. The term “suppressive” describes a class of CpG-ODN sequences that inhibit TLR9. The term “activate TLR9” describes a process by which TLR9 initiates intracellular signaling.

Stimulatory CpG-ODNs can further be divided into three types A, B and C, which differ in their immune-stimulatory activities.

Type A stimulatory CpG ODNs are characterized by a phosphodiester central CpG-containing palindromic motif and a phosphorothioate 3′ poly-G string. Following activation of TLR9, these CpG ODNs induce high IFN-α production from plasmacytoid dendritic cells (pDC). Type A CpG ODNs weakly stimulate TLR9-dependent NF-κB signaling.

Type B stimulatory CpG ODNs contain a full phosphorothioate backbone with one or more CpG dinucleotides. Following TLR9 activation, these CpG-ODNs strongly activate B cells. In contrast to Type A CpG-ODNs, Type B CpG-ODNS weakly stimulate IFN-α secretion.

Type C stimulatory CpG ODNs comprise features of Types A and B. Type C CpG-ODNs contain a complete phosphorothioate backbone and a CpG containing palindromic motif. Similar to Type A CpG ODNs, Type C CpG ODNs induce strong IFN-α production from pDC. Simlar to Type B CpG ODNs, Type C CpG ODNs induce strong B cell stimulation.

Exemplary stimulatory CpG ODNs comprise, but are not limited to, ODN 1585 (5′-GGGGTCAACGTTGAGGGGGG-3′) (SEQ ID NO: 21), ODN 1668 (5′-TCCATGACGTTCCTGATGCT-3′) (SEQ ID NO: 22), ODN 1826 (5′-TCCATGACGTTCCTGACGTT-3′) (SEQ ID NO: 23), ODN 2006 (5′-TCGTCGTTTTGTCGTTTTGTCGTT-3′) (SEQ ID NO: 24), ODN 2006-G5 (5′-TCGTCGTTTTGTCGTTTTGTCGTTGGGGG-3′) (SEQ ID NO: 25), ODN 2216 (5′-GGGGGACGA:TCGTCGGGGGG-3′) (SEQ ID NO: 26), ODN 2336 (5′-GGGGACGAC:GTCGTGGGGGGG-3′) (SEQ ID NO: 27), ODN 2395 (5′-TCGTCGTTTTCGGCGC:GCGCCG-3′) (SEQ ID NO: 28), ODN M362 (5′-TCGTCGTCGTTC:GAACGACGTTGAT-3′) (SEQ ID NO: 29) (all InvivoGen). The present invention also encompasses any humanized version of the preceding CpG ODNs. In one preferred embodiment, compositions, methods, and devices of the present invention comprise ODN 1826 (the sequence of which from 5′ to 3′ is TCCATGACGTTCCTGACGTT, wherein CpG elements are underlined, SEQ ID NO: 22).

Neutral, or control, CpG ODNs that do not stimulate TLR9 are encompassed by the present invention. These ODNs comprise the same sequence as their stimulatory counterparts but contain GpC dinucleotides in place of CpG dinucleotides.

Exemplary neutral, or control, CpG ODNs encompassed by the present invention comprise, but are not limited to, ODN 1585 control, ODN 1668 control, ODN 1826 control, ODN 2006 control, ODN 2216 control, ODN 2336 control, ODN 2395 control, ODN M362 control (all InvivoGen). The present invention also encompasses any humanized version of the preceding CpG ODNs.

Vaccines that Attenuate Diseases of Pathogenic Immunity by Re-Directing the Immune System from a Th1/Th17 to T Regulatory Biased Immune Response

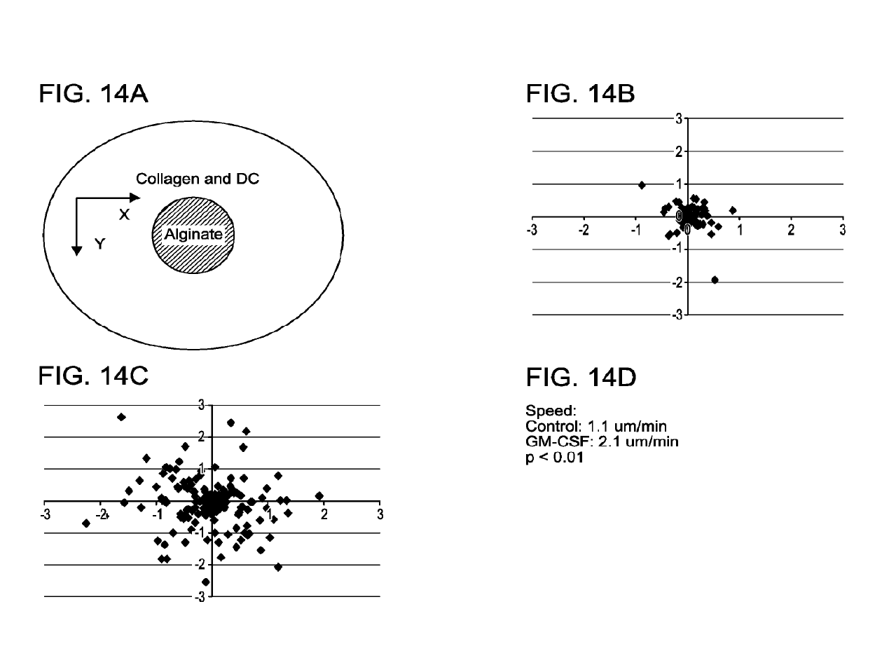

GM-CSF enhanced chemokinesis of bone marrow dendritic cells in vitro. Alginate gels with or without GM-CSF (˜1 g/gel) were placed in a petri dish and surrounded with collagen containing bone marrow derived murine dendritic cells (FIG. 14A). The cells were followed for 8 hours using time-lapse imaging. The velocity of the cells was calculated from initial and final position values and is plotted in FIGS. 14B and C in μm/min. Chemotaxis toward the alginate is given as the positive x coordinate (positive x is directed radially inward). Each dot reflects the velocity of 1 cell, and each plot is representative of three experiments. The average migration speed of cells in the presence of GM-CSF was 3.1 μm/min compared to 1.1 μm/min in the absence of GM-CSF. The speed of control and alginate gels is shown in FIG. 14D and was found to be significantly different at p<0.01. These data indicate that GM-CSF increases the speed of movement of dendritic cells and thus promotes dendritic cell migration.

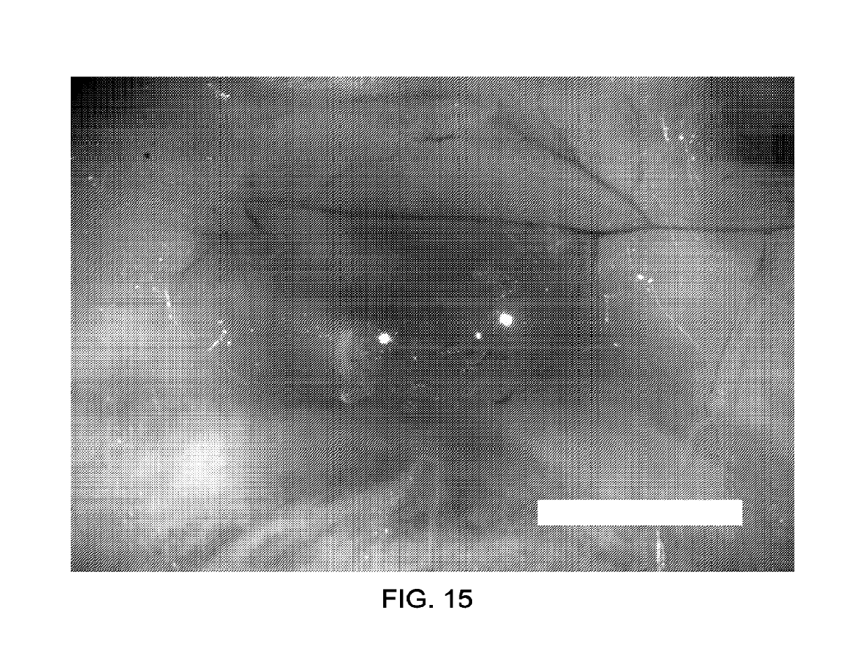

To observe the biomaterial scaffold in vivo, alginate gels were injected intradermally (FIG. 15). A 60 μL alginate gel was injected intradermally into the skin of a mouse. A photographic image was taken from the dermal side of the skin after euthanasia of the animal. Blue dye was incorporated into alginate gels before crosslinking for visualization.

### Recruitment of DCs to GM-CSF Loaded Alginate Gels In Vivo

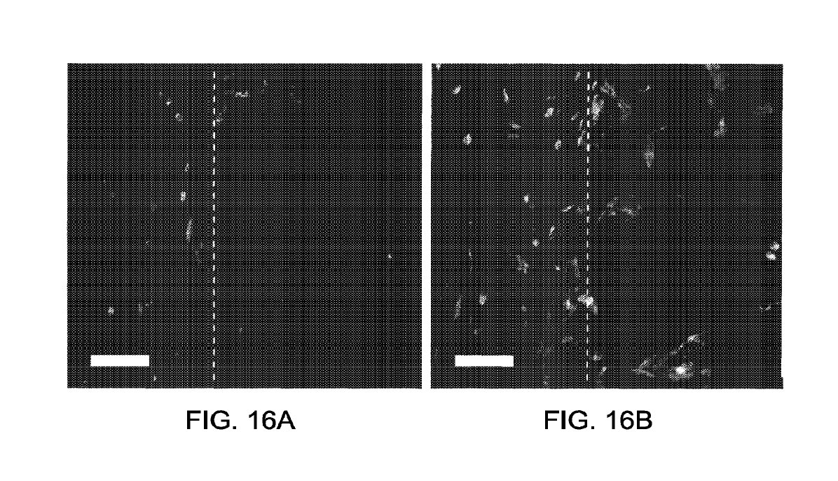

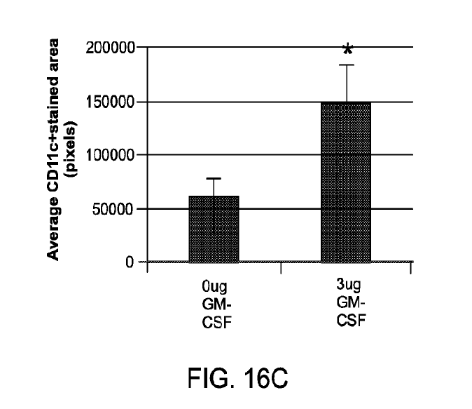

FIGS. 16A-B show the results of immunofluorescent staining of sectioned skin containing alginate gels, showing nuclei, MHC class II, and CD11c. Gels containing 0 μg (A) or 3 μg (B) of GM-CSF were explanted 7 days after injection. White dotted lines indicate the border between the dermal tissue (left) and the alginate gels (right). Scale bars are 50 μm. The area in tissue sections comprised of CD11c+ cells in blank gels vs. gels loaded with 3 ug of GM-CSF was quantified after 7 days. Image analysis of stained sections was done using ImageJ (n=3 animals/condition). *P<0.02. The data demonstrate that dendritic cells were recruited to GM-CSF loaded gels in vivo.

### T Regulatory (Treg) Cells are Recruited to GM-CSF/TSLP Loaded Gels

Treg cells were detected adjacent to alginate gels releasing GM-CSF and TSLP in vivo. TSLP promotes immune tolerance mediated by Treg cells and plays a direct and indirect role in regulating suppressive activities of such cells. The main influence of TSLP peripherally is on the DCs; however, T cells have receptors for TSLP and are also affected. Although Tregs are instrumental as being the mode of therapeutic benefit for periodontal disease, switch to a Th2 response (Th1->Treg/Th2) is also involved. For other diseases, a predominantly Treg response is desired; in the latter case, factors such as TGF-beta and IL-10 are utilized.

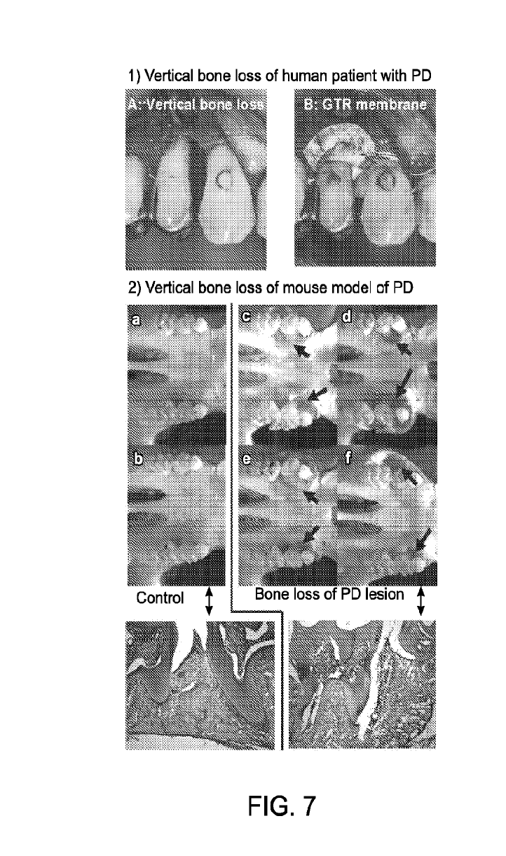

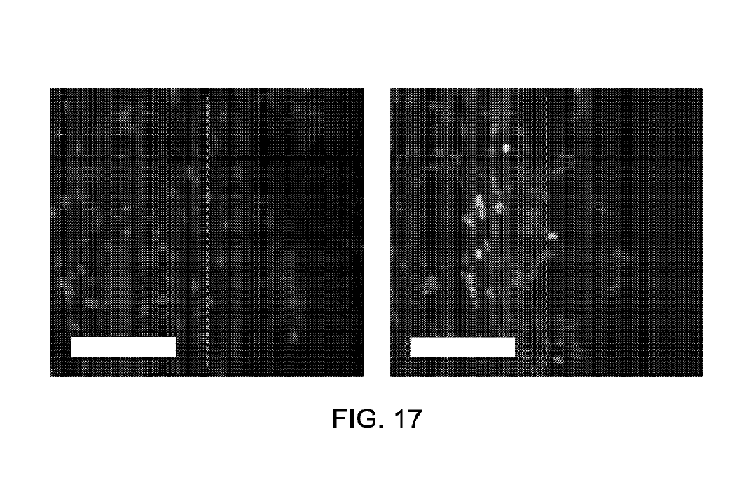

Cells were identified in FIG. 7 by detecting expression of FoxP3, a transcription factor specifically expressed in CD4+CD25+ Treg cells. Panels A and B of FIG. 17 show the results of immunofluorescent staining of sectioned skin containing alginate gels, showing nuclei (grey dots) and FoxP3 (bright dots). All gels contained 3 μg of GM-CSF. The gel in panel (A) did not contain TSLP (0 μg), whereas the gel in panel (B) contained 1 μg of TSLP. The gels were explanted 7 days after injection and analyzed. White dotted lines indicate the border between the dermal tissue (left) and the alginate gels (right). Scale bars are 50 m. Numerous bright dots (FoxP3-positive Treg cells) were detected using gels containing both GM-CSF and TSLP. These data indicate that in increased number of Treg cells are recruited to gels containing both GM-CSF and TSLP compared to GM-CSF alone or alginate alone.

### Dendritic Cell Immunotherapy for Type 1 Diabetes

The gel scaffolds described herein were evaluated in an art-recognized autoimmune model for type 1 diabetes mellitus (T1DM). The model utilizes a transgenic animal that expresses ovalbumin (OVA) under the control of the rat insulin promoter (RIP) in the pancreas (RIP-OVA model). (see, e.g., Proc Natl Acad Sci USA. 1999 Oct. 26; 96(22): 12703-12707; or Blanas et al., 1996. Science 274(5293):1707-9.). OVA-specific CD8-positive (cytotoxic T) cells are adoptively transferred intravenously to induce and establish autoimmune diabetes. More specifically, the adoptively transferred T cells recognize the ovalbumin presented on the pancreatic beta cells and attack these cells resulting in dampened insulin secretion and diabetes.

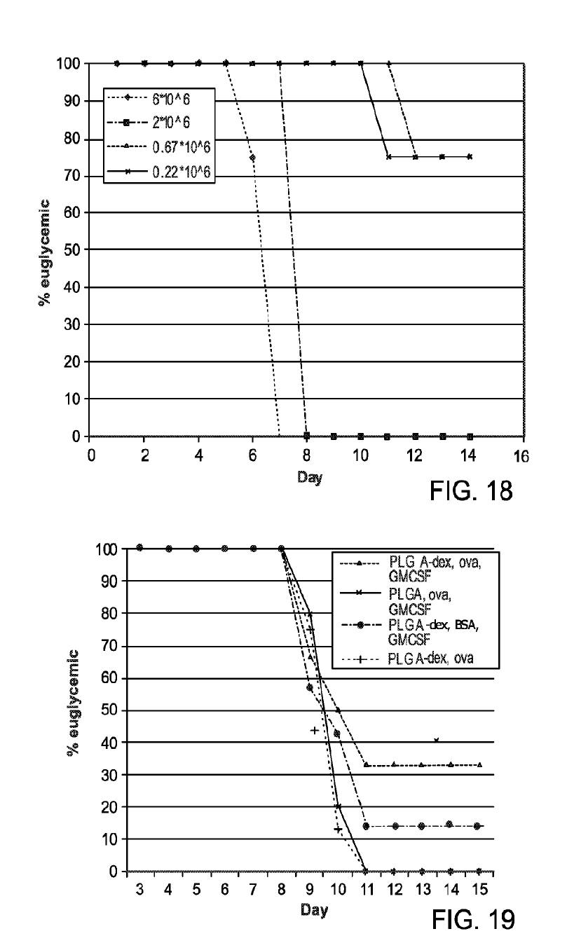

FIG. 18 shows percentages of euglycemic RIP-OVA mice over time following injection with various doses of OT-I splenocytes. 4 mice per group were injected with 6×10⁶, 2×10⁶, 0.67×10⁶, or 0.22×10⁶ activated CD8+Va2+OT-I splenocytes administered i.v. Adoptive transfer of approximately 2×10⁶ cells leads to diabetes in one week. Hyperglycemia was defined as 3 consecutive days with a blood glucose reading above 300 mg/dL. Between 0.67×10⁶ and 2×10⁶ T cells is a critical threshold for inducing disease. If cells are adminstered at this level concomitantly with therapies that influence T cell fate as described herein, the number the number of animals that eventually become diabetic and the speed at which they become diabetic is substantially altered in comparison to control animals with the adoptive transfer of cells alone without therapy.

Using the same model system, alginate gel scaffolds were implanted intradermally. The percentage of euglycemic mice was then determined over time following injection with 2×10⁶ OT-I splenocytes 10 days after alginate intradermal implantation (FIG. 19). All animals received an injection of alginate. Like TSLP, Dexamethasone (dex) is a composition that induces immune tolerance. In this experiment, dexamethasone was encapsulated in poly (lactide-co-glycolide) (PLG) microspheres prior to loading into alginate gels to delay release of the dexamethasone. The composition of the alginate gels was as follows: PLG: blank poly (lactide-co-glycolide) microspheres, PLGA-dex: dexamethasone (100 ng) encapsulated in poly (lactide-co-glycolide) microspheres, ova: ovalbumin (25 ug), GMCSF: granulocyte macrophage colony stimulating factor (6 ug), BSA: bovine serum albumin (25 ug). Hyperglycemia was defined as 3 consecutive days with a blood glucose reading above 300 mg/dL. Six or more mice were included in each group. Although dexamethasone blocks the action of insulin, a controlled spatio-temporal presentation of antigen+tolerogen led to an improvement in diabetes (greater percentage of euglycemic and slower onset of disease) in the PLGA-dex+Ova+GM-CSF group compared to the other groups, demonstrating that the combination of tolerogen, antigen, and recruiting agent in the context of a scaffold led to a reduction in a diabetes-associated autoimmune response specifically against pancreatic cells in vivo.

### Vaccines for Attenuation of Allergic Conditions

Immunoglobulin E (IgE) is a type of antibody that is normally present in small amounts in the body but plays a major role in allergic diseases. The surfaces of mast cells contain receptors for binding IgE. When IgE binds to mast cells, a cascade of allergic reaction can begin. IgE antibodies bind to allergens (antigens) and trigger degranulation and the release of substances, e.g., histamine, from mast cells leading to inflammation. Allergens induce T cells to activate B cells (Th2 response), which develop into plasma cells that produce and release more antibodies, thereby perpetuating an allergic reaction.

Scaffold-based vaccines were made to attenuate allergy, asthma, and other conditions characterized by aberrant immune activation by redirecting the immune system from a Th2 to a Th1 biased response. The scaffold-based vaccines reduced the production of IgE that leads to allergic symptoms caused by histamine (and other pro-inflammatory molecules) release due to mast cell degranulation.

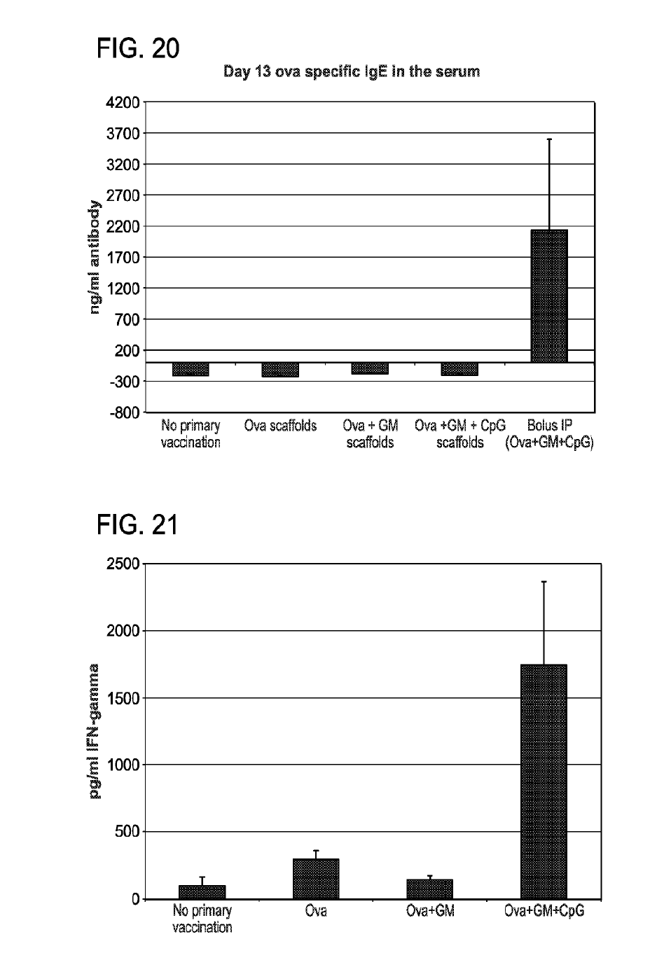

Antibody production in response to the vaccinations was first evaluated. Balb/c mice were left untreated (No primary vaccination control). Other mice were administered 10 μg of ovalbumin incorporated into a scaffold (Ova scaffolds), 10 μg of ovalbumin with 3 μg GM-CSF incorporated into a scaffold (Ova+GM scaffolds), 10 μg of ovalbumin with 3 μg GM-CSF and 100 μg CpG incorporated into a scaffold (Ova+GM+ CpG scaffolds), or 10 μg of ovalbumin with 3 μg GM-CSF and 100 μg CpG injected intraperitoneally (Bolus IP (Ova, GM, CpG). Poly lactide-co-glycolide (PLG) scaffolds were made by a gas foaming, particle leaching technique. 13 days later, the serum was collected from the animals and assayed by ELISA for ova-specific IgE antibody titres. The scaffold vaccines were administered subcutaneously into the flank. Bolus IP injection led to an IgE antibody response. However, scaffold mediated delivery of factors using scaffolds (i.e., using controlled release in a spatio-temporal manner) did not lead to an antibody response (FIG. 20). Therefore, the scaffold delivery strategy does not promote production of an allergic response mediated by IgE/mast cell degranulation.

On day 14, all of the mice were vaccinated with ovalbumin adsorbed to alum (adjuvant). 13 days later, serum ovalbumin-specific IgE was quantitated (day 27). N=5-10 animals. The mice were given Ova antigen+alum (adjuvant) to provoke a Th2-mediated allergic response. The data indicate that vaccination with scaffolds containing antigen+recruiting agent (GM-CSF)+Th1 promoting/stimulatory factor (CpG) reduces the Th2-mediated allergic response and preferentially increases the Th1-mediated response leading to reduction in allergy mediators.

The immune response elicited by the vaccines was further characterized. Balb/c mice were left untreated (No primary vaccination). Other mice were administered 10 μg of ovalbumin incorporated into a scaffold (Ova scaffolds), 10 μg of ovalbumin with 3 μg GM-CSF incorporated into a scaffold (Ova+GM scaffolds), or 10 μg of ovalbumin with 3 μg GM-CSF and 100 μg CpG incorporated into a scaffold (Ova+GM+ CpG scaffolds). 14 days later all of the mice were vaccinated with ovalbumin adsorbed to alum and 14 days later (day 28) the splenocytes from the animals were cultured with ovalbumin. Media was collected from the cell culture supernatants and IFN-gamma production or IL-4 production was assayed using an ELISA. N=5-10 animals. The results indicated that vaccination with all 3 factors in a scaffold (Ova+GM+ CpG scaffolds) led to an increased level of IFN-gamma, thereby demonstrating a shift toward a Th1 immune response (and away from a Th2 allergy response).

Bolus administration of CpG has sometimes been associated with splenomegaly. Experiments were therefore carried out to evaluate spleen enlargement following vaccine administration. The results indicated that bolus administration led to splenomegaly; however, delivery of factors (e.g., antigen/recruiting agent/Th1 stimulatory agent; Ova/GM-CSF/CpG) in a scaffold did not lead to splenomegaly. Thus, an advantage of the controlled spatio-temporal release of the factors from the scaffold is avoidance of the adverse side effect of spleen enlargement. The scaffolds and methods of using them have many other advantages compared to other strategies that have been developed to take advantage of the dendritic cell's central role in the immune system including antibody targeting of DC and ex vivo DC adoptive transfers. The former technique lacks specificity and unlike the scaffold poorly controls the microenvironment where antigen is detected. Adoptive transfer is costly, ephemeral, and many of the cells die or function poorly following administration. The scaffold system described here is less costly, directs cells through the lifetime of the implant (continuous vs. batch processing), and does not require ex vivo cell processing which leads to poor cell viability and hypofunctioning.

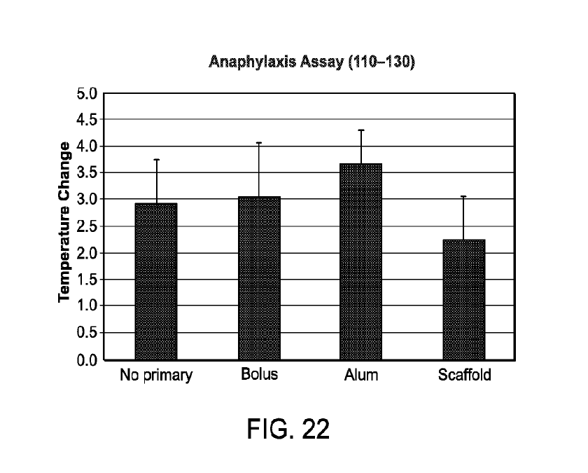

Vaccination was evaluated in an allergy animal model of anaphylactic shock caused by an antigen trigger. Histamine release leads to a change in temperature (decrease in temperature of the subject), which was used as a measure of the severity of allergic response. Balb/c mice were administered 10 μg of ovalbumin in alum (alum); 10 μg of ovalbumin with 3 μg GM-CSF, and 100 μg CpG subcutaneously (bolus); 10 μg of ovalbumin with 3 μg GM-CSF, and 100 μg CpG in a scaffold subcutaneously (scaffold); or no primary treatment (no primary) on day 0. On week 2, 5, and 8 the animals were vaccinated with ovalbumin adsorbed to alum and on week 11 the animals were administered 1 mg of ovalbumin intraperitoneally. n=7 or 8, error bars SEM. The results shown in FIG. 22 indicate that vaccination using a scaffold loaded with antigen+recruitment composition+adjuvant leads to a reduction in symptoms of allergy.

### Gel Scaffold Material Based Vaccines for Treatment of Periodontitis and Other Inflammatory Dental or Periodontal Conditions

Chronic inflammation is a major component of many of dentistry's most pressing diseases, including periodontitis, which is characterized by chronic inflammation that can lead to progressive loss of alveolar bone and tooth loss. Several tissue engineering and regeneration strategies have been identified that may be able to reverse the destructive effects of periodontitis, including the delivery of various morphogens and cell populations, but their utility is likely compromised by the hostile microenvironment characteristic of the chronic inflammatory state. The inflammation in periodontitis relates to both the bacterial infection and to the overaggressive immune response to the microorganisms, and this has led to efforts seeking to modulate inflammation via interference with the immune response. Therefore, there is an urgent need to devise novel therapeutic approaches for periodontitis treatment.

Chronic inflammation is characterized by continuous tissue destruction, and is component of many oral and craniofacial diseases, including periodontitis, pulpitis, Sjogren's, and certain temperomandibular joint disorders. Periodontal disease (PD), in particular, is characterized by inflammation, soft tissue destruction and bone resorption around the teeth, resulting in tooth loss. About 30% of the adult U.S. population has moderate periodontitis, with 5% of the adult population experiencing severe periodontitis. Also, because PD tends to exacerbate the pathogenicity of various systemic diseases, such as cardiovascular disease and low birth weight, PD can contribute to morbidity and mortality, especially in individuals exhibiting a compromised host defense. Guided tissue regeneration (GTR) membranes are commonly used to enhance periodontal regeneration, and these membranes provide a physical barrier to prevent epithelial cells from the overlying gingiva from invading the defect site and interfering with alveolar bone regeneration and reattachment to the tooth. GTR membranes can enhance regeneration, although typically not in a highly predictable manner, likely due to their passive approach to regeneration. Therefore, there is an urgent need to devise novel therapeutic approaches for PD treatment.

One of the major complications of periodontal diseases is the irreversible bone resorption that results in the loss of affected teeth. PD is treated currently by mechanical removal of the bacteria colonizing the teeth, and/or systemic or local antibiotic treatment. Although these approaches reduce the bacterial load can, when combined with appropriate oral hygiene, retard disease progression, they do not directly address the chronic inflammation driving tissue destruction nor promote regeneration of the lost tissue structures. Pathogenic bone loss in PD is induced by lymphocytes that produce osteoclast differentiation factor RANKL. One approach to preventing the progression of PD leading to bone loss is to modulate T- and B-cell responses to the bacterial infection in periodontal tissue. Using both rat and mouse models of PD, such an approach was indeed efficient in inhibiting immune-RANKL-mediated bone resorption. The methods and compositions described herein the chronic inflammatory response must be resolved to block further tissue destruction, and regeneration of the lost tissue must be promoted actively through inclusion of appropriate biologically active agents.

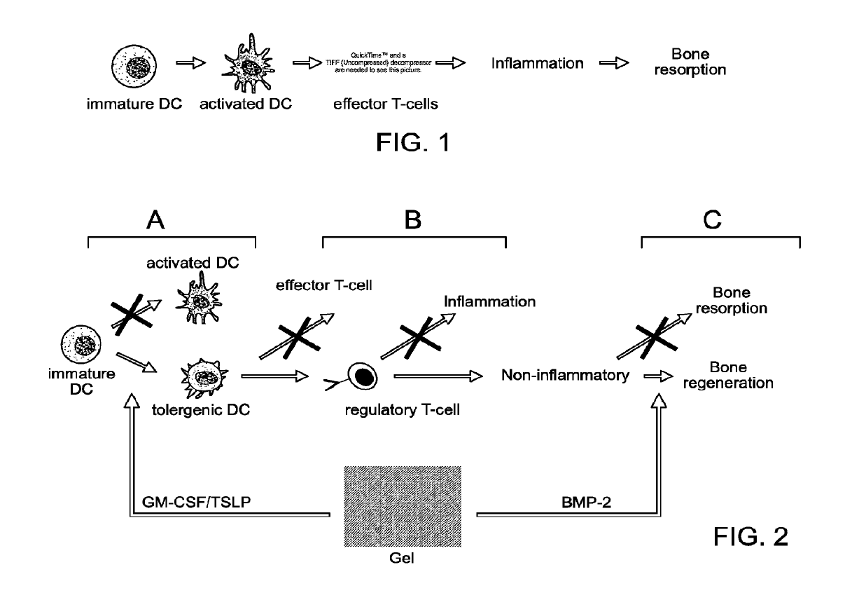

Aspects of the present subject matter relate to reducing periodontal inflammation and regenerating bone previously lost to PD. For example, the pathogenic process of bone resorption and inflammation elicited by lymphocytes (FIG. 1) is suppressed by FOXP3(+) T regulatory (Treg) cells via locally activated tolerogenic dendritic cells (tDCs). After the remission of inflammatory immune response by DC that promote the formation of regulatory T-cells (Tregs), the lost bone in the lesion is remodeled by localized delivery of a plasmid vector which encodes bone morphogenic protein (BMP). The material is administered using a minimally invasive delivery (i.e., gingival injection) and provides a temporally controlled release of functionally different bioactive compounds. The device promotes (a) initial DC programming to quench inflammation via recruitment and expansion of Tregs, and (b) subsequent release of a BMP-2 encoding plasmid vector to induce bone regeneration.

T-cells and B-cells play major role in bone resorption in PD in human and animal models. An active periodontal lesion is characterized by the prominent infiltration of B-cells and T cells. Specifically, plasma cells constitute 50%-60% of total cellular infiltrates, which makes PD distinct from other chronic infectious diseases. The osteoclast differentiation factor, Receptor Activator of NF-kB ligand (RANKL), is distinctively expressed by activated T-cells and B-cells in gingival tissues with PD, but not by these cells in healthy gingival tissues. The RANKL that was expressed on the T- and B-cells in patients' gingival tissues was sufficiently potent to induce in vitro osteoclastogenesis in a RANKL-dependent manner. The finding that RANKL is implicated as a trigger of osteoclast differentiation and activation in almost all inflammatory bone resorptive diseases emphasizes the importance of addressing this target.

Mouse models are recognized as the art for the study the roles of DCs and Tregs in bone regeneration processes in PD, in which inflammatory periodontal bone resorption is induced by the immune responses to live bacterial infection (FIG. 1). Adoptive transfer of antigen-specific T-cells or B-cells that express RANKL can induce bone loss in rat periodontal tissue that received local injection of the T-cell antigen A. actinomycetemcomitans (Aa) Omp29 or whole Aa bacteria as the B-cell antigen. The involvement of T-cells in the bone resorption processes was demonstrated by two inhibitors: (1) CTLA4-Ig (binding inhibitor for T cell CD28 binding to B7 co-stimulatory molecule expressed by APC); and (2) Kaliotoxin (blocker for T cell-specific potassium channel Kv1.3). Specifically, Kaliotoxin inhibits RANKL production by activated rat T cells. Adoptive transfer of an Aa-specific human T-cell line isolated from patients with aggressive (juvenile) periodontal disease could induce significant periodontal bone loss in NOD/SCID mice that were orally inoculated with Aa every three days.

Immune responses induced to Aa-immunized mice and rats do display Periodontal Pathogenic Adaptive Immune Response (PPAIR). Previous studies of rat models replicate most of the patho-physiological conditions of localized aggressive periodontitis (LAP) patients infected with Aa as well as some features of adult periodontitis. This model, relies on artificial bacterial antigen injection into gingival tissue rather than live bacterial infection. Furthermore, the lack of a variety of gene knockout rat strains hinders elucidation of the host genetic linkage to bacterial infection-mediated PD. A mouse model of PD replicates many of the critical features of human PD, and the pathogenic outcomes of adaptive immune reaction in mice, including those associated with RANKL induction, and is useful in terms of bone resorption induced in the periodontal tissue.

Tregs suppress overreaction of adaptive T effector cells and quench inflammation. Tregs were discovered originally as a subset of T-cells that showed suppression function in several experimental autoimmune diseases in animals. Tregs produce antigen-non-specific suppressive factors, such as IL-10 and TGF-β. In addition, they constitutively express cytotoxic T-lymphocyte antigen 4 (CTLA-4), which down-regulates DC activation and is a potent negative regulator of T-cell immune responses.

Anti-inflammatory effects mediated by Tregs also result from the up-regulation of extracellular adenosine, as Tregs convert extracellular ATP to this anti-inflammatory mediator via the action of CD39 and CD73. ATP released from injured cells or activated neutrophils is implicated as a danger signal initiator or natural adjuvant, because extracellular ATP promotes inflammation. Among all lymphocyte linage cells, only Treg are reported to express both CD39 and CD73, and can also suppress adenosine scavengers. Adenosine has various immunoregulatory activities mediated through four receptors. T-lymphocytes mainly express the high affinity A2AR and the low affinity A2BR. Macrophages and neutrophils can express all four adenosine receptors depending on their activation state, and B-cells express A2AR. Engagement of A2AR inhibits IL-12 production, but increases IL-10 production by human monocytes and dendritic cells, and selectively decreases some cytotoxic functions mediated by neutrophils. The primary biological role of Treg appears to be suppression of adaptive immune responses that produce inflammatory factors. Therefore, the ability to manipulate the formation and function of Tregs provides novel therapeutic approaches to a number of inflammatory immune-associated diseases, including PD (FIG. 2). Compared to generic anti-inflammatory drugs, which require frequent dosing, it is anticipated that once Tregs are generated in sufficient numbers, they could suppress inflammation induced by PPAIR not only in the acute phase, but also over extended time periods due to the immune memory function.

Tregs are identified via their expression levels of the transcription factor FOXP3. Patients with a mutated FOXP3 gene exhibit autoimmune polyendocrinopathy (especially in type 1 diabetes mellitus and hypothyroidism) and enteropathy (characterized as ‘immunodysregulation, polyendocrinopathy, enteropathy X-linked (IPEX) syndrome’). The similarity of the phenotypes between IPEX humans and Scurfy mice, which also show the FOXP3 gene mutation, suggests that FOXP3 mutation is a common cause for human IPEX and mouse Scurfy. FOXP3 gene variants (polymorphism) may also be linked to susceptibility to autoimmune diseases and other chronic infections. Importantly, FOXP3(+) cells are present in human gingival tissues, and, significantly, the expression level of FOXP3 appears to diminish in diseased gingival tissue compared to healthy gingival tissues. Even more importantly, FOXP3(+) T-cells do not express RANKL in the gingival tissues of patients who present with PD, indicating that FOXP3(+) T-cells are possibly engaged in the suppression of PPAIR. Furthermore, the Treg-associated anti-inflammatory cytokine, IL-10, is suppressed with the expression of sRANKL in human peripheral blood T cells stimulated in vitro by either bacterial antigen or TCR/CD28 ligation. Thus, FOXP3+ T-cells are implicated in the maintenance of periodontal health: (a) the diverse and exclusive expression patterns between RANKL and FOXP3 in the T-cells of human gingival tissue and (b) suppression of RANKL and other inflammatory cytokines produced by activated T-cells.

Treg cells limit the magnitude of adaptive immune response to chronic infection, preventing collateral tissue damage caused by vigorous antimicrobial immune responses. Because periodontal disease is a polymicrobial infection, it becomes relevant to elucidate how gingival tissue Tregs recognize such a huge and diverse variety of bacteria and, at the same time, regulate the adaptive effector T cells that also react to a vast number of bacteria. Several lines of evidence indicate that CD25(+)FOXP3(+)CD4(+) Treg cells are inducible from the CD25(−)CD4(+) adaptive T-cell population, especially in response to infection. These are often termed induced Treg cells (iTreg), and their induction, which is remarkably similar to the naturally-occurring Treg (nTreg) populations, is generated by peripheral activation, particularly in the presence of IL-10 or TGF-3. The diversity of T-cell receptors (TCRs) within the whole FOXP3(+) Treg population exceeds that of FOXP3(−)CD4 T cells. The presence of antigen-specific Treg has also been found in a variety of infectious diseases, including Leishmania, Schistosoma, and HIV. All these results are consistent with the mechanism that Treg recognize foreign antigens. Because periodontal disease is a polymicrobial infection, it becomes relevant to utilize Treg in suppressing the inflammation associated with the activated adaptive effector T-cells that also react to a vast number of bacteria.

The immune response (e.g., Treg induction) is orchestrated by a network of antigen-presenting-cells, and likely the most important of these cell types are DCs. Tissue-resident DCs routinely survey and capture antigen, and present antigen fragments to T-cells. The antigen presentation by DCs plays a key role in directing the immune response against the antigen to either immune activation or tolerance. In the healthy gingival tissue, immune tolerance against the oral commensal bacteria is induced, whereas immune activation is elicited to the periodontal pathogens in the context of PD, as demonstrated by elevated IgG antibody response to the periodontal pathogens, as described above. These two opposed outcomes, tolerance vs. activation, are controlled by the DCs present in the gingival tissue. Tolerance-inducing DCs (tDCs) are also called regulatory DCs. One method used by tDC to prevent immune activation is to generate iTreg cells during antigen presentation. The state of maturation and activation of DCs is critical to Treg development: DCs activated and maturing in response to inflammatory stimuli trigger immune responses, but immature or “semimature” DCs, in contrast, induce tolerance mediated by the generation of Tregs. The major phenotypic feature of tDC is their production of IL-10 and low or no production of IL-12 and other cytokines that prime effector T-cells. A number of signals and cytokines direct DC trafficking and activation. Multiple inflammatory cytokines mediate DC activation, including TNF, IL-1, IL-6, and PGE2, and are frequently used to mature DC ex vivo.

Granulocyte macrophage colony stimulating factor (GM-CSF) is a particularly potent stimulator of DC recruitment and proliferation during the generation of immune responses, and is useful to manipulate DC trafficking in vivo. A variety of exogenous factors including TGFß, thymic stromal lymphopoietin (TSLP), vasoactive intestinal peptide (VIP), and retinoic acid (RA), used alone or in combination, orientate DC maturation induce tolerance, and Treg development.

### Morphogens

A number of morphogens (e.g., bone morphogenetic proteins (BMPs), platelet derived growth factor (PDGF)) that actively promote bone formation by tissue resident cells are useful for prompting alveolar bone regeneration. The BMPs, members of the TGF-β superfamily, play a key role in that process. The BMPs are dimeric molecules that have a variety of physiologic roles. BMP-2 through BMP-8 are osteogenic proteins that play an important role in embryonic development and tissue repair. BMP-2 and BMP-7, the first BMPs to be available in a highly purified recombinant form, play a role in bone regeneration. BMP-2 acts primarily as a differentiation factor for bone and cartilage precursor cells towards a bone cell phenotype. BMP-2 has demonstrated the ability to induce bone formation and heal bony defects, in addition to improving the maturation and consolidation of regenerated bone. PDGF is a protein with multiple functions, including regulation of cell proliferation, matrix deposition, and chemotaxis, and has also been investigated for its potential to promote periodontal regeneration. PDGF delivery influences repair of periodontal ligament and bone, and ligament attachment to tooth surfaces. Recombinant proteins are used as the active agent in bone regeneration therapies. Alternatively local gene therapy strategies are used to deliver morphogen.

Sustained local production and secretion of growth factors via gene therapy overcomes certain limitations of protein delivery related to short half-life and susceptibility to the inflammatory environment, and also allows regulation of the timing of factor presence at a tissue defect site. Small-scale clinical trials and animal studies have documented success utilizing adenovirus gene delivery approaches, or transplantation of cell populations genetically modified in vitro prior to transplantation, to promote local expression of growth factors to drive bone regeneration. Delivery of plasmid DNA containing genes encoding for growth factors is preferred. Plasmid delivery requires large doses, and this results in expression of the transgene for about 7 days or fewer. Plasmid DNA delivery from polymer depots, increases transfection efficiency and duration of morphogen delivery.

### Delivery Systems

Programming of DCs and host osteoprogenitors in situ to generate potent, and specific immune and osteogenic responses involves precisely controlling in time and space a variety of signals that act on these cells. One approach to provide localized and sustained delivery of molecules at the desired site of action is via their encapsulation and subsequent release from polymer systems. Using this approach, the molecule is slowly and controllably released from the polymer (e.g., via polymer degradation), with the dose and rate of delivery dependent on the amount of drug loaded, the process used for drug incorporation, and the polymer used to fabricate the vehicle. In addition, polymer systems permit externally regulated release of encapsulated bioactive molecules e.g., using ultrasound as the external trigger. A variety of different polymers, and varying physical forms of the polymers have been developed to allow for localized and sustained delivery of various bioactive macromolecules. Biodegradable polymers of lactide and glycolide (PLG), which are also used to fabricate GTR membranes, are used clinically for extended delivery of hormones (Lupron Depot® microspheres [Takeda Chemical], and Zoladex microcylindrical implants [Zeneca Pharmaceuticals]. PLG microspheres that sustain the release of Macrophage Inflammatory Protein (MIP-3β) are chemoattractive for murine dendritic cells in vitro. Polymer rods have also been used to locally codeliver MIP-3β with tumor lysates or antigen, and induced the recruitment of dendritic cells that were able to induce antigen-specific, cytotoxic T-lymphocyte activity that yielded anti-tumor immunity.

Intratumoral injection of GM-CSF and IL-12 loaded microspheres was shown to generate protective immunity. Alginate-derived polymer, a depot system suitable has been used as carrier for immune regulating cues and osteogenic stimuli. Alginate is a linear polysaccharide comprised of (1-4)-linked β-D-mannuronic acid and α-L-guluronic acid residues, and is hydrophilic. Alginate gels promote very little non-specific protein absorption, likely due to the carboxylic acid groups, and has an extensive history as a food additive, dental impression material, and in a variety of other medical and non-medical applications. In the pure form, it elicits very little macrophage activation or inflammatory response when implanted Sodium salts of alginate are soluble in water, but will gel following binding of calcium or other divalent cations to yield gels that may readily be introduced into the body in a minimally invasive manner. These material systems have the ability to quantitatively control DC trafficking in vivo, and to specifically regulate DC activation. Such material systems provide control of host immune and inflammatory responses, while simultaneously providing signals that actively promote periodontal tissue regeneration.

### Chronic Inflammation in Periodontal Diseases (PD)

Chronic inflammation accompanying PD promotes bone resorption via involvement of immune cells (FIG. 1). Materials, hydrogels in particular, and therefore introduced into diseased tissue and first deliver signals to alter the balance of the immune response to ameliorate inflammation, and subsequently provide on-demand, localized delivery of pDNA encoding BMP-2. These compositions and methods lead to significant bone regeneration (FIG. 2). DCs are targeted as a central orchestrator of the immune system, are potent antigen-presenting cells. Other cell types may provide targets for immune modulation, and the strategies described herein are applicable to those cell types as well. This invention provides for material systems that program DCs in order to alter the balance between Tregs and effector T-cells to ameliorate chronic inflammation. The ability of Tregs to produce anti-inflammatory cytokines such as IL-10, and suppress adaptive immune responses makes them an attractive target to ameliorate chronic inflammatory processes. Material systems offer the opportunity to control more precisely the numbers, trafficking, and states of DCs and T-cells in the body, in combination with their ability to provide osteoinductive stimuli.

In another aspect of the invention, bone regeneration is promoted via an inductive approach that involves localized delivery of plasmid DNA encoding BMP-2. Local gene therapy is used to promote osteogenesis, and pDNA approaches in particular. The therapeutic system combines osteoinductive factor delivery with the active quenching of inflammation, and the externally-triggered release of the osteoinductive factor once inflammation is diminished. In particular embodiments, alginate hydrogels are used as the material platform. These gels are introduced into the body in a minimally invasive manner and have proven useful to deliver proteins, pDNA and other molecules, and regulate their distribution and duration in vivo. Alginate hydrogels are particularly useful for the ultrasound-mediated triggered release.

Further regarding the material system to recruit large numbers of host DCs and to effectively induce these DCs to a tolerant state (tDCs), GM-CSF are a cue to recruit DCs and TSLP pushes recruited DCs to the tDC phenotype. The GM-CSF is released into the surrounding tissue to recruit DCs, promote their proliferation, and generally increase the numbers of immature DCs, while appropriate TSLP exposure converts these cells to tDCs. The relation between local GM-CSF and TSLP delivery and tDCs, leads to generation of tDCs while minimizing the numbers of activated DCs.

One embodiment characterizes the action of GM-CSF and TSLP, and their delivery via alginate gels. GM-CSF is a potent signal for DC recruitment and proliferation, and the GM-CSF concentration is key to its ability to inhibit DC maturation and induce tolerance. TSLP generates tDCs due to its ability to initiate and maintain T-cell tolerance. A number of other factors have been identified that enhance formation of tDCs and Tregs, including vasoactive intestinal peptide, Vitamin D and retinoic acid, and these may be used alone or in combination with TSLP.

Materials containing the GM-CSF and TSLP with the appropriate spatiotemporal presentation to recruit and develop tDCs in situ were developed. The effects of continuous GM-CSF and TSLP exposure (10-500 ng/ml GM-CSF; 10-200 ng/ml TSLP) are described herein. FACS analysis and other analytic method used are to characterize DC population by deleting markers of maturation, e.g. MHCII, CD40, CD80 (B7-1), CD86 (B7-2), and CCR7, evaluating their secretion of cytokines (TNF-α, IL-6, IL-12, IFN-α, IL-10 tDC are identified by low levels of CD40, CD80, CD86, MHCII, and high level of IL-10). The effects of gradients of GM-CSF on cell recruitment are evaluated using a diffusion chamber.

Alginate gels with varying rheological/mechanical properties and degradation rates are created through control over the polymer composition, molecular weight distribution, and extent of oxidation. The alginate formulation used was binary alginate composed of 75% oxidized low molecular MVG alginate and 25% high molecular weight MVG alginate crosslinked with calcium. The scaffold composition allows the localized delivery of GM-CSF and TSLP. The release rates of GM-CSF and TSLP depends on the gel cross-linking and degradation rate, e.g., the gels provide sustained release for a time-frame ˜1-2 weeks. These molecules are incorporated directly into the gel during cross-linking, as documented previously for other growth factors and pDNA. If the release occurs too rapidly (e.g., gel depleted within 1-2 days), the release may be retarded by first encapsulating the factors in PLG microspheres, that are then incorporated into gels, such as alginate gels, during cross-linking. In this approach, release from the PLG particles regulates overall release, and this rate is tuned by altering the MW and composition of the PLG. The release rates of the GM-CSF and TSLP are analyzed in vitro using iodinated factors, following factor encapsulation. For example, GM-CSF is released over a period of 2 days to 3 weeks. The bioactivity of the released factors is confirmed using standard cell-based assays known in the art.

Gels are injected in the gingival tissue of mice at the site of alveolar bone loss (e.g., 1.5 μl).

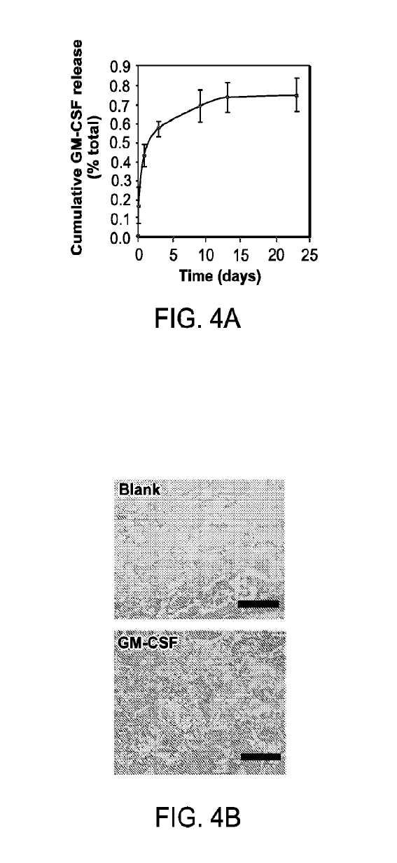

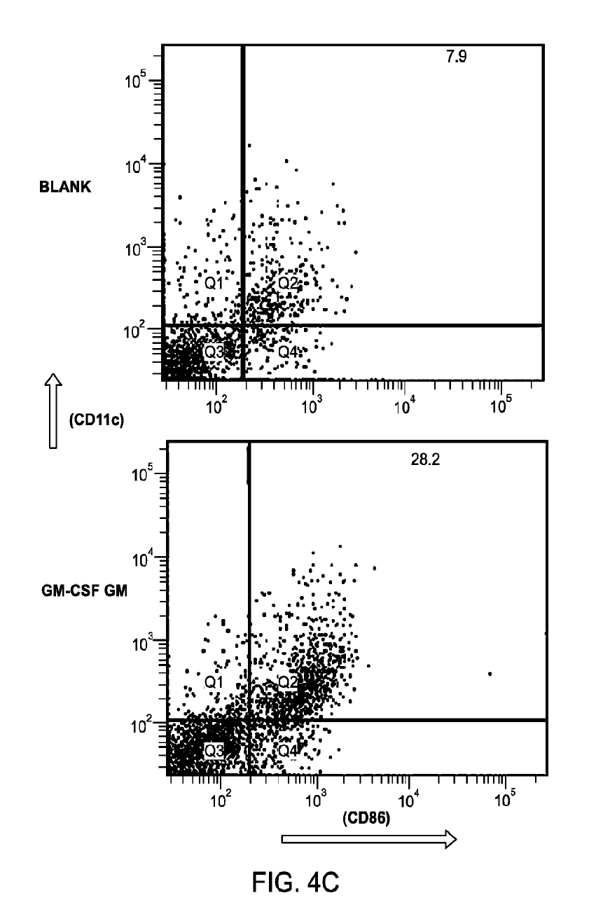

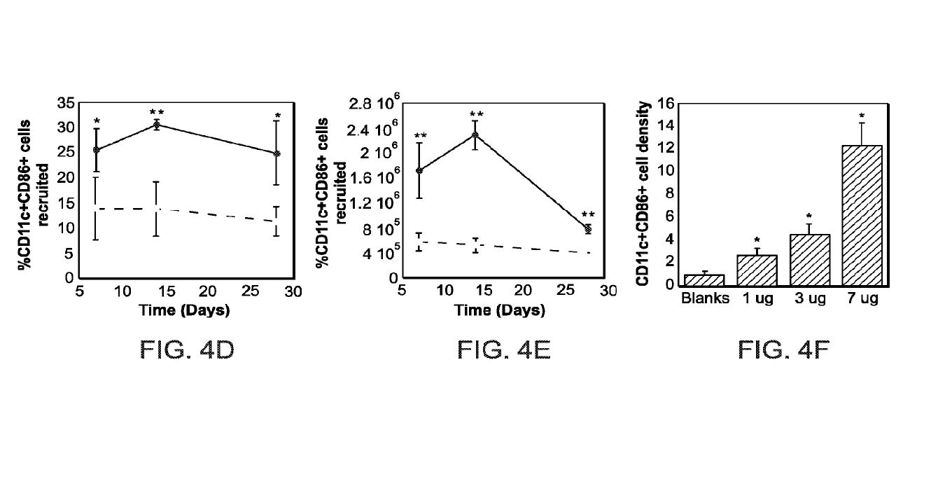

The ability of GM-CSF and TSLP to recruit host DCs (FIG. 4) indicates that an appropriate GM-CSF dose ranges from 200 ng-10,000 ng. The following factors were used to evaluate.

### Mouse Cytokine/Chemokine Panel-24-Plex

| Cytokine | Chemokine | Chemokine receptor(s) |
|---|---|---|
| TNF-a | Eotaxin | CCR3 |
| G-CSF | IP-10 | CXCR3, CXCR3B |
| GM-CSF | KC | CXCR2 |
| M-CSF | MCP-1 | CCR2** |
| IFN-γ | MIG | CXCR3 |
| IL-1ß | MIP-1a | CCR1, CCR5** |
| IL-2* | MIP-1ß | CCR5** |
| IL-4* | MIP-2 | CXCR2 |
| IL-6 | RANTES | CCR1, CCR3, CCR5** |
| IL-7* | | |
| IL-9* | | |
| IL-10 | | |
| IL-12 (p70) | | |
| IL-15* | | |
| IL-17 | | |

*γc-receptor-dependent cytokines
**reported to be expressed on Treg

Presentation of GM-CSF yields large numbers of recruited DCs, and a correlation between GM-CSF concentrations and DC maturation obtained (e.g., DCs maturation be inhibited at high GM-CSF concentrations). In other words, by controlling the release kinetics and dose of GM-CSF, it can act not only as a recruiting factor, but a tolerogenic factor. For example, at high concentrations of GM-CSF dendritic cells can become tolerogenic. If insufficient numbers of DCs are recruited with GM-CSF, exogenous Flt3 ligand release from gels is optionally used. TSLP is critical to direct the activation of DCs, particularly in the presence of inflammatory signals (e.g., LPS). The dose of TSLP relative to GM-CSF contributes to this phenomenon. For example, the range for each factor in a scaffold is 0.1 μg to 10 μg, e.g., scaffolds were made using 1 μg of each. TGF-beta, IL-10, rRetinoic acid, Vitamin D, and/or vasoactive intestinal peptide can optionally be added or used in place of TSLP. Alginate or PLG are preferred polymers; however other polymers and methods of TSLP and GM-CSF immobilization within the gels are known in the art.

Modulating PD-related inflammation with materials presenting GM-CSF and TSLP induces the formation of Treg cells and ameliorates inflammation in mice with PD. Inflammatory bone resorption found in human patients with PD was shown to be elicited by activated adaptive immune T-cells (and B-cells) which produce bone destructive RANKL as well as collateral inflammatory damage caused by expression of proinflammatory cytokines (IL-1-β, IFN-γ) from T-cells and other accompanying inflammatory cells. Suppressing the activation of T cells resolves the chronic inflammation and bone resorption associated with periodontal disease. Locally inducing anti-inflammatory Treg cells (iTregs) using the GM-CSF/TSLP material gel system shows tDCs generated by GM-CSF and TSLP formation of iTregs inhibit the inflammatory bone resorption induced by activation of adaptive immune responses. The level of inflammation is monitored by measurement of inflammatory chemical mediators present in gingival tissue (PGE₂, nitric oxide, ATP and adenosine) and presence of inflammatory cells.

Induction of tDCs in Periodontal Disease

The PD mouse model induces vertical periodontal bone loss following activation of immune responses to orally harbored bacteria, termed “Periodontal Pathogenic Adaptive Immune Response (PPAIR)”. Vertical bone loss is most closely associated with the human form of periodontal disease, and this PD model permits evaluation of: (1) inflammatory response by measurement of proinflammatory cytokines in the tissue homogenates; (2) localization and number of FOXP3+ Treg cells using FOXP3-EGFP-KI mice; (3) phenotypes of inflammatory cells by triple-color confocal microscopy and flow cytometry; (4) presence of bone destructive osteoclasts (TRAP), bone-generating osteoblasts (Periostin/alkaline phosphatase [ALP]), and ligament fibroblasts (Periostin/ALP); and (5) the level of bone resorption. Instead of a membrane-based GTR system, the selection of a gel-based delivery system is useful as a minimally invasive (non-surgical) material system to remodel vertical bone loss. More specifically, one gingival injection of gel appropriately delivers GM-CSF/TSLP. The socket wall at the vertical bone resorption lesion provides the space to retain the material, without the aid of a scaffold. After the successful demonstration of the principles underlying this approach, these gels are used as a supplement to current membrane-based GTR systems, or GTR systems that similarly provide these cues could be developed.

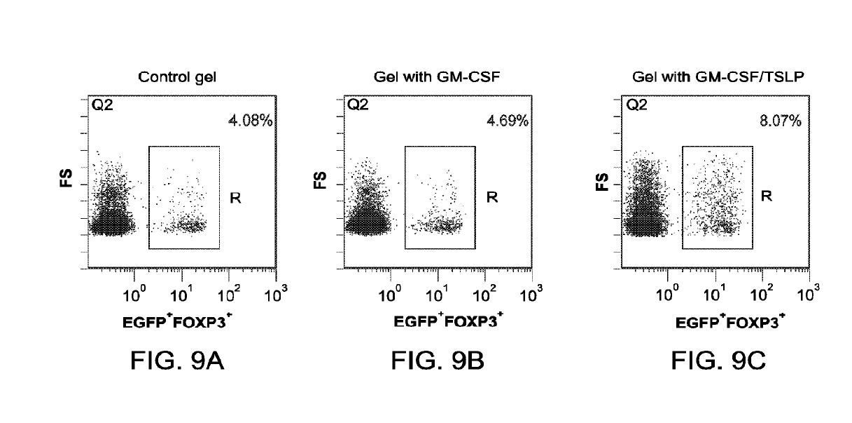

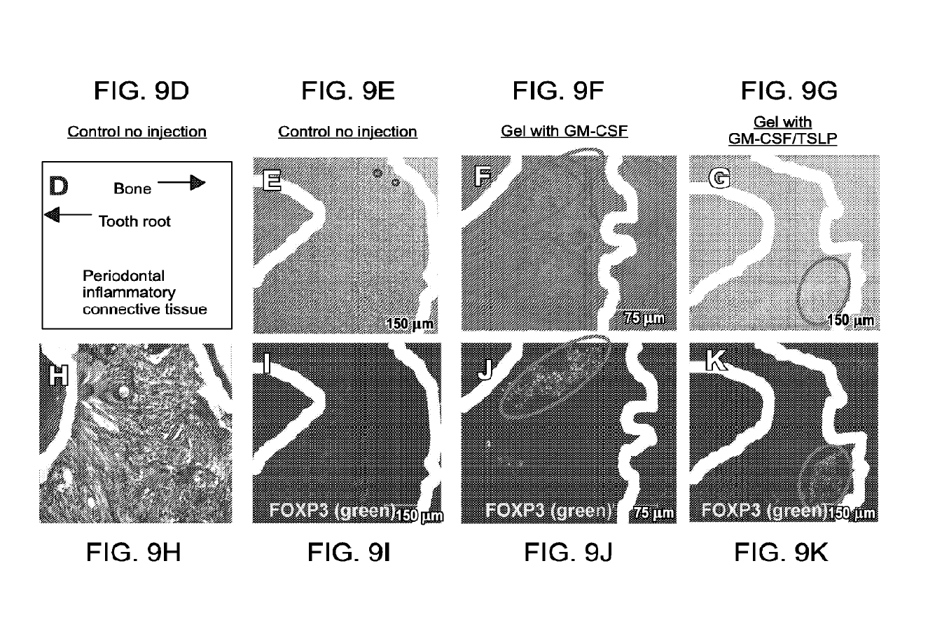

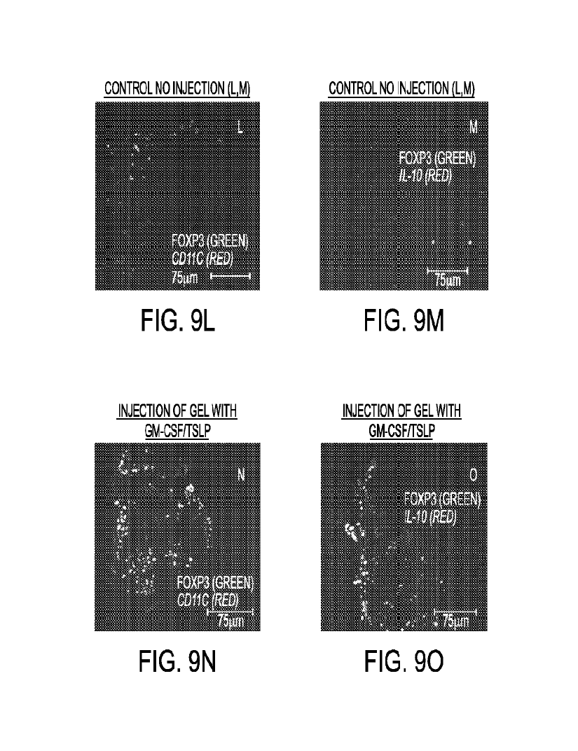

It is striking that increased numbers of FOXP3+ Treg cells were observed along with IL-10+CD11c+DC cells in the mouse periodontal bone loss lesion where GM-CSF/TSLP-gel was injected (FIG. 9). These data indicate that tDCs enhance local enrichment of (or promote generation of) FOXP3+ Treg cells. The GM-CSF/TSLP-delivered gel to induce tDCs. These aspects show the kinetics of iTreg induction by GM-CSF/TSLP delivery in alginate gels in periodontal bone loss lesions. The impact of the local formation of iTreg cells on the bone remodeling system (i.e., osteoclasts vs. osteoblasts and ligament fibroblasts) and continuation of bone resorption was observed.

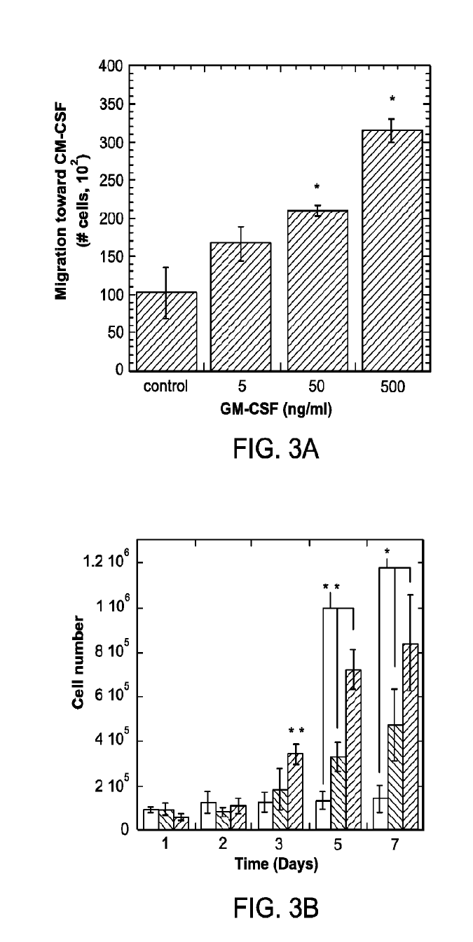

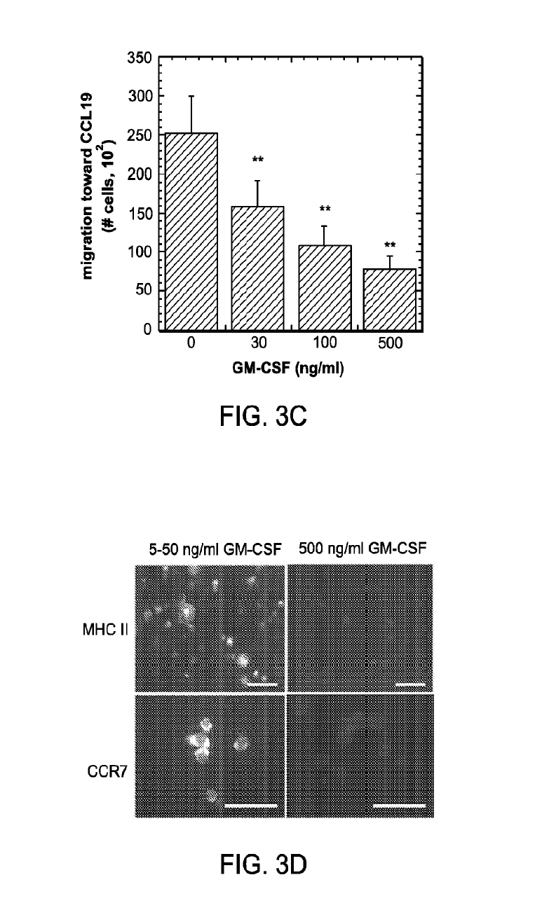

GM-CSF enhanced DC recruitment and proliferation in a dose-dependent manner (FIG. 3A-3B). High concentrations (>100 ng/ml) of GM-CSF, however, inhibited DC migration toward the LN-derived chemokine CCL19 (FIG. 3C). Immunohistochemical staining revealed that the high concentrations of GM-CSF also suppressed DC activation via TNF-α and LPS stimulation by down-regulating expression of MHCII and the CCL19 receptor CCR7 (FIG. 3D). These results indicate that local, high GM-CSF concentrations recruit large numbers of DCs and prevent their activation to a phenotype capable of generating a destructive immune response.

The GM-CSF/TSLP the recruitment of DCs and subsequent activation of iTregs, and provides local, material-based delivery of pDNA encoding osteogenic molecules in vitro leading to bone regeneration.

The polymer delivery vehicle presents GM-CSF in a defined spatiotemporal manner in vivo, following introduction into the tissue of interest. Exemplary vehicle quickly release approximately 60% of the bioactive GM-CSF load within the first 5 days, followed by slow and sustained release of bioactive GM-CSF over the next 10 days (FIG. 4A), to allow diffusion of the factor through the surrounding tissue and effectively recruit resident DCs. Polymers were loaded with 3 μg of GM-CSF and implanted into the dorsal subcutaneous site of C57BL/6J mice. Histological analysis at day-14 revealed that the total cellular infiltration at the site was significantly enhanced compared to control (no incorporated GM-CSF) (FIG. 4B). FACS analysis for CD11c(+)CD86(+) DCs showed that GM-CSF increased not just the total cell number, but also the percentage of infiltrating cells that were DCs (FIGS. 4C-4D). Enhanced DC numbers at the material-implanted site were sustained over time (FIG. 4E). As predicted by in vitro testing, the effects of GM-CSF on in vivo DC recruitment were dose-dependent (FIG. 4F).

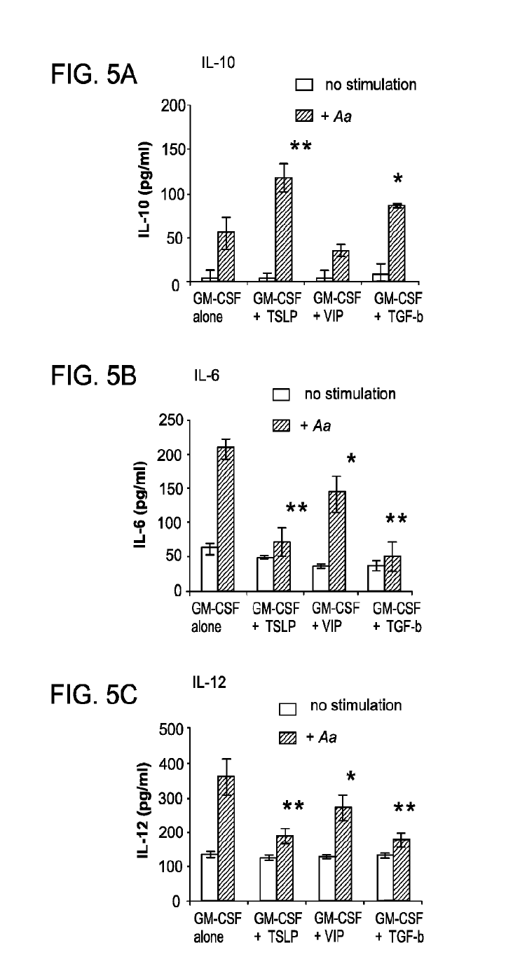

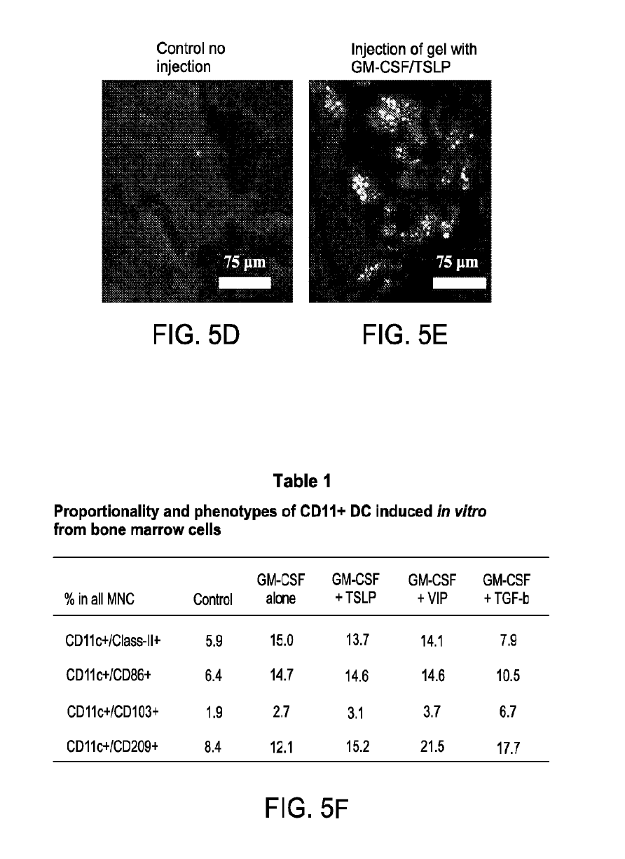

The present invention provides for a material-based local application of GM-CSF with appropriate DC influencing factors that leads to tolerogenic DCs (tDCs), and subsequent enrichment of iTreg cells. Candidate biofactors include thymic stromal lymphopoietin (TSLP), vasoactive intestinal peptide (VIP), and transforming growth factor-beta (TGF-β). Screening is based on the induced DC's anti-inflammatory properties. The in vitro incubation of mononuclear cells isolated from the bone marrow (BM) of C57BL/6 mice with GM-CSF in the presence of TSLP, VIP, or TGF-β led to diminished expression of the proinflammatory cytokines IL-6 and IL-12, in response to bacterial stimulation, as compared to the DC induced by GM-CSF alone (FIG. 5). In response to bacterial challenge, however, GM-CSF/TSLP-induced DC produced the highest levels of the anti-inflammatory cytokine, IL-10, as compared to the other combinations. Interestingly, the addition of TSLP did not alter the yield of GM-CSF-mediated differentiation of DC (CD11c+/CD86+ in total BM cells; GM-CSF alone, 14.7% vs. GM-CSF+ TSLP, 14.6%) from the BM cells compared to the low yield of CD11c+/CD86+DC with TGF-b (10.5%)(FIG. 5, Table 1). Overall, these observations that the combination of GM-CSF with TSLP efficiently induces DC with an anti-inflammatory phenotype.

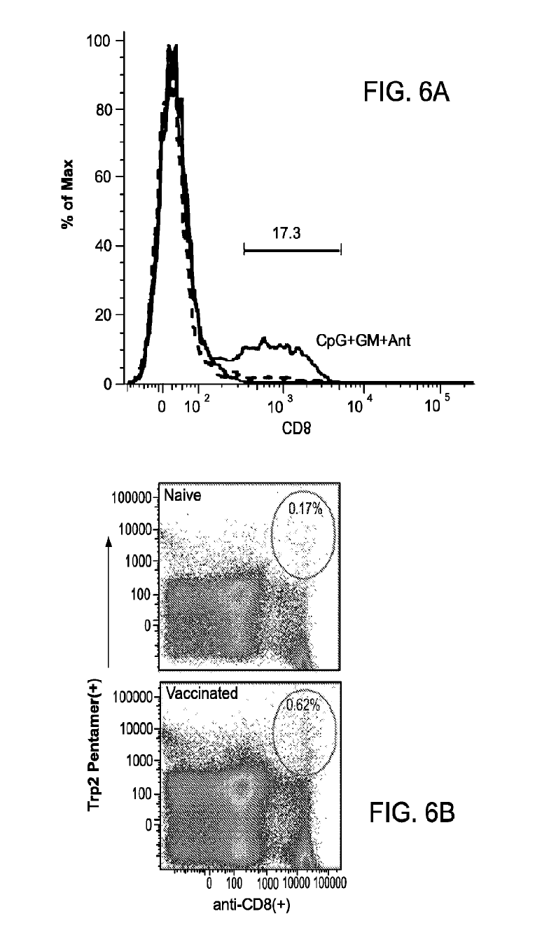

To demonstrate that material-based delivery of GM-CSF/TSLP induces tolerogenic DC locally in vivo, polymer vehicles containing a mixture of GM-CSF (1 μg) and TSLP (1 μg), as well as GM-CSF alone (1 μg), were injected into the periodontal bone resorption socket of FOXP3-EGFP-KI mice (C57BL6 background), and were evaluated to determine their effects on the local DC cells. Seven days later, a remarkable increase in the proportion of CD11c+IL-10+DC was observed in the periodontal socket of mice receiving polymers containing GM-CSF/TSLP, as compared to the injection of control empty polymer (FIG. 6). These findings indicate that the local delivery of TSLP and GM-CSF by the polymer can positively skew the GM-CSF-mediated differentiation of DC with anti-inflammatory activity, represented by high IL-10 expression, in the previously developed periodontal bone resorption lesion.

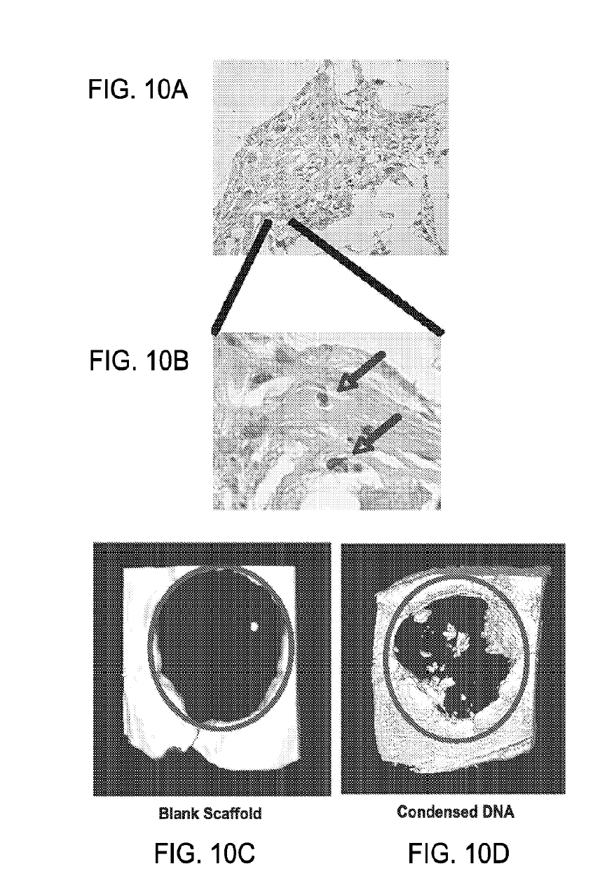

The ability of the material systems of the present invention not only to recruit DCs, but also to regulate T-cell generation, was also examined. These studies were performed to elicit an anti-tumor immune response against melanoma via inclusion in the material of “DC activators” (cytosine and guanosine-rich oligonucleotides; CpG-ODN; TLR9 ligand that elicits danger signal in DC, and melanoma-specific antigen, along with the GM-CSF. Nevertheless, although such approach “to activate immune response” contradicts to the approach “to suppress inflammatory-immune response,” the results demonstrate the ability to generate specific and quantitative immune responses with the material systems. Specifically, over 17% of the total cells at the site were CD8(+) compared to the control non-treated site (<1% CD8) (FIG. 6A). This result indicates that the number of T-cells infiltrating tissue adjacent to the polymeric delivery vehicle was enriched with delivery of GM-CSF, antigen and CpG-ODGN. The generation of a specific memory immune response was shown by staining isolated splenocytes with MHC class I/tyrosinase-related protein (TRP2). This analysis revealed a significant expansion of TRP2-specific CD8 T-cells in mice vaccinated with GM-CSF, antigen and CpG-ODN (0.55% splenocytes, 1.57×10⁵+5.5×10⁴ cells) in comparison to matrices presenting lower CpG doses, either 0 μg or 50 μg (0.17% and 0.25% of splenocytes) (FIG. 6B). As indicated above and in the next section (FIG. 10), the findings that the materials delivering GM-CSF and CpG oligonucleotides activate anti-tumor CD8 T-cells by activation of DC expressing IL-12, and in contrast when delivering GM-CSF and TSLP activate Treg cells by activation and differentiation of tolerogenic DC that produce IL-10, confirm the power of this approach to regulate immune responses.

The mouse model of PD was also used to study the efficacy of minimally invasive material systems that can suppress PPAIR, as well as induce regeneration in the bone loss lesion of PD, which meets the immuno-pathological fundamentals found in humans. This model develops RANKL-dependent periodontal bone loss upon induction of adaptive immune responses to the mouse orally colonized bacteria. By using the 16S rRNA sequence method, it was discovered herein that in-house bred BALB/c mice harbor the oral commensal bacterium Pasteurella pneumotropica (Pp). Pp is facultative anaerobic Gram(−) bacterium, and, similar to Aa, Pp is resistant to Bacitracin and Vancomycin, but susceptible to Gentamycin. Aa and Pp, as well as Haemophilus, belong to the same phylogenic family of Pasteurellaceae. Pp outer membrane protein OmpA is a homologue of Aa Omp29. Natural oral colonization of BALB/c mice with Pp per se is latent and has not shown any pathogenic features because immunological tolerance is induced to this oral commensal Pp. Supporting this, Pasteurella was also reported to be commensal in the gingival crevice of ferrets. Thirty days after either (1) adoptive transfer of the Aa-reactive Th1 line; or (2) peripheral immunization (dorsal s.c. injection) with fixed whole Aa to the Pp-harboring mice, periodontal bone loss (horizontal) was demonstrated, along with elevated IgG antibody response to Aa Omp29, and increased production of TNF-α and RANKL in the gingival tissue. The T-cells infiltrating in the gingival tissue expressed RANKL in the group of PD-induced mice, but not in the control group. Furthermore, systemic administration of OPG-Fc inhibited the periodontal bone loss induced in this mouse PD model, indicating that the induced periodontal bone loss is RANKL-dependent. The Aa immunization to the “Pp-free” BALB/c mice did not show periodontal bone loss, indicating that orally colonized commensal Pp bacteria that deliver the T-cell antigen to mouse gingival tissues is required for bone loss induction. Serum IgG of Aa-immunized Pp+ mice reacted to both Aa and Pp, but not other oral bacteria or E. coli examined. This very distinct cross-reactivity between Aa Omp29 and Pp OmpA allows the induction of Periodontal Pathogenic Adaptive Immune Response (PPAIR) that results in periodontal bone loss by immunization of Pp+ mice with Aa antigen. Indeed, Omp29 is one of the most prominent antigens recognized by serum IgG antibody in LAP patients infected with Aa.

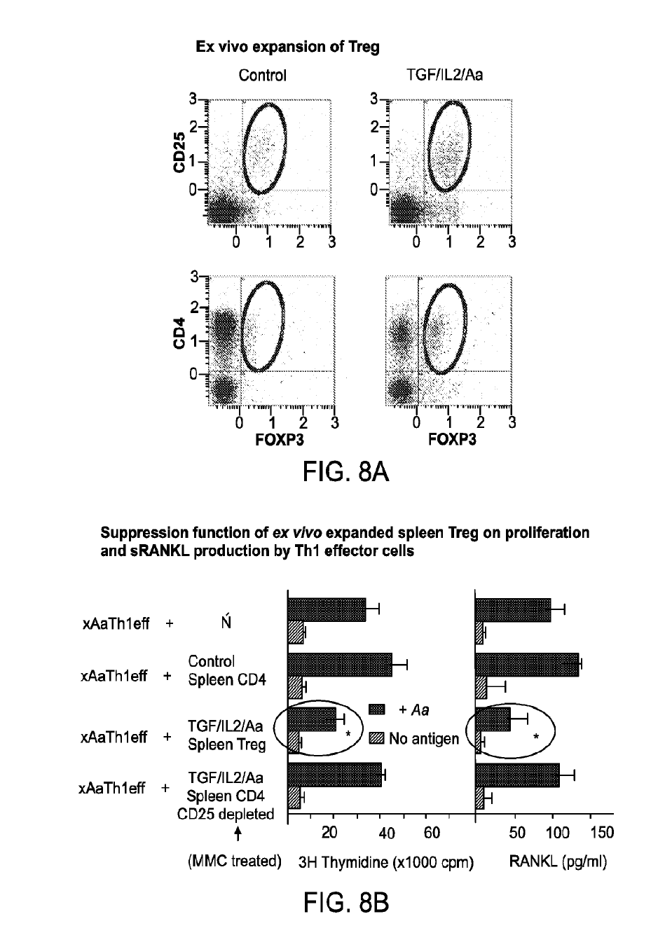

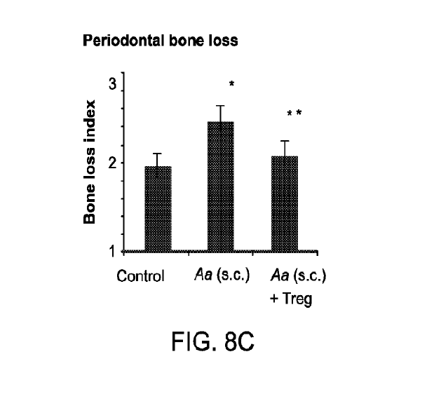

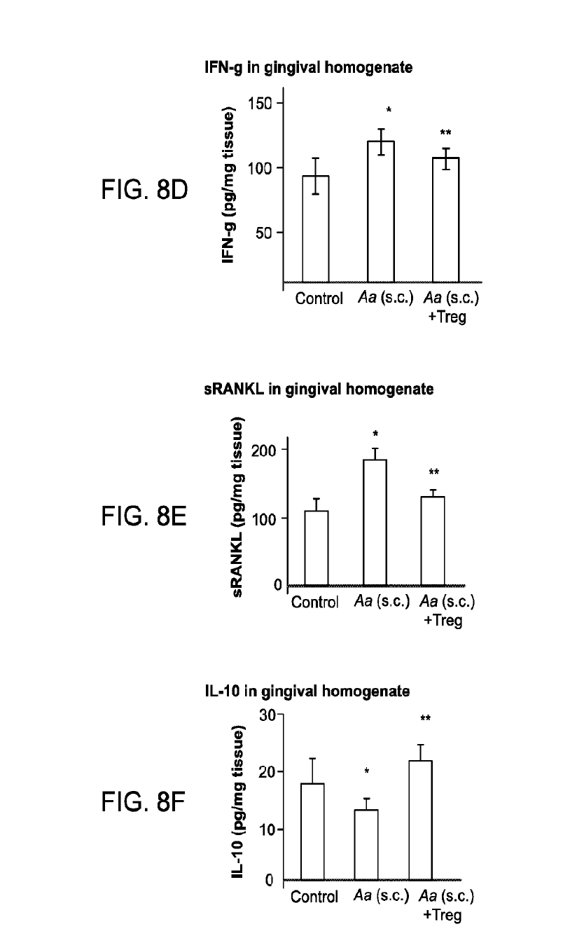

Although mouse models of P. gingivalis oral infection have been most frequently investigated, these P. gingivalis infection models appear to display mechanisms different from PPAIR. This occurs because induction of adaptive immune responses displayed by elevated IgG antibody to P. gingivalis antigen ameliorates, instead of augments, the P. gingivalis-infection-mediated periodontal bone loss, which is not necessarily representative of human periodontal bone resorption. Another shortcoming of the P. gingivalis-induced mouse PD model derives from the induction of only “horizontal periodontal bone loss,” while human PD is characterized by both “horizontal” and “vertical” periodontal bone loss. Although a number of etiological causes are proposed, horizontal bone loss is said to occur when chronic periodontal disease progresses moderately, while vertical bone loss is indicated when severe recurrent periodontitis or severe acute periodontitis progresses. The difference is important in the context of the proposed study because, while “horizontal” periodontal bone loss can be maintained by non-surgical periodontal treatment, “vertical bone loss” is, in fact, the clinical case where GTR surgery is required (FIG. 8).

Vertical periodontal bone loss with inflammatory connective tissue in mouse PD model, using the C57BL/6 strain mice, which followed the same protocol as published for BALB/c strain, demonstrated massive irreversible “vertical” periodontal bone loss (FIG. 7). This mirrors the periodontal bone loss found in most human patients with severe PD because, once having developed, vertical bone loss remains, even after the resolution of severe inflammation. For example, bone decay at the tooth extraction socket of mice is completely filled with new bone within 15 days. In contrast, vertical bone loss induced by PPAIR remains, indicating a significant difference in bone regeneration processes between bone loss caused by tooth extraction and by PD. It is noteworthy that few of the previously published animal models of PD develop vertical periodontal bone loss, and most of the periodontal bone loss induced in these animal models seems to develop horizontally and to be reversible after the resolution of inflammation. Therefore, this newly established mouse model provides the ideal platform with which to evaluate minimally invasive material systems that down-regulate inflammation as well as induce regeneration of lost bone. As illustrated in FIG. 7 (7g: control; 7h: PD lesion), the PD mice develop vertical bone loss filled with inflammatory connective tissue accompanied by TRAP+ osteoclast cells. Thus, minimally invasive material systems, such as the GM-CSF/TSLP delivery polymer described herein, can be administered to the inflammatory bone loss lesion such that both inflammatory response and bone regeneration in the bone loss lesion can be evaluated.

Adoptive transfer of FOXP3+CD4 T cells inhibits in vivo mouse bone resorption induced by PPAIR. In order to investigate if an increase of FOXP3+ Treg cells can suppress PPAIR-caused periodontal bone resorption, CD25+FOXP3+CD4+ iTreg cells were isolated from spleen T cells stimulated with TGF-b, IL-2 and Aa-antigen (FOXP3+CD25+ cells were 79.8% of the total CD4 T-cells) and were adoptively transferred to Pp+ BALB/c mice that were immunized with fixed Aa (dorsal s.c.) on Day-0, -2 and -4. In an in vitro assay, CD25+FOXP3+CD4+ iTreg cells suppressed the proliferation and production of RANKL by antigen/APC-stimulated Aa-specific Th1 effector cells (FIG. 8B). For control, non-immunized mice and Aa-immunized mice, without adoptive transfer, were prepared. Thirty days after Aa immunization, PPAIR was observed in the Aa-immunized mice, as determined by the elevated IgG1 responses to Omp29, elevation of IFN-γ and sRANKL in the local gingival tissue (FIGS. 8D and 8E), and periodontal bone resorption (FIG. 8C). The transfer of CD25+FOXP3+CD4+ iTreg cells to mice that received Aa systemic immunization significantly inhibited the following PPAIR features as compared to positive control animal groups: (1) increased IgG1 responses to Omp29; (2) IFN-g and sRANKL concentration in the gingival tissue (FIGS. 8D and 8E); and (3) local periodontal bone resorption (FIG. 8C). The amount of anti-inflammatory cytokine IL-10 in the gingival tissue was significantly increased by the transfer of iTreg cells (FIG. 8F). These results strongly suggest that local expansion of CD25+FOXP3+CD4+ iTreg cells can, in fact, inhibit periodontal inflammatory bone resorption induced by PPAIR by the mechanism of suppression of sRANKL and IFN-γ while activating IL-10 production in the local gingival tissues. This finding may be important in the context of the present invention because the efficacy of a material system in suppressing periodontal inflammation may be generated not by adoptive transfer, but by increasing host iTreg cells via activation of tolerogenic DC.

Local injection of polymer delivering GM-CSF/TSLP increases FOXP3+ T-cells in mouse gingival tissue and local lymph nodes (LN). The injection of polymeric delivery vehicles into the periodontal bone resorption socket of PD-induced FOXP3-EGFP-KI mice (C57BL6 background) was evaluated for the effects of the polymer on the resultant proportionality of Treg cells in the periodontal bone resorption lesion as well as local (cervical) lymph nodes. Seven days after the injection of polymer containing a mixture of GM-CSF (1 μg) and TSLP (1 μg) into the periodontal bone resorption socket (bone loss lesion developed 30 days after PPAIR induction by fixed Aa injection), an increase was observed in the proportion of FOXP3+EGFP+ Treg cells in cervical lymph nodes of mice that received GM-CSF/TSLP delivery polymer, whereas injection of polymer with GM-CSF (1 μg) alone did not show such increase of FOXP3+EGFP+ Treg cells in the local lymph nodes compared to the control empty polymer injection (FIG. 9). Interestingly, in the connective tissue of PD lesion, remarkable infiltration of FOXP3+ cells was observed in the mice receiving GM-CSF/TSLP-polymer, as well as GM-CSF-polymer, while few FOXP3+ cells were detected in the bone loss lesion of mice that did not receive any injection. Of interest, the FOXP3+ cells were found in foci that are composed of a number of inflammatory cell infiltrates, suggesting that the injected polymer may provide a scaffold for Treg cells to react with tolerogenic DC. To support this premise, the co-localization of FOXP3+ cells and tolerogenic DC was observed in the legion that received GM-CSF/TSLP-polymer (FIG. 9C). Therefore, the GM-CSF/TSLP polymer material delivery system demonstrably expanded the anti-inflammatory FOXP3+ Treg cells in periodontal bone resorption lesion as well as local lymph nodes.

Materials for localized pDNA delivery and tissue regeneration, and polymer systems for sustained pDNA release were developed to allow for the localized delivery and sustained expression of pDNA with kinetics dependent on the rate of polymer degradation. Macroporous scaffolds of PLG may be used for the encapsulation of pDNA, with its subsequent release regulated by the degradation rate of the particular PLG used for encapsulation; allowing for sustained release of plasmid DNA for times ranging from 10-30 days. To enhance the uptake of pDNA, and to localize the plasmid to the region encompassed by the polymer, pDNA was condensed with PEI prior to incorporation into the polymeric vehicles. Implantation of scaffolds containing either an uncondensed or PEI-condensed marker gene (luciferase) resulted in the short-term expression of the uncondensed DNA, but a very high and extended duration of expression for the PEI-condensed DNA. Further, implantation of polymers delivery PEI condensed pDNA encoding for BMP-2 or BMP-4 led to long-term BMP-4 expression by host cells (FIG. 10A), and significantly more bone regeneration than the polymer alone, delivery of non-condensed pDNA, or no treatment (FIG. 10B-10D).

This approach can be extended to injectable alginate gels. The degradation rate of alginate gels is altered by controlling the molecular weight distribution of the polymer chains comprising the gels. The rate of gel degradation (FIG. 11A) strongly correlated with the timing of release of PEI condensed pDNA encapsulated in the gels (FIG. 11B). The timing of pDNA expression in vitro and in vivo was regulated by the gel degradation rate, and this approach to pDNA delivery led to physiologically relevant expression in vivo of an encoded morphogen, and significant effects on local tissue regeneration.

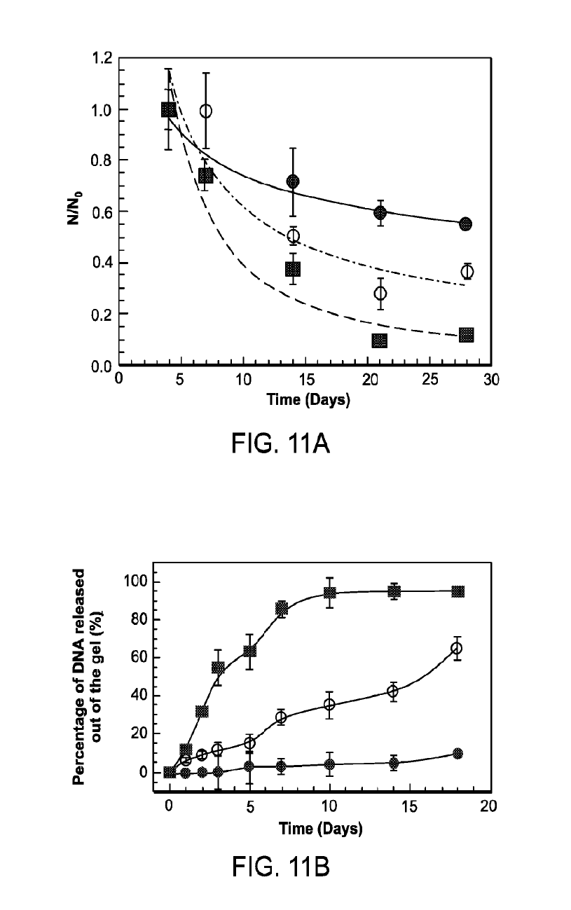

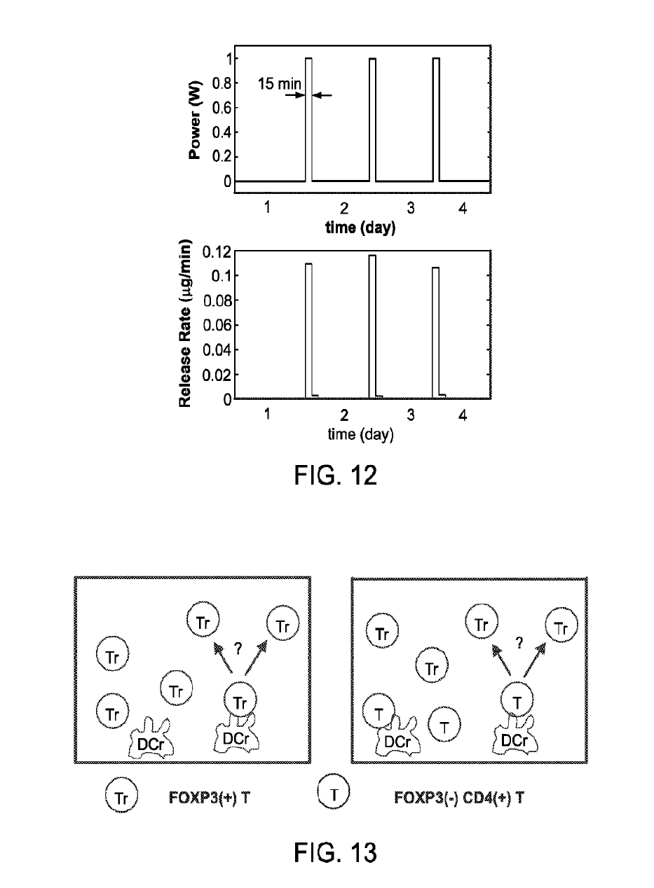

The present invention provides for the delivery of pDNA encoding an osteogenic factor subsequent to amelioration of chronic inflammation, using regulated pDNA release from the delivery vehicle. Ultrasound irradiation may be used to trigger the release of pDNA from alginate hydrogels, as ultrasound may provide an external trigger to control release of drugs from materials placed in periodontal tissue. Ultrasound has been pursued widely in past studies of drug delivery from the perspective of permeabilizing skin to enhance drug transport, but in present invention exploits the transient disruption of the gel structure during ultrasound application to enhance release of pDNA encapsulated in the gels. Use of a high molecular weight, non-oxidized alginate to form the gel (unary gel in FIG. 12A) led to minimal background release of pDNA, due to the slow degradation of this gel (FIG. 12). Application of appropriate ultrasound irradiation led to a 1000-fold increase in the pDNA release rate; the rate rapidly returned to baseline levels following cessation of irradiation (FIG. 12). The increase in pDNA release with ultrasound application correlated with large-scale perturbations of gel structure, as noted in past studies for biological samples. The subsequent rapid return of pDNA release rate to base-line levels correlated with a reversal of the gel structure to the original state. The ability of the alginate gels to “heal” following ultrasound likely is due to their reversible cross-linking with calcium ions in their environment. The present invention thus provides for precise control the timing of release of pDNA encoding osteogenic stimuli from the biomaterials matrix, at a time-point sufficient to first allow for conversion of the immune response to a non-inflammatory state.

### Analysis of Kinetics of Gingival Treg Cell Induction in the Mouse PD Model

Experiments were carried out to determine how long it takes for the induction of Treg cells and alterations in the local inflammatory environment with GM-CSF/TSLP delivery by alginate gel. Knowing the optimal time when inflammation is sufficiently and efficiently quenched by GM-CSF/TSLP-gel injection indicates the optimal timing for the release of pDNA-encoding BMP2 from the material system.

FOXP3-EGFP-KI mice (8 wk old, 12 males/group) that harbor Pp in the oral cavity receive immunization of fixed Aa (10⁹ bacteria/site/day dorsal s.c. injection on Day 0, 2 and 4). At Day-30, the development of periodontal bone loss is confirmed by probing of gingival pockets of maxillary molars. Serum IgG responses to Pp and Aa, along with the cross-reactive immunogenic antigens, including Pp OmpA (a homologue of Aa Omp29), are measured by ELISA because elevated IgG response to Pp antigens at Day-30 confirms that PPAIR successfully induces the development of vertical bone loss. Assuming that the levels of bone loss between left and right sides at Day 30 are symmetrical in each animal, the effects of GM-CSF/TSLP and the role of induced Treg cells are evaluated by palatal maxillary injection of gel with and without CD25+FOXP3+ Treg depletion by anti-CD25 MAb:

Group A: an injection of (1) mock empty gel to left, and 2) GM-CSF/TSLP to right, palatal maxillary gingivae;

Group B: same gingival injections as Group A, but the mice receive anti-CD25 MAb (500 μg/mouse, i.v. rat MAb hybridoma clone PC61 from ATCC) 3 days prior to gel injection;

Group C: same gingival injections as Group A, but the mice receive control purified rat IgG (500 μg/mouse, i.v.) 3 days prior to the gel injection;

Group D: an injection of mock empty gel to left, but no injection to the right, palatal maxillary gingivae.

The alginate gels were injected into the bone loss legion (1.5 μl/site). Animals are sacrificed on Day-33, -37, -44, and -58 (=3, 7, 14 and 28 days after injection of gels, respectively). Control, non-treated C57BL/6 mice sacrificed on Day-30 provide base-line information about inflammatory response and level of bone loss before the treatment with GM-CSF/TSLP-gel. The depletion of CD25+FOXP3+ Treg cells in Group B is confirmed by detection of CD25+FOXP3+ cells in the peripheral blood isolated from Group B and Group C using flow cytometry at Day-30. The dose and timing of TSLP/GM-CSF presentation from gels is determined, and 2-3 different doses are tested. Analysis included of: (1) Fluorescent immunohistochemistry for the detection of FOXP3+EGFP+ Treg cells and other inflammatory cell types (e.g., macrophages, neutrophils), gingival tissue cytokine measurement, detection of inflammatory chemical mediators in gingival tissue, and measurement of FOXP3+EGFP+ Treg cells and other lymphocyte phenotypes in cervical lymph nodes by flow cytometry; (2) analyses of TRAP+ osteoclasts, Periostin+/ALP+ osteoblasts and Periostin+/ALP+ ligament fibroblasts in decalcified periodontal tissues; and (3) extent of bone resorption using micro-CT, and quantitative histomorphometry.

Evaluation of Effects of GM-CSF/TSLP-Gels on the Immune Memory of iTreg Response

The efficacy of gel delivery of GM-CSF/TSLP in eliciting immune memory, as challenged by recurrent activations of PPAIR, was explored. The aspect of immune memory is significant because once immune memory of iTreg response can be induced, it should be capable of preventing recurrent episodes of pathogenic periodontal bone loss at the same site, and the development of future periodontal bone loss at different sites.

PD was induced as described above. At Day-30, Groups A and B receive identical gingival injections: (1) an injection of mock empty gel to left, and (2) an injection of GM-CSF/TSLP to right, palatal maxillary gingivae. At Day 44, however, Group A receives adoptive transfer of Aa/Pp cross-reactive Th1 cell transfer in saline (i.v.), as this has been shown to cause periodontal bone loss. Such Th1 cell transfer constitutes a secondary (recurrent) activation of PPAIR. Group B mice receive control saline (i.v.) injections. Animals are sacrificed on Day-51 (=21 days after injection of gels and 7 days after Th1 cell transfer). Control, non-treated C57BL/6 mice sacrificed on Day-30 provide the base-line information about inflammatory response and level of bone loss without treatment with GM-CSF/TSLP-gel. The analysis involves: (1) Fluorescent immunohistochemistry for the detection of FOXP3+EGFP+ Treg cells and other inflammatory cell types, measurement of gingival tissue cytokines and chemical mediators, and measurement of FOXP3+EGFP+ Treg cells and other lymphocyte phenotypes in cervical lymph nodes by flow cytometry; (2) Analyses of TRAP+ osteoclasts, Periostin+/ALP+ osteoblasts and Periostin+/ALP+ ligament fibroblasts in decalcified periodontal tissues; and (3) Periodontal bone loss measurement.

Relation Between tDCs and iTregs.

A series of studies addressed the relationship between GM-CSF/TSLP-induced tolerogenic DC (tDCs) and local development of Treg cells. The functional roles of chemokines and common γchain (γc)-receptor-dependent cytokines produced by GM-CSF/TSLP-induced tDCs on the extra-thymic development of Treg cells. Treg cells migrate to fungus-infected lesions in a CCR5 dependent manner in a mouse model of pulmonary mycosis, and Treg cells migrate to the infectious lesion in response to the CCR5-ligands, such as MIP-1α, which are also known to be expressed by GM-CSF-stimulated DC CD25+CD4+ Treg cells can be developed by ex vivo stimulation with TGF-β and IL-2 from whole spleen cells. Results (FIG. 8) demonstrated that ex vivo stimulation of mouse whole spleen cells with TGF-β and IL-2 up-regulated the development of FOXP3+ T-cells, indicating that FOXP3+ Treg cells are expandable ex vivo in response to appropriate stimulation. Common γchain (γc)-receptor-dependent cytokines are required for Treg cell expansion, which is demonstrated by the lack of Treg cells in γc-gene knockout mice. Several γc-receptor-dependent cytokines, e.g. IL-2, IL-7 and IL-15, up-regulate Treg development. Because TSLP, which also uses the γc-receptor, does not induce development of Treg cells TSLP released from the gels does not directly induce Treg development. However, DCs do not produce the major γc-receptor-dependent cytokine IL-2. Therefore, IL-15 that is produced by DC following stimulation with GM-CSF (Ge et all, 2002), facilitates Treg growth as a γc-receptor-dependent cytokine. If tDCs do not induce local development of FOXP3+ Treg cells from nTreg, then non-Treg cells, i.e., FOXP3(−)CD4(+) T cells, may migrate to the PD lesion and differentiate to FOXP3(+) iTreg cells by communication with the tDCs. Thus, these experiments examined in vitro chemokines and common γchain (γc)-receptor-dependent cytokines produced by GM-CSF/TSLP-induced tDCs and their functional roles in the chemo-attraction and development of FOXP3+ Treg cells.

Measurement of cytokines and chemokines produced by GM-CSF/TSLP-induced tDCs CD11+DC are induced in vitro by the incubation of bone marrow cells with GM-CSF (10 ng/ml) in the presence or absence of TSLP (10 ng/ml). After 7 days of incubation, CD11c+DC are isolated from the bone marrow cell culture, using anti-CD11c MAb-conjugated MACS beads (DC isolation kit, Miltenyi Biotech). CD11c+DC are be separated from mononuclear cells (MNC) freshly isolated from the dorsal s.c. tissue of mice where GM-CSF-gel, GM-CSF/TSLP-gel or control empty gel (GM-CSF and TSLP, 1 ug and 1 ug, respectively; 1.5 ul-gel/site) is injected 7 days prior to the MNC isolation, using anti-CD11c MAb-conjugated MACS beads. Doses and concentrations are adjusted as necessary. These DC are incubated in vitro in the presence or absence of bacterial stimulation (fixed Aa, fixed P. gingivalis, Aa-LPS or Pg-LPS) or proinflammatory factor (IL1-α), and their expression level of chemokines and cytokines is measured quantitatively by Mouse Cytokine/Chemokine Panel-24-Plex (Millipore; see Table 1) using a Luminex multiplex system. The production of inflammatory chemical mediators (PGE₂, NO, ATP, and adenosine) are also monitored, although detection of ATP and adenosine from DCs.

In vitro assays examined the Treg cell chemo-attractant factors secreted from DC. The culture supernatants of Aa- or IL-1-stimulated CD11c+DC, are placed in the bottom compartment of a transmigration system, while FOXP3(+)EGFP(+) Treg cells, or control FOXP3(−) CD4 T-cells, are freshly isolated from FOXP3-EGFP-KI mice by cell-sorting and applied to a cell-culture insert (5 μm pore size, Millipore). The kinetics and number of migrating FOXP3(+) Treg cells, or control FOXP3(−) CD4 T-cells, to the bottom compartment are monitored. In order to evaluate the functional role of Treg attracting factors, neutralizing mAb to the chemokines is applied to the bottom compartment with the supernatant of DC culture. MIP-1α is a Treg chemo-attractant secreted from tDCs. Recombinant chemokines serve as positive control chemo-attractant factors in this Treg cell migration assay. The expression of CCR2, CCR5 and other chemokine receptors expressed on the migrating FOXP3(+) Treg cells or control FOXP3(−) CD4 T-cells is monitored using flow cytometry.

In vitro assays examined the FOXP3+ Treg development factors secreted from DCs. The CD11c+DC were co-cultured with FOXP3(+)Treg cells and FOXP3(−) CD4 T-cells isolated from the spleens of FOXP3-EGFP-KI mice in the presence or absence of Aa-antigen. After 3, 7 and 14 days of incubation, the proportion of FOXP3(+)Treg cells are analyzed using flow cytometry. As can be observed from the scheme of possible results shown in FIG. 13, the advantage of using FOXP3-EGFP-KI mice with this assay system derives whether DC-mediated Treg development occurs from FOXP3(+)Treg cells or FOXP3(−) CD4 T-cells because: (1) live FOXP3(+)Treg cells can be isolated from FOXP3-EGFP-KI mice; and (2) development of mature Treg cells from their precursors, which do not express the FOXP3 gene, can be monitored by the detection of EGFP expression. In order to evaluate the functional role of Treg growth cytokines, neutralizing mAb to the cytokines are applied to the co-culture between DC and T-cells. IL-15 may be the major Treg growth cytokine secreted from tDCs.

Inflammation is suppressed in the PD lesion by 7 days (Day-37) after the injection of GM-CSF/TSLP-gel and that suppression effects lasts until Day-58, the latest examination day.

### Combining Anti-Inflammatory and Osteoinductive Signaling for Bone Regeneration

The utility of the immune programming system developed and studied is evaluated for its ability to enhance bone regeneration via co-delivery of osteoinductive cues. This approach both stops inflammation and actively promotes bone regeneration via delivery of pDNA encoding for BMP-2, using the same gel that releases GM-CSF/TSLP. The utility of the gel system is enhanced by its ability to release the pDNA on demand with an external signal (ultrasound irradiation). Ultrasound provides a number of advantages for this application, including its non-invasive nature, deep tissue penetration, and ability to be focused and controlled. The delivery system is used to first quench inflammation, and subsequently release pDNA to promote alveolar bone regeneration.

The first studies characterize ultrasound-triggered pDNA release from alginate gels, and subsequent studies examine bone regeneration using pDNA release from the gels in the PD model. Ultrasound can be used to trigger the release of pDNA from alginate gels after multiple days of incubation. Both PEI-condensed pDNA and uncondensed pDNA are encapsulated into alginate gels, and the passive pDNA release quantified. PEI-condensed pDNA is examined, as condensation dramatically upregulates pDNA uptake and expression, and the impact of ultrasound on release may be distinct for the two pDNA forms due to their different sizes and charges. Gels that vary in degradation times from 2-3 weeks to over 6 months are used for pDNA encapsulation, and little to no passive pDNA release occurs in the absence of gel degradation. The influence of varying regimes of low-frequency ultrasound irradiation (frequency of 20-50 kHz, intensity of 0.1-10 watt, duration 1-15 min) on pDNA release is examined after gels have incubated for times ranging from 1-3 weeks (to mimic the intended application in which GM-CSF/TSLP release occurs early and only following amelioration of inflammation will release of pDNA encoding BMP be triggered). The concentration of DNA in the release medium is assayed using Hoechst 33258 dye and a fluorometer (Hoefer DyNA Quant 200, Pharmacia Biotech, Uppsala, Sweden). The structural integrity of the released plasmid is examined using gel electrophoresis. Little effect of ultrasound on the GM-CSF and TSLP release is anticipated, as ultrasound is not initiated until after the majority of GM-CSF and TSLP have been released, but GM-CSF and TSLP release is be monitored during irradiation to determine if ultrasound impacts the release of any residual GM-CSF/TSLP remaining in the gels.

The ability of on-demand pDNA release from gels to enable in vivo transfection is examined to confirm both that ultrasound can regulate pDNA in vivo in a similar fashion as noted in vitro, and to determine the appropriate pDNA dose for bone regeneration studies. Gels containing pDNA encoding GFP are injected into palatal maxillary gingivae of normal mice (no periodontal disease), and subjected to ultrasound at times ranging from 7-21 days after introduction. The in vitro studies are used as a guide for the relevant frequency, intensity, and duration of irradiation. An exemplary ultrasound schedule comprises application once per day, for time-frames ranging from 1-7 days. One day following the end of each irradiation period, animals are sacrificed, and tissue sections obtained for both histology and biochemical quantification of overall GFP expression in the tissue. Uncondensed and PEI-condensed pDNA are compared in these studies, and the doses of encapsulated pDNA varied from 1 μg-100 μg. Tissue sections are immunostained for GFP to qualitatively study pDNA expression, and GFP levels also quantified in tissue lysates to quantify expression.

Another embodiment of this invention provides for the impact of the gel system to first ameliorate inflammation, and then actively promote regeneration in the PD mouse model. PD is characterized by chronic inflammation that leads to tissue destruction and bone resorption around the teeth. After induction of PD, gels containing GM-CSF, TSLP, and pDNA encoding BMP-2 are injected at Day-30. After sufficient time has elapsed to allow inflammation to reside, ultrasound irradiation is initiated to release pDNA encoding BMP-2. At 2, 4 and 8 weeks following gel placement, the soft and hard tissue is retrieved and analyzed. The level of inflammation is monitored by measurement of inflammatory chemical mediators present in the gingival tissue, and BMP-2 levels are also quantified with ELISA to examine gene expression. Bone regeneration is quantified using micro-CT and histologic analysis is also performed to allow quantitative histomorphometry of bone quantity. Controls include no treatment, gels containing pDNA only (no GM-CSF/TSLP), and blank gels. A sample size of 6/time point/condition is anticipated to be necessary studies of bone regeneration.

Reducing inflammation dramatically increases bone regeneration resulting from osteoinductive factor delivery, as compared to osteoinductive factor alone. Ultrasound provides a useful trigger to control the release of pDNA from alginate gels, both in vitro and in vivo, allowing a single gel to deliver the GM-CSF/TSLP and the plasmid with appropriate release kinetics. In some cases, there is an interplay between the gel degradation rate and ultrasound-triggered release due to the changes in gel structure resulting from degradation. Two gel injections—the first delivering GM-CSF/TLSP to ameliorate inflammation, and the second to delivery pDNA encoding BMP-2 after inflammation has been reduced, may be used.

High, local levels of BMP-2 significantly enhance bone regeneration. The major effect of ultrasound on regeneration is triggered release of pDNA from gels, but ultrasound also enhances cellular uptake of pDNA and thus directly enhances expression of locally delivered pDNA in addition or without effects on pDNA release.

The following materials and methods were used in periodontal studies described herein.

### In Vitro DC Assays

Migration assays are performed with 6.5 mm transwell dishes (Costar, Cambridge, Mass.) with a pore size of 5 μm. The effects of GM-CSF and TSLP, (Invivogen, San Diego, Calif.) on the migration of DCs are assessed by placing recombinant murine GM-CSF and TSLP in the bottom wells and 5×10⁵ DCs in the top wells. To assess the effects of GM-CSF and TSLP on DC activation, cells are cultured with bacterial stimulation (fixed Aa, fixed P. gingivalis, Aa-LPS or Pg-LPS along) with various concentrations of TSLP and GM-CSF for 24 hours and then the cells are washed and fixed in 10% formalin. The cells are prepared for fluorescence immunohistochemistry as per below, and examined using fluorescent microscopy (Olympus, Center Valley, Pa.). Cells are also analyzed by FACS, and gated according to positive stains using isotype controls, and the percentage of cells staining positive for each surface antigen will be recorded. The expression of cytokines upregulated as a result of DC maturation is quantified as described below.

### Gel Fabrication

Gels are created from alginates varying in mannuronic to guluronic acid residues, molecular weight distributions, and extent of oxidation to regulate their rheological, physical and degradation properties. Hydrogels are prepared by mixing alginate solutions containing the factors as previously described for proteins and plasmid DNA formulations with a calcium sulfate slurry. If necessary, factors are first encapsulated into PLG microspheres using a standard double emulsion technique.

Quantification of GM-CSF, TSLP, and pDNA In Vitro Release Studies, and In Vivo Concentrations

To determine the efficiency of GM-CSF, TSLP, and pDNA incorporation and the kinetics of release, ¹²⁵I-labeled factors (Perkin Elmer) are utilized as a tracer, and gels and placed in Phosphate Buffer Solution (PBS) (37° C.). At various time points, the PBS release media is collected and amount of ¹²⁵I-factor released from the scaffolds is determined at each time point using a gamma counter and normalizing to the total ¹²⁵I-factor incorporated into the gels. To asses the retention of GM-CSF bioactivity, loaded gels are placed in the top wells of 6.5 mm transwell dishes (Costar, Cambridge, Mass.) with a pore size of 3 μm and the proliferation of JAWS II cells (DC cell line) cultured in the bottom wells is evaluated at various time points using cell counts from a hemacytometer. To determine GM-CSF and TSLP concentrations in vivo, tissue surrounding gels is excised and digested with tissue protein extraction reagent (Pierce). After centrifugation, the concentration of GM-CSF and TSLP in the supernatant is then analyzed with ELISA (R&D systems), according to the manufacturers instructions.

### In Vivo DC Migration and Activation Assays

Gels with various combinations of factors are injected into gingival of mice. For histological examination gels and surrounding tissue are excised and fixed in Z-fix solution, embedded in paraffin, and stained with hematoxylin and eosin. To analyze DC recruitment, gels and surrounding tissue are excised at various time-points and the tissue digested into single cell suspensions using a collagenase solution (Worthingtion, 250 U/ml) that was agitated at 37° C. for 45 min. The cell suspensions are then poured through a 40 mm cell strainer to isolate cells from gel particles and the cells are pelleted and washed with cold PBS and counted using a Z2 coulter counter (Beckman Coulter). The resultant cell populations are then stained with primary antibodies conjugated to fluorescent markers to allow for analysis by flow cytometry. Cells are gated according to positive labels using isotype controls, and the percentage of cells staining positive for each surface antigen is recorded.

### Fluorescent Immunohistochemistry

To evaluate the tissue localization pattern of specific cells in gingival tissues and cervical LN, confocal microscopic analysis is employed. Using the 3-color staining procedure, key subsets, tDCs (cells positive for CD11c and CD86 and IL-10), mature DCs (positive for CCR7, B7-2, MHCII), FOXP3+ T cells (EGFP, IL-10 and TGF-b), FOXP3+CD25+ T cells (EGFP, CD25, IL-10), RANKL+CD3+ T cells (RANKL, CD3 and TNF-α) and RANKL+CD19+ B cells are stained. Expression of CD26, CD39 and CD73 on FOXP3+ T cells as well as on RANKL+CD3+ T cells, DC (CD11c+), B cells (CD19+), macrophages (F4/80+) and neutrophils (CD64+) are also monitored. Detection of RANKL is conducted by a combination of biotin-conjugated-OPG-Fc/TR-avidin. Other molecules are stained using a conventional method with primary specific-monoclonal antibody followed by secondary antibody conjugated with fluorescent dye: 1st color, FITC (emission/excitation, 488/515 nm); 2nd color, Texas Red (595/615); and 3rd color, APC/Cy5.5 (595/690).

### Flow Cytometry

The prevalence of various cells in gingival tissue and local cervical lymph nodes is analyzed by flow cytometry. Nonspecific antibody binding to the Fc receptor is blocked by pre-incubating the cells with rat MAb 2.4G2 (reactive to CD16/CD32). Three-color staining method is employed for the detection of tDCs, mature DC, EGFP+FOXP3+ T cells and RANKL+CD3+ T cells.

Detection of Cytokines from Culture Medium and Gingival Tissue Homogenates

Standard methods were used to detect cytokines and other markers such as IL-10, RANKL, OPG, Osteocalcin, TNF-α, IFN-γ, TGF-b1, IL-1b, IL-2, IL-4, IL-6, IL-12 and IL-17 in the culture medium or mouse gingival tissue homogenates.

### Detection of Inflammatory Chemical Mediators Present in Gingival Tissue

Both pro-inflammatory (PGE₂, nitric oxide [NO] and ATP) and anti-inflammatory chemical mediators (adenosine) are measured. PGE₂ is measured using a Luminex-based PGE₂ detection kit (Cayman Chemical). Nitric oxide present in tissue homogenate is measured by Nitrate/Nitrite Colorimetric Assay Kit (Cayman Chemical). The concentration of ATP and adenosine will be measured using Sarissaprobe®-ATP and Sarissaprobe®-ADO sensors (Sarissa Biomedical, Coventry, UK).

### TRAP Staining for Osteoclasts and Periostin/ALP Staining for Osteoblasts and Periodontal Ligament Fibroblasts in Periodontal Bone

The maxillary jaws of animals sacrificed on Day-33, -37, -44, and -58 are decalcified, and osteoclast cells determined by TRAP staining on the tissue sections. The tissue sections are also stained for Periostin and alkaline phosphatase to determine the localization of osteoblasts and periodontal ligament fibroblasts.

pDNA Studies

Plasmid DNA containing the CMV promoter and encoding for green fluorescent protein (GFP) (Aldevron, Fargo N. Dak.) or bone morphogenetic protein 2 (BMP-2) (Aldevron) are used. Branched polyethylenimine (PEI, MW=25000, Sigma-Aldrich) is used to condense plasmid DNA for more efficient transfection.

### Application of Ultrasound

An Omnisound 3000 will be to mediate pDNA release from gels. The structure of gels subject to sonication in vitro are examined via analysis of rheological properties at varying times post-treatment to determine permanent changes in gel structure, and recovery time post-treatment. pDNA release, structure, and gene expression are evaluated using standard methods. For in vivo studies, a 1-cm² transducer head is used with aquasonic coupling gel on the tissue surface; a thermocouple is inserted into the tissue site to measure local temperature.

### Monitoring Extent of Bone Regeneration

Tissues are analyzed initially by microCT and then histologically to determine the extent of bone formation. Digital μCT images are taken and reconstructed into a 3-dimensional image with a mesh size of 25 μm×25 μm×25 μm. Scanning may be performed on a GE-EVS high resolution MicroCT System available at the Brigham and Woman core facility, on a per fee basis. Bone volume measures, and calibrated bone mineral density are determined. Quantitative histomorphometric analysis is carried out using standard methods, from plastic embedded sections stained with Goldner's Trichrome stain for osteoid or von Kossa stain for mineralized tissue.

### Statistical Design and Analysis

Sample numbers for all experiments are calculated using InStat Software (Agoura Hills, Calif.), using standard deviations determined in preliminary studies, in order to enable the statistical significance of differences between experimental conditions of greater than 50% to be established. Statistical analysis will be performed using Students t-test (two-tail comparisons), and analyzed using InStat 2.01 software. Differences between conditions are considered significant if p<0.05.

### Spatially Restricted Delivery of Antigen and Tolerogen

Tolerogenic factors like dexamethasone and peptide therapy have been administered to subjects independently (locally or systemically. However, problems have been observed because the dexamethasone has pleiotropic effects throughout the body. Dexamethasone can elicit tolerance in leukocytes that would otherwise alert the body to, or destroy, tumor cells or foreign pathogens. Conversely, peptide delivered to pathogenic cells without a tolerogenic factor can further activate disease.

A challenge with a tolerogenic vaccine formulation is to coordinate the delivery of antigen and tolerogen in space and time to ensure that cells that present antigen are preferentially tolerized and such that those tolerized cells present antigen. If coupling is inadequate, bystander cells presenting third party antigens may become tolerized evoking inappropriate tolerance to antigens from pathogenic microbes or neoplastic cells or in the setting of chronic immune activation antigen may be delivered to activated dendritic cells, worsening disease.

The compositions and methods herein feature linking, e.g., covalent coupling of tolerogens with antigens to coordinate delivery of the antigen and tolerogen. By covalently coupling antigen and tolerogen, the pitfalls that occur when the factors are administered independently are mitigated. Moreover, potency to induce immune tolerance is enhanced when the molecules are delivered in close proximity to one another, e.g., spatially restricted such as covalently coupled.

In accordance with the compositions and methods described herein, tolerogens are delivered in the form of an antigen-tolerogen immunoconjugate. Tolerogens include small molecular weight drugs as well as macromolecules that generate tolerogenic DC that then attenuate T effector responses. Exemplary tolerogens include the glucocorticoids, e.g., dexamethasone. (J. Hu, et al. Immunology 132, 307 (2011); and A. E. Coutinho, et al. Molecular and Cellular Endocrinology 335, 2 (2011)). Dexamethasone is affordable (e.g. does not require recombinant synthesis), has primary alcohol and ketone functional groups for site specific modification, and has been used both in animal models and clinically to prevent, cure, or reduce the severity of allergy/asthma, autoimmune diseases, and transplant rejection. Yet, it has pleiotropic functions in tissues throughout the body and when administered chronically as a bolus, side effects such as osteoporosis, diabetes, Cushing's syndrome, and heart disease can occur. The compositions and methods described herein are of significant clinical importance because only the cells that uptake the antigen uptake the programming factor and vice versa are programmed or reprogrammed, e.g., activated or tolerized. This system in which the antigen and immunomodulatory agent are in close proximity to one another reduces off target effects such that cells that get antigen but not tolerogenic factor become activated and then create immunogenic, not tolerogenic responses.

The results described herein show that the use of a tolerogen-antigen immunoconjugate attenuated side effects normally seen with tolerogen alone, while dampening pathogenic antigen specific immunity. Tolerogen-antigen (e.g., dexamethasone-peptide) conjugates induced tolerance in DC and attenuated T-effector responses in vitro and in vivo. In vivo, the immunoconjugate reduced peak disease severity and clinical score in comparison to the separate tolerogen (e.g., steroid, such as dexamethasone) and antigen (e.g., peptide) components. The conjugate reduced severity of an immune activation disorder compared to separate delivery of tolerogen and antigen.

A strategy for co-delivering antigen and tolerogen is described below, as well as the effects of the immunoconjugate on DC and T cells.

### T Cells

T cells play a critical role in the development and progression of immune activation disorders. However, few methods exist to specifically target the pathogenic T cells.

The compositions described herein, e.g., steroid-peptide immunoconjugates, induced tolerance in dendritic cells while still allowing for antigen presentation. Linking together antigen and tolerogen in space and time led to more potent and specific T cell therapies than previously available. The tolerogenic system described herein elicited tolerogenic DC and allowed for antigen presentation while minimally influencing DC numbers and migration potential.

### Antigens and Immune Activation Disorders

Exemplary antigens suitable for use in the compositions and methods are described above and include lysates of cells associated with an immune activation disorder, peptides, and/or carbohydrate moieties associated with an immune activation disorder. Antigens contain an epitope that initiates or exacerbates immune diseases.

Exemplary immune activation disorders include autoimmune disorders, allergies, asthma, and transplant rejection, and the antigen (e.g., peptide) is associated with an autoimmune disorder, such as multiple sclerosis, type 1 diabetes mellitus, Crohn's disease, rheumatoid arthritis, systemic lupus erythematosus, scleroderma, alopecia areata, antiphospholipid antibody syndrome, autoimmune hepatitis, celiac disease, Graves' disease, Guillain-Barre syndrome, Hashimoto's disease, hemolytic anemia, idiopathic thrombocytopenic purpura, inflammatory bowel disease, ulcerative colitis, inflammatory myopathies, polymyositis, myasthenia gravis, primary biliary cirrhosis, psoriasis, Sjögren's syndrome, vitiligo, gout, atopic dermatitis, acne vulgaris, or autoimmune pancreatitis.

For example, the peptide is associated with multiple sclerosis. Such peptides are, in some cases, derived from proteins such as myelin basic protein, myelin proteolipid protein, myelin-associated oligodendrocyte basic protein, myelin oligodendrocyte glycoprotein, or fragments thereof.

In some embodiments, the peptide is derived from myelin oligodendrocyte glycoprotein (MOG) or myelin basic protein (MBP). In one embodiment, the peptide is derived from MOG. Myelin Oligodendrocyte Glycoprotein (MOG) is a glycoprotein involved in the myelination of nerves in the central nervous system (CNS). MOG is a membrane protein expressed on the surface of oligodendrocytes and in the outermost surface of myelin sheaths. The sequence of the MOG protein is provided in GenBank No. Q61885.1, incorporated herein by reference. In addition to binding to the MHC class II IA^{b} protein, MOG₃₅₋₅₅ (MOG residues 35-55) contains domains that bind to MHC class I molecules and is recognized by CD8+ T cells. (M. L. Ford, et al. European Journal of Immunology 35, 76 (2005)). In some examples, a tolerogen (e.g., dexamethasone)-MOG immunoconjugate manipulates CD4+ T cells and/or CD8+ cells. CD8+ T cells and CD4+T cells have been described to play a role in EAE pathogenesis. See, e.g., R. Aharoni. Expert Review of Clinical Immunology 9, 423 (2013). The amino acid sequence of MOG35-55 is MEVGWYRSPFSRVVHLYRNGK (SEQ ID NO: 8). In some cases, the antigen is directly linked to the tolerogen compound, e.g., a MOG compound or the ovalbumin (siinfekl) compound is directly linked to tolerogen, e.g., Dex. In some cases, a single or few (e.g., 1, 2, 3, 4, 5 or more) amino acid(s), e.g., of the mog compound and the ovalbumin (siinfekl) compound are included. In some embodiments, a single amino acid or a stretch of multiple (e.g., 1, 2, 3, 4, 5, or more) amino acids link an antigen, e.g., a MOG or ovalbumin (siinfekl) compound, to the tolerogenic compound.

Exemplary constructs include dexamethasone-gly-mog, sequences with another small linker located between the antigen and the tolerogen, and two other sequences made without a bridge including dex-siinfekl and dex-TRP-2 (dex-Ser-Val-Tyr-Asp-Phe-Phe-Val-Trp-Leu) (SEQ ID NO: 17).

Myelin basic protein (MBP) is a major component of the myelin sheath of Schwann cells and oligodendrocytes. The nucleotide sequence of an isoform of human MBP is provided by GenBank Accession No. NM_001025081.1, incorporated herein by reference, which encodes the amino acid sequence provided by GenBank Accession No. NP_001020252.1, also incorporated herein by reference.

A peptide suitable for use in the compositions and methods described herein is associated with type I diabetes. For example, the peptide comprises a pancreatic cell-associated peptide or protein. Exemplary pancreatic cell-associated peptides or proteins include insulin, proinsulin, glutamic acid decarboxylase-65 (GAD65), insulinoma-associated protein 2, heat shock protein 60, ZnT8, islet-specific glucose-6-phosphatase catalytic subunit related protein, or fragments thereof.

An antigen (e.g., peptide or lysate) suitable for use in the compositions and methods described herein is associated with allergy or asthma. For example, the antigen comprises an allergen that provokes allergic symptoms, e.g., histamine release or anaphylaxis, in the subject or triggers an asthmatic attack (e.g., acute asthmatic attack). In some embodiments, the allergen comprises (Amb a 1 (ragweed allergen), Der p2 (Dermatophagoides pteronyssinus allergen, the main species of house dust mite and a major inducer of asthma), Betv 1 (major White Birch (Betula verrucosa) pollen antigen), Aln g I from Alnus glutinosa (alder), Api G I from Apium graveolens (celery), Car b I from Carpinus betulus (European hornbeam), Cor a I from Corylus avellana (European hazel), Mal d I from Malus domestica (apple), phospholipase A2 (bee venom), hyaluronidase (bee venom), allergen C (bee venom), Api m 6 (bee venom), Fel d 1 (cat), Fel d 4 (cat), Gal d 1 (egg), ovotransferrin (egg), lysozyme (egg), ovalbumin (egg), casein (milk) and whey proteins (alpha-lactalbumin and beta-lactaglobulin, milk), Ara h 1 through Ara h 8 (peanut), vicilin (tree nut), legumin (tree nut), 2S albumin (tree nut), profilins, heveins, lipid transfer proteins, Cor a 1 (hazelnut), Cor a 1.01 (hazel pollen), Cor a 1.02 (hazel pollen), Cor a 1.03 (hazel pollen), Cor a 1.04 (hazelnut), Bet v 1 (hazelnut), Cor a 2 (hazelnut), glycinin (soybean), Cor a 11 (hazelnut), Cor a 8 (tree nut), rJug r 1 (walnut), rJug r 2 (walnut), Jug r 3 (walnut), Jug r 4 (walnut), Ana o 1 (cashew nut), Ana o 2 (cashew nut), Cas s 5 (chestnut), Cas s 8 (chestnut), Ber e 1 (Brazil nut), Mal d 3 (apple), Pru p 3 (peach) or gluten. See, e.g., Roux et al. Int Arch Allergy Immunol 2003; 131:234-244, incorporated herein by reference.

Allergic conditions include, e.g., latex allergy; allergy to ragweed, grass, tree pollen, and house dust mite; food allergy such as allergies to milk, eggs, peanuts, tree nuts (e.g., walnuts, almonds, cashews, pistachios, pecans), wheat, soy, fish, and shellfish; hay fever; as well as allergies to companion animals, insects, e.g., bee venom/bee sting or mosquito sting.

In some embodiments, the antigen (e.g., peptide or lysate) is associated with transplant rejection.

Exemplary antigens, e.g., alloantigens, associated with transplant rejection, include a major histocompatibility complex (MHC) molecule (e.g., MHC class I or II antigen), HLA class I molecules (e.g., HLA-G), a minor H antigen (which is a peptide derived from a polymorphic protein that is presented by the MHC molecules of the transplanted cells/tissues), endothelial receptors, adhesion molecules, intermediate filaments, and the MICA/B and the KIR receptor complex, or fragments thereof. In one example, a minor H antigen includes HB-1, which is a B-cell lineage marker expressed by acute lymphoblastic leukemia cells.

### Tolerogens

Tolerogens suitable for use in the compositions and methods described herein include dexamethasone, vitamin D, retinoic acid, thymic stromal lymphopoietin, rapamycin, aspirin, transforming growth factor beta, interleukin-10, vasoactive intestinal peptide, and/or vascular endothelial growth factor.

In some embodiments, a tolerogen suitable for use in the compositions and methods described herein minimally interferes with dendritic cell migration. In some cases, the tolerogen facilitates dendritic cell migration, e.g., toward an administered immunoconjugate or toward a lymph node.

### Effects of Tolerogens

Decreased surface expression of CD80, CD86, and MHC II demonstrated the formation of tolerogenic DC. T cell responses were obtained in a mixed leukocyte reaction (MLR). For example, decreased expression of inflammatory markers such as IL-12, IL-6, TNF-alpha, and IFNs with concomitant enhancement of tolerogenic factors such as IL-10, TGF-beta, and IDO, demonstrated formation of tolerogenic DC. Formation of tolerogenic DC can also be demonstrated by other tests, such as cytokine ELISAs for IL-10, IL-12, IFNs, TNF, and/or IL-6. For example, the presence of one or more of the cytokines and a tolerogenic T cell response in a MLR confirm tolerogenic DC. A tolerogen, e.g., Dexamethasone, inhibited LPS based activation of dendritic cells, which led to the attenuation of T cell proliferation. There was reduced T cell activity in the presence of the tolerogen, e.g., steroid. The tolerogen, e.g., dexamethasone, inhibited T cell proliferation in a dose-responsive manner, demonstrating the formation of tolerogenic DC.

To induce tolerance, the compositions described herein enrich dendritic cell numbers locally and deliver dendritic cells to the lymph node. For example, the compositions do not elicit adverse side effects, e.g., do not inhibit the accumulation of dendritic cells or their delivery to the lymph node. For example, the compositions do not alter migration or induce cell death of dendritic cells. Rapamycin is a potent tolerogenic (and immunogenic under certain conditions) factor that inhibits leukocyte trafficking, e.g., trafficking to GM-CSF. (J. N. Defrancischi, et al. British Journal of Pharmacology 110, 1381 (1993); and J. Gomez-Cambronero. FEBS Letters 550, 94 (2003)). In some cases, the compositions described herein do not include rapamycin as a tolerogen. For example, induction of cell death is not desired because, in addition to decreasing the effective number of DC that could be programmed, changes in programmed cell death could worsen disease or lead to autoimmunity. (M. Chen, et al. Immunol. Rev. 236, 11 (2010)). If the DC are correctly programmed, more DCs lead to a more potent tolerogenic vaccine. Also, changes in programmed cell death potentiate immunity, e.g., if immunogenic DC or T cells persist, inflammation could worsen.

Tolerogens, e.g., dexamethasone, had nominal impact on dendritic cell numbers and minimally influenced dendritic cell migration. Only suprapharmacologic doses, e.g., of 10⁻⁶ M tolerogen (e.g., dexamethasone) caused changes in cell numbers. In some examples, a ceiling for how high the concentration of a tolerogen can be at a vaccine site before causing deleterious effects is based on the highest dose that does not cause significant changes in dendritic cell numbers. For example, in accordance with the compositions and methods described herein, the concentration of a tolerogen at a vaccine site is less than 10⁻⁶ M, e.g., 9×10⁻⁵ M, 5×10⁻⁵ M, 2.5×10⁻⁵ M, 1×10⁻⁵ M, or less.

DC migrated toward the tolerogen, e.g., dexamethasone, as did tolerogen-treated DC to CCL19. For migration to CCL20, the result was significant at the 0.05 level, demonstrating that migration to the vaccine site and lymph node were not hindered with tolerogen (e.g., dexamethasone), and potentially was augmented.

In some embodiments, a tolerogenic immunoconjugate described herein inhibits dendritic cell maturation, is presented to T cells, and/or inhibits T cell proliferation.

### Tolerogenic Immunoconjugates

The compositions and methods described herein localize antigen and tolerogen in space and time, thereby enhancing vaccine potency and reducing side effects. Covalent conjugation with covalent bonds or a linker whereby both molecules (e.g., antigen and tolerogen) are delivered to the same cell with neither molecule delivered alone, limits the likelihood of tolerogen inappropriately inducing tolerance or antigen being presented in an inflammatory context. Linkers include peptide linkers, e.g., varying from 1 to 10 or more amino acids, click chemistry, variety of others known in the art. Other examples include carbamate, amide, ester bond or carbodiimide linkage (a few atoms to up to as many as desirable). Covalent coupling increases the likelihood that a cell that uptakes the antigen will also become tolerogenic. Covalent coupling limits off target effects of delivering antigen to activated cells and tolerogen to other cells carrying third party antigens. For example, the results described herein show that a tolerogen, e.g., dexamethasone, was incorporated into a peptide immunoconjugate, the conjugation was performed, e.g., in a semi-automated manner, and the approach worked with a variety of peptides, e.g., MOG, TRP2, and ovalbumin (e.g., SIINFEKL) peptides.

In some embodiments, a composition described herein includes a tolerogenic immunoconjugate as well as an immunomodulator drug, e.g., Glatiramer acetate (also called Copaxone®). For example, the immunomodulatro drug, e.g., Copaxone®), is covalently linked to dexamethasone or another tolerogen. Glatiramer acetate is a mixture of synthetic peptides that mimic myelin basic protein (MBP). Glatiramer acetate is composed of the amino acids, glutamic acid, lysine, alanine, and tyrosine. The amino acids are assembled in random order into polypeptides having 40-100 amino acids. In some examples, the coupling strategy is used to link an antigen to an extant tolerogenic molecule that targets either DC or T cells. For example, DC may shuttle the antigen-tolerogen to the T cells in the draining lymph node and thereby target them. Any immunosuppressive, e.g., steroids, rapamycin, methotrexate, tacro, or cyclosporin, is suitable as a tolerogen. In terms of allergy therapy, an exemplary suitable tolerogen includes omalizumab. For MS therapy, there are number of agents that have orthogonal modes of action that likely exhibit synergy when used in the compositions and methods described herein. Such agents include the following compounds: Aubagio (teriflunomide); Avonex (interferon beta-1a); Betaseron (interferon beta-1b); Copaxone (glatiramer acetate); Extavia (interferon beta-1b); Gilenya (fingolimod); Lemtrada (alemtuzumab); Novantrone (mitoxantrone); Plegridy (peginterferon beta-1a); Rebif (interferon beta-1a); Tecfidera (dimethyl fumarate); and/or Tysabri (natalizumab).

### Dexamethasone-Antigen Conjugates

A dexamethasone-antigen conjugate, e.g., dexamethasone-SIINFEKL (SEQ ID NO: 9), inhibited the increase in surface expression of MHC II, CD80, and CD86 following challenge, e.g., with the toll-like receptor ligand LPS. The potency of dexamethasone and the peptide conjugate were nearly equivalent. The anti-inflammatory properties of dexamethasone in terms of the surface expression of MHC II, CD80, and CD86 were maintained following functionalization with a peptide, e.g., at the 21^{st} carbon of dexamethasone. Other dexamethasone-peptide conjugates are provided herein.

The immunoconjugate, e.g., dexamethasone-peptide conjugate elicited a tolerogenic phenotype in DC. For example, antibody binding to the surface of DCs pulsed with a dexamethasone peptide conjugate, e.g., dexamethasone-SIINFEKL (SEQ ID NO: 9), was reduced compared to peptide (e.g., SIINFEKL (SEQ ID NO: 9)) alone, reducing the likelihood of T cell expansion upon TCR binding. The amount of staining present in the peptide (e.g., SIINFEKL (SEQ ID NO: 9)) alone or peptide (e.g., SIINFEKL (SEQ ID NO: 9)) and dexamethasone-peptide (e.g., Dex-SIINFEKL (SEQ ID NO: 9)) groups was indistinguishable. The resulting DCs had a tolerogenic phenotype.

The compositions described herein, e.g., tolerogenic immunoconjugates, induce tolerogenic DC and/or induce a tolerogenic phenotype upon exposure to DC. In some cases, the compositions described herein, e.g., tolerogenic immunoconjugates, are displayed by DC. The compositions do not inhibit DC trafficking and minimally affect the number of DC. For example, the amount of tolerogen in the composition is such that minimal adverse effects (e.g., change in DC number, e.g., reduction) are elicited. For example, the amount of tolerogen in the composition is 0.05-500 mg (e.g., 0.1-500 mg, 0.1-250 mg, 0.1-100 mg, 1-500 mg, 1-250 mg, 1-100 mg, 10-500 mg, 10-250 mg, 10-100 mg, or 100-500 mg).

### Preparation of Conjugate

The compositions described herein include an antigen covalently linked to a tolerogen. “Covalently linked” molecules include molecules linked by one covalent bond, or linked by more than one covalent bond (e.g., 2, 3, 4, 5, 6, 7, 8, 9, 10, 11, 12, 13, 14, 15, 16, 17, 18, 19, 20, or more), e.g., linked by a linker or spacer. In some cases, the antigen and tolerogen are covalently attached by a bond, e.g., a carbamate, amide, or ester bond. In some cases, the antigen and tolerogen are covalently attached by a linker or spacer. In some cases, the antigen and tolerogen are connected by a carbodiimide linkage. An exemplary linker includes a dex-hemisuccinate coupled to the free amine on the solid phase peptide chain forming an amide bond through the 21^{st} carbon of dexamethasone. Another example of a linker is Dex-NHS directly coupled through the 21^{st} carbon of dexamethasone to the free amine on the phase chain. An additional example of a linker is Dex carried by a cyclodextrin via van der waals interactions. The 21^{st} carbon of dexamethasone is the carbon of the ketone that is bound to a hydroxyl. For example, the tolerogen is linked to the N-terminus of a peptide antigen, e.g., via solid phase chemistry (e.g., FMOC solid phase chemistry). In other examples, the tolerogen is linked to the C-terminus of a peptide antigen, e.g., via solution phase chemistry.

Different immune cell types are targeted depending on linker/linkage half-life. For example, if the hydrolysis time constant of a tolerogen-antigen conjugate is close to the time constant of conjugate uptake by DC, then DC would be targeted with tolerogen alone. However, if the kinetics of tolerogen-antigen cleavage match the time constant of DC trafficking to the lymph node, then the tolerogen may be released from DC carriers to proximal T cells (e.g., antigen specific T cells). In some cases, antigen presenting cell (APC) specific cleavage sites such as the Val-Val-Arg sequence are used to more selectively target enzymatic cleavage in DC. H. A. Chapman. Current Opinion in Immunology 18, 78 (2006). For example, the linkage/linker is designed with a certain cleavage rate such that both DC and antigen specific T cells are targeted with the tolerogen by matching hydrolysis rates with immunoconjugate trafficking. In some examples, the covalent linking strategy (e.g., coupling chemistry), e.g., with different half-lives and/or enzymatic cleavage sequences, are specifically designed to target specific leukocyte populations in the periphery and/or the lymph node. For example, a MMP (e.g., MMP-9 or MMP-2) cleavage sequence includes valine-valine-arginine.

In some examples, one or more, e.g., a plurality of, (e.g., 2, 3, 4, 5, 6, 7, 8, 9, 10, or more) antigens are mixed together, e.g., coupled to one or more tolerogens (e.g., 1, 2, 3, 4, 5, 6, 7, 8, 9, 10, or more tolerogens), e.g., to form a tolerogenic cocktail, to provide broader antigenic coverage than with one antigen alone. In some cases, a composition containing more than one antigen and/or tolerogen (e.g., linked and/or mixed together) inhibits immunity when multiple pathogenic T cell responses exist. For example, one or more myelin antigens, or myelin antigen peptides coupled to a tolerogen described herein are useful for treating MS. See, e.g., (A. Lutterotti et al. Science Translational Medicine 5, (2013)). In another embodiment, an immunoconjugate is coupled to a protein.

### Production of Immunoconjugates

Dexamethasone has been coupled to a variety of biomolecules, including synthetic polymers, proteins, nanofibers, and polycations. (M. D. Howard et al. Pharmaceutical Research 28, 2435 (2011). C. D. Jones, et al. Steroids 23, 323 (1974, 1974); and R. Bucki et al. Antimicrobial Agents and Chemotherapy 54, 2525 (2010); and M. J. Webber, et al. Biomaterials 33, 6823 (2012)). (C6 and C3 with the double bond to oxygen and C21 bound to the primary hydroxyl have been derivatized as exemplified in (M. D. Howard et al. Pharmaceutical Research 28, 2435 (2011); and C. D. Jones, et al. Steroids 23, 323 (1974, 1974), respectively)). Amino acids and proteins are coupled using a variety of solution phase techniques through the primary hydroxyl or the conjugated oxygen (X. M. Liu et al., Biomacromolecules 11, 2621 (2010); and H. Kim et al. Journal of Cellular Biochemistry 110, 743 (2010)). For example, a tolerogen and an antigen are coupled by a solution phase technique using standard methods known in the art. In other cases, a solid phase technique is used for coupling. For example, a solid phase technique in some cases reduces time and facilitates automation of the synthesis and purification of the final product. See, e.g., (K. C. Koehler, Biomaterials 34, 4150 (2013) and US 20090061014 A1, incorporated herein by reference). The solid phase synthesis technique is also applicable to the synthesis of other steroid-peptide conjugates, biotinylated compounds, or fluorescently labeled peptides.

Hydrolysis of an immunoconjugate affects drug delivery and overall bioactivity. For example, there is a short window for DC to encounter antigen bound to tolerogen prior to immunoconjugate scission—this affects drug efficacy. The half-life of a tolerogen-antigen linkage/linker is modulated by using different linkages/linkers. For example, the half-life increased by replacing an ester linkage with a carbamate group. K. C. Koehler, Biomaterials 34, 4150 (2013). In some examples, a carbamate tolerogen (e.g., carbamate dexamethasone) moiety is added to an antigen, e.g., peptide, by solid-phase peptide synthesis. K. C. Koehler, Biomaterials 34, 4150 (2013). In some cases, an immunoconjugate with a longer half-life allows for antigen specific T cell targeting with tolerogen (e.g., dexamethasone) in the lymph node, whereby the DC function as carriers delivering the tolerogen (e.g., dexamethasone) to the lymph node resident T cells.

In some cases, the rate of bond (e.g., ester bond) hydrolysis is about the same as or lower than the rate of diffusion of the conjugated molecule to a dendritic cell. For example, using the Hydrowin v 2.00™ (E. P. Agency. (2012), vol. 2013) software program from the Environmental Protection Agency the predicted rate of aqueous hydrolysis for a compound similar to a dexamethasone peptide conjugate was 0.7 L/(mol-s) at 25° C. at pH 8 giving a half-life of 10.9 days. At a pH of 7, the half-life extended to 109 days (the compound tolerated a 95% TFA cleavage cocktail at RT). Experimentally, in PBS the time constant for enzyme hydrolysis of dexamethasone hemisuccinate or a similar dexamethasone conjugate bound to a poly (ethylene glycol) gel was found to be ½ to 1 day. (C. R. Nuttelman. Journal of Biomedical Materials Research Part A 76A, 183 (2006). In some cases, the immunoconjugate compounds described herein have a similar degradation rate as described above. At early time points (e.g., within 24 hours, e.g., within 20, 18, 16, 14, 12, 10, 8, 7, 6, 5, 4, 3, 2, or 1 hours after administration of the conjugate), for example, antigen and tolerogen are available for co-delivery without depending upon biomaterial release platforms. In vivo, hydrolysis can also occur enzymatically via enzymes which may reduce the time constant. This difficulty is overcome in some cases using drug delivery strategies to shield the prodrug. (J. Rautio et al. Nature Reviews Drug Discovery 7, 255 (2008); and B. M. Liederer. Journal of Pharmaceutical Sciences 95, 1177 (2006)).

### Delivery Device

In some examples, an immunoconjugate described herein is not provided in a delivery device, e.g., it is delivered via fluid phase injection (bolus administration) in the absence of a delivery vehicle (e.g., microchip or polymeric matrix delivery vehicle). In other embodiments, the immunoconjugate is provided in or incorporated into or onto a delivery device, e.g., a polymeric matrix or microchip. For example, a suitable microchip is described, e.g., in Santini et al. Nature 397(1999):335-38, incorporated herein by reference.

Material systems, e.g., delivery scaffolds, can be beneficial in terms of their ability to enhance the distribution of the antigen-tolerogen to certain sites in the body, to recruit cells to a local controlled environment, and to control the delivery of the component in space and time. For example, the material system facilitates the delivery of the conjugate to certain tissues, e.g., peripheral locations, or the draining lymph nodes (e.g., places with the most tolerogenic DC). Alternatively, the disease site is targeted directly using effects such as enhanced permeability and retention (EPR). Examples of targeting strategies include injectable formulations, nanoparticles, or antibody carriers. In other examples, material systems provide a method of controlling the delivery of substances spatially and temporally. For example, the material system provides a localized environment distinct from the disease site that recruits specific cell populations and programs them in a continuous manufacturing manner. In some cases, the material system is capable of evoking more potent responses by first recruiting a critical number of DC and then delivering the immunoconjugate to these cells. In some examples, the material system is designed such that only DCs are targeted (e.g., by coupling the compound to materials, e.g., gels, with DC-specific linkages). In other examples, the delivery is responsive to certain environmental cues (e.g., status after being stung by a bee, or a multiple sclerosis disease flare).

In some cases, the device comprises a microchip or a polymer. For example, the polymer comprises alginate, poly(ethylene glycol), hyaluronic acid, collagen, gelatin, poly (vinyl alcohol), fibrin, poly (glutamic acid), peptide amphiphiles, silk, fibronectin, chitin, poly(methyl methacrylate), poly(ethylene terephthalate), poly(dimethylsiloxane), poly(tetrafluoroethylene), polyethylene, polyurethane, poly(glycolic acid), poly(lactic acid), poly(caprolactone), poly(lactide-co-glycolide), polydioxanone, polyglyconate, BAK; poly(ortho ester I), poly(ortho ester) II, poly(ortho ester) III, poly(ortho ester) IV, polypropylene fumarate, poly[(carboxy phenoxy)propane-sebacic acid], poly[pyromellitylimidoalanine-co-1,6-bis(p-carboxy phenoxy)hexane], polyphosphazene, starch, cellulose, albumin, polyhydroxyalkanoates, Poly(lactide), or poly(glycolide).

Exemplary delivery devices, components of delivery devices, and methods of making delivery devices are described in U.S. Pat. No. 8,067,237; U.S. Patent Application Publication No. 2012/0100182; U.S. Patent Application Publication No. 2013/0202707; U.S. Patent Application Publication No. 2013/0177536; U.S. Pat. No. 8,728,456; U.S. Patent Application Publication No. 2014/0079752; U.S. Patent Application Publication No. 2012/0122218; U.S. Patent Application Publication No. 2013/0302396; U.S. Patent Application Publication No. 2014/0112990; U.S. Patent Application Publication No. 2014/0227327; and U.S. Patent Application Publication No. 2014/0178964, all of which are incorporated by reference in their entireties.

In some examples, the polymer comprises a capsular polysaccharide A from B. fragilis. In some cases, the polysaccharide A is used in a tolerogenic platform, such as a macroporous cryogel. For example, the polysaccharide is both a tolerogen as well as a polymer for the scaffold.

The polymer is neutral, hydrophobic, or hydrophilic. Examples of hydrophobic polymers include a polyanhydride and a poly (ortho ester), PLGA, and polycaprolactone. Examples of hydrophilic polymers include alginate, PEG, methacrylates (polyacrylamides), collagen, fibrin, hyaluronan, and poly vinyl alcohol.

In some examples, the delivery device contains pores, e.g., macropores, micropores, and/or nanopores. For example, the diameter of nanopores are less than about 10 nm; micropore are in the range of about 100 μm-20 μm in diameter; and, macropores are greater than about 20 μm (preferably greater than about 100 μm and even more preferably greater than about 400 μm, e.g., greater than 600 μm or greater than 800 μm). In some examples, pore size is less than about 10 nm, in the range of about 100 nm-20 μm in diameter, or greater than about 20 μm, e.g., up to and including 1000 μm. The size of the pores allows the migration into and subsequent exit of cells such as DCs from the device. In one example, the scaffold is macroporous with open, interconnected pores of about 30-600 μm in diameter, e.g., 30-200, 100-200, 200-400, or 400-600 μm. In some cases, the size of the pores and the interconnected architecture allows the cells to enter, traverse within the volume of the device via the interconnected pores, and then leave the device via the pores to go to locations in the body outside of the device. For DCs, a preferred pore size range is 30 to 600 μm. If recruiting factors are included this range may change as the delivery kinetics of the factors change as a function of the pores and the mechanical strength changes. Nanoporous, e.g., pores with a diameter scale of nanometers (typically between 0.1 and 100 nanometers) materials are also useful.

In some examples, the immunoconjugate is hydrolyzed following incorporation into a device, e.g., a poly(d,l-lactide-co-glycolide) (PLG) scaffold, e.g., within 12 months, e.g., within 12, 11, 10, 9, 8, 7, 6, 5, 4, 3, 2, 1 month, 5, 4, 3, 2, 1 week, 7, 6, 5, 4, 3, 2, 1 day, 24, 12, 6, 4, 2, or 1 hour, e.g., at body temperature, e.g., around 37° C. In some cases, a polymeric biomaterial delivery device is used that has hydrophobic matrix with low water diffusivity. For example, polyanhydrides and poly (ortho esters) are examples of a relatively more hydrophobic matrix with low water diffusivity. For example, porous (e.g., macroporous) biomaterials are used for drug delivery to enrich DC at the site of immunoconjugate exposure and enhance potency of the immunoconjugate in eliciting a tolerogenic response.

Hydrolysis rates within a delivery device of a tolerogen-antigen linkage/linker are optimized for the desired tolerogenic effects. For example, the linkage/linker is modulated by changing the linkage chemistry. In other examples, hydrophobic carriers, such as the polyanhydride or poly (ortho esters) polymer families (e.g., containing a low concentration of water/nucleophiles and/or a low rate of diffusion of water/nucleophiles) are used as delivery devices. In other situations, drug delivery chips are used to delivery immunoconjugates. (J. T. Santini, et al. Nature 397, 335 (1999)).

The delivery device optionaly includes a DC recruitment composition, such as GM-CSF, in addition to an immunoconjugate. For example, the recruitment composition (e.g., GM-CSF) accumulates DC at the administration site. GM-CSF can have either activating or tolerizing properties depending upon its dose, duration, and administration site. See, e.g., (J. L. McQualter et al. Journal of Experimental Medicine 194, 873 (2001); and M. El-Behi et al. Nature Immunology 12, 568 (2011)).

The dose and duration of recruitment composition (e.g., GM-CSF) delivery to DCs is optimized to elicit the desired tolerogenic effects. Other DC enrichment compositions are suitable for use in the delivery devices described herein. For example, DC recruitment compositions include but are not limited to granulocyte-macrophage colony stimulating factor (GM-CSF), FMS-like tyrosine kinase 3 ligand, N-formyl peptides, fractalkine, monocyte chemotactic protein-1, and macrophage inflammatory protein-3 (MIP-3α). Flt3L has been described to enhance local DC numbers for macroporous PLG scaffolds, and Flt3/Flt3L has been described to expand peripheral DC populations and used to inhibit autoimmunity. See, e.g., O. A. Ali, et al. Advanced Functional Materials 23, 4621 (2013).

Endogenous GM-CSF polypeptides may be isolated from healthy human tissue. Synthetic GM-CSF polypeptides may be synthesized in vivo following transfection or transformation of template DNA into a host organism or cell, e.g. a mammal or cultured human cell line. Alternatively, synthetic GM-CSF polypeptides are synthesized in vitro by polymerase chain reaction (PCR) or other art-recognized methods Sambrook, J., Fritsch, E. F., and Maniatis, T., Molecular Cloning: A Laboratory Manual. Cold Spring Harbor Laboratory Press, NY, Vol. 1, 2, 3 (1989), herein incorporated by reference).

GM-CSF polypeptides may be modified to increase protein stability in vivo. In some embodiments, GM-CSF polypeptides are engineered to be more or less immunogenic. Endogenous mature human GM-CSF polypeptides are glycosylated, reportedly, at amino acid residues 23 (leucine), 27 (asparagine), and 39 (glutamic acid) (see U.S. Pat. No. 5,073,627, the entire content of which is incorporated herein by reference). GM-CSF polypeptides of the present invention are modified at one or more of these amino acid residues with respect to glycosylation state. In some embodiments, the GM-CSF polypeptides are recombinant. In various embodiments, GM-CSF polypeptides are humanized derivatives of mammalian GM-CSF polypeptides. Exemplary mammalian species from which GM-CSF polypeptides are derived include, but are not limited to, mouse, rat, hamster, guinea pig, ferret, cat, dog, monkey, or primate. In an embodiment, GM-CSF is a recombinant human protein (PeproTech, Catalog #300-03). In certain embodiments, GM-CSF is a recombinant murine (mouse) protein (PeproTech, Catalog #315-03). GM-CSF may also be a humanized derivative of a recombinant mouse protein.

Human Recombinant GM-CSF (PeproTech, Catalog #300-03) is encoded by the following polypeptide sequence (SEQ ID NO: 10):

(SEQ ID NO: 10)
MAPARSPSPS TQPWEHVNAI QEARRLLNLS RDTAAEMNET
VEVISEMFDL QEPTCLQTRL ELYKQGLRGS LTKLKGPLTM
MASHYKQHCP PTPETSCATQ IITFESFKEN LKDFLLVIPF
DCWEPVQE

Murine Recombinant GM-CSF (PeproTech, Catalog #315-03) is encoded by the following polypeptide sequence (SEQ ID NO: 11):

(SEQ ID NO: 11)
MAPTRSPITV TRPWKHVEAI KEALNLLDDM PVTLNEEVEV
VSNEFSFKKL TCVQTRLKIF EQGLRGNFTK LKGALNMTAS
YYQTYCPPTP ETDCETQVTT YADFIDSLKT FLTDIPFECK
KPVQK

Human Endogenous GM-CSF is encoded by the following mRNA sequence (NCBI Accession No. NM_000758, hereby incorporated by reference; SEQ ID NO: 12):

| (SEQ ID NO: 12) | |
|---|---|
| 1 | acacagagag aaaggctaaa gttctctgga ggatgtggct gcagagcctg ctgctcttgg |
| 61 | gcactgtggc ctgcagcatc tctgcacccg cccgctcgcc cagccccagc acgcagccct |
| 121 | gggagcatgt gaatgccatc caggaggccc ggcgtctcct gaacctgagt agagacactg |
| 181 | ctgctgagat gaatgaaaca gtagaagtca tctcagaaat gtttgacctc caggagccga |
| 241 | cctgcctaca gacccgcctg gagctgtaca agcagggcct gcggggcagc ctcaccaagc |
| 301 | tcaagggccc cttgaccatg atggccagcc actacaagca gcactgccct ccaaccccgg |
| 361 | aaacttcctg tgcaacccag attatcacct ttgaaagttt caaagagaac ctgaaggact |
| 421 | ttctgcttgt catccccttt gactgctggg agccagtcca ggagtgagac cggccagatg |
| 481 | aggctggcca agccggggag ctgctctctc atgaaacaag agctagaaac tcaggatggt |
| 541 | catcttggag ggaccaaggg gtgggccaca gccatggtgg gagtggcctg gacctgccct |
| 601 | gggccacact gaccctgata caggcatggc agaagaatgg gaatatttta tactgacaga |
| 661 | aatcagtaat atttatatat ttatattttt aaaatattta tttatttatt tatttaagtt |
| 721 | catattccat atttattcaa gatgttttac cgtaataatt attattaaaa atatgcttct |
| 781 | a |

Human Endogenous GM-CSF is encoded by the following amino acid sequence (NCBI Accession No. NP_000749.2, hereby incorporated by reference; SEQ ID NO: 13):

(SEQ ID NO: 13)
MWLQSLLLLGTVACSISAPARSPSPSTQPWEHVNAIQEARRLLNLSRDTA
AEMNETVEVISEMFDLQEPTCLQTRLELYKQGLRGSLTKLKGPLTMMASH
YKQHCPPTPETSCATQIITFESFKENLKDFLLVIPFDCWEPVQE

Residues 1-17, i.e., MWLQSLLLLGTVACSIS (SEQ ID NO: 30), of SEQ ID NO: 13 above correspond to the signal peptide.

An exemplary amino acid sequence of human Flt3 is provided below (GenBank Accession No.: P49771.1 (GI:1706818), incorporated herein by reference; SEQ ID NO: 14):

| (SEQ ID NO: 14) | |
|---|---|
| 1 | mtvlapawsp ttylllllll ssglsgtqdc sfqhspissd favkirelsd yllqdypvtv |
| 61 | asnlqdeelc gglwrlvlaq rwmerlktva gskmqgller vnteihfvtk cafqpppscl |
| 121 | rfvqtnisrl lqetseqlva lkpwitrqnf srclelqcqp dsstlpppws prpleatapt |
| 181 | apqpplllll llpvglllla aawclhwqrt rrrtprpgeq vppvpspqdl llveh |

In some examples, a mesenchymal stem cell (MSC) recruitment composition is included in the composition/device. MSC have been described to facilitate tolerance induction. (A. Bartholomew et al. Experimental Hematology 30, 42 (2002); and M. Di Nicola et al., Blood 99, 3838 (2002)). Examples of MSC recruitment factors include stromal-derived factor 1, hepatocyte growth factor, and Sialyl Lewis(x) agonists.

The delivery device, e.g., polymeric scaffold, e.g., macroporous polymer scaffold, delivers DC recruitment composition(s) in a controlled spatio-temporal manner. For example, alginate cryogels (e.g., macroporous) that are immunologically inert are used in the delivery device. In other examples, PLG is used in the delivery vehicle. Other suitable materials include polyanhydride and poly (ortho ester) surface eroding materials. For example, such materials avoid the burst phase of factor release and instead deliver factors constantly for an arbitrary time frame, e.g., at least 1 hour (e.g., at least 1, 2, 3, 4, 5, 6, 12, 24 hours, 1, 2, 3, 4, 5, 6, 7 days, 1, 2, 3, 4, 5 weeks, 1, 2, 3, 4, 5, 6, 12, 24, 48 months, or greater). In some cases, delivery device materials release a dose of recruitment composition (e.g., GM-CSF) constantly. For example, delivery parameters that enrich for large numbers of DC and induce tolerance are used. See, e.g., (P. Serafini et al., Cancer Research 64, 6337 (2004); and 271. S. A. Rosenberg et al., Journal of Immunology 163, 1690 (1999); and S. J. Simmons et al., Prostate 39, 291 (1999); and M. von Mehren et al. Clinical Cancer Research 7, 1181 (2001)).

In some cases, the delivery device avoids an immunogenic burst phase. For example, the delivery device contains a material where D_{water}>D_{scission} (the diffusion constant in water is greater than the scission constant, meaning the rate limiting step is the scission).

In some embodiments, the delivery vehicle comprises mesoporous silica (MPS). With respect to promoting an immune response, delivery of an immunoconjugate comprising an adjuvant conjugated to an antigen (e.g., a peptide antigen conjugated to a carrier protein), from a MPS vaccine scaffold increases the immunogenicity and humoral responses against the antigen or peptide as compared to delivering the antigen and adjuvant as separate entities. The peptide antigen may comprise, e.g., a B cell epitope. In some embodiments, antibody generation against the peptide requires a CD4 epitope (CD 4 T cell help), which is present on the carrier protein and/or adjuvant. In certain embodiments, the carrier protein is an antigen with a CD4 epitope that, when conjugated to an antigen of interest (e.g., an antigen whose peptides are poorly presented by immune cells when administered alone), increases presentation of a peptide from the antigen or interest or activation of T cells by a peptide of the antigen of interest. In some embodiments, a peptide that might not otherwaise be presented (e.g., exposed or displayed) on the surface of an immune cell is presented when the peptide is conjugated to a carrier protein. Thus, in certain embodiments, an antigen of interest may benefit from or become part of the CD4 response of another antigen that the antigen of interest is conjugated to.

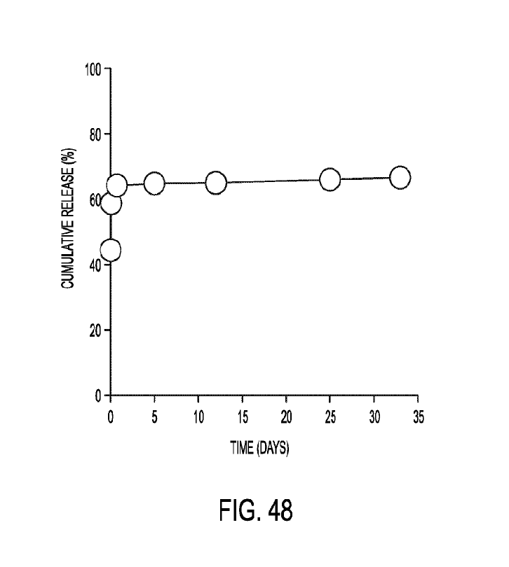

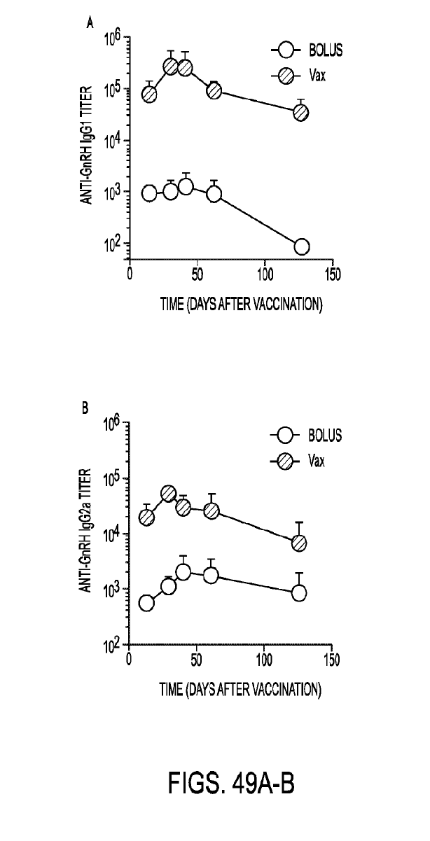

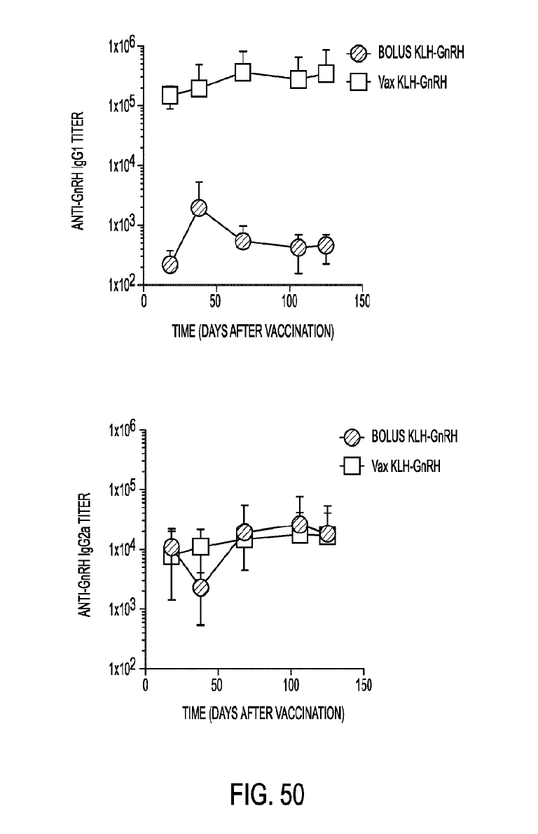

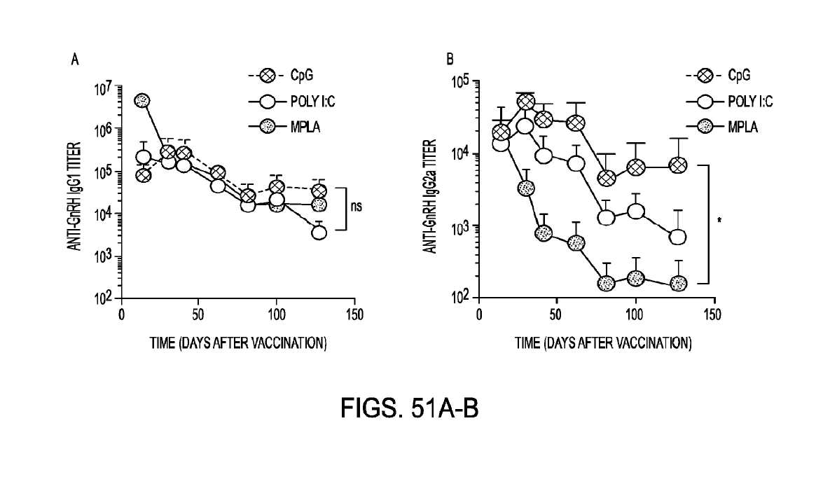

In some embodiments, a peptide containing a cysteine is conjugated to a carrier protein through maleimide (sulfhydryl-sulfhydryl) linkers. Success of conjugation and enhanced humoral response has been shown using a small Gonadotropin-releasing hormone peptide (GnRH). See FIGS. 47-51. This enhanced effect was also seen using both ovalbumin (OVA) and Keyhole limpet hemocyanin (KLH). See FIG. 50.

In various embodiments, if a compatible functional groups (e.g., for disulfide, click, or other linking) are present on a delivery device scaffold composition and a compound (e.g., an antigen or an immunoconjugate comprising an antigen), then the compound and the delivery device scaffold may be directly conjugated (e.g., without a linker or spacer) via a covalent bond. One non-limiting example is a disulfide bond between two cysteines. If the right/compatible functional groups are not present on the delivery device scaffold composition and a compound, then a linker or spacer may be used to conjugate the compound to the scaffold.

Various non-limiting examples of delivery device scaffold compositions are disclosed herein. In some embodiments, the scaffold composition comprises PLGA, a cryogel, MPS, and/or a pore-forming gel (e.g., a gel that forms macropores).

In some embodiments, the scaffold composition comprises MPS. MPS may itself be use as an immunomodulatory agent, e.g., an aduvant. Thus, aspects of the present subject matter provide an immunoconjugate comprising an antigen that is conjugated to MPS. In some embodiments, the MPS is an MPS particle and/or rod. For example, the MPS particle or rod may have a diameter or length of about 1, 2, 3, 4, 5, 6, 7, 8, 9, 10, 25, 50, 100, 200, 300, 400, 500, 600, 1000, 1500, 2000 nm or more. In some embodiments, the MPS is in the form of a rod that has a length of at least about 5, 10, 15, 25, 50, 100, 150, 200, 300, 400, 500, or 5-500 μm. Non-limiting examples of MPS rods are described in U.S. Patent Application Publication No. 2015/0072009.

Various implementations of the present subject matter relate to the conjugation of a peptide directly to a MPS vaccine scaffold to increase the immunogenicity of the peptide and/or to prolong the local presentation of the peptide in vivo. MPS structural material has proinflammatory, e.g., adjuvant properties. Therefore, direct conjugation of an antigen to MPS enhances the efficiency and duration of antigen presentation by APCs.

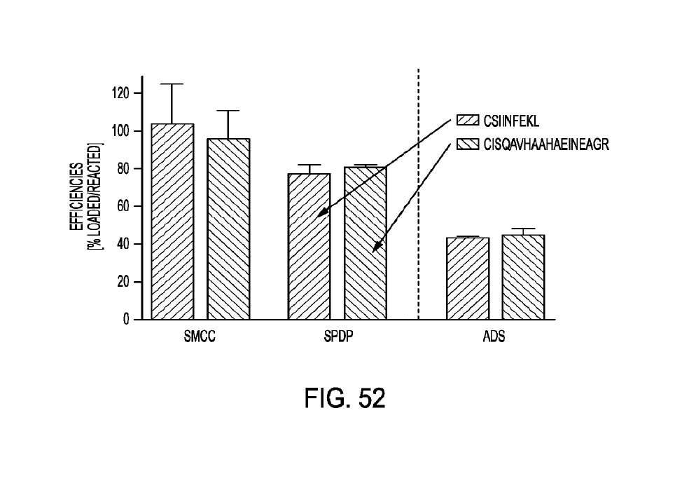

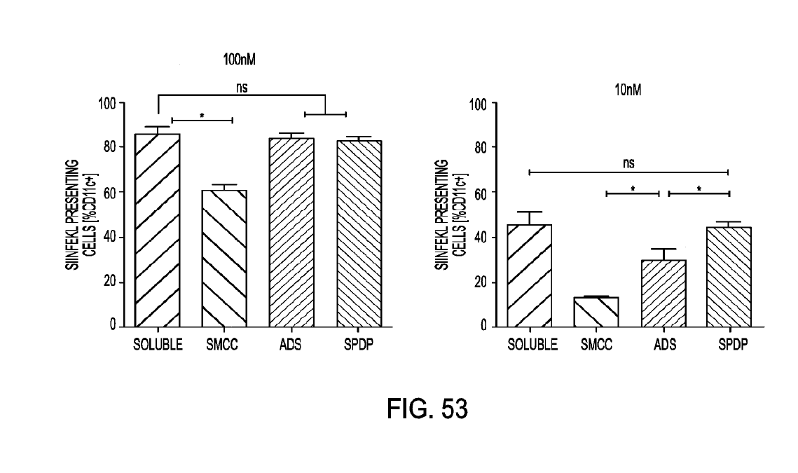

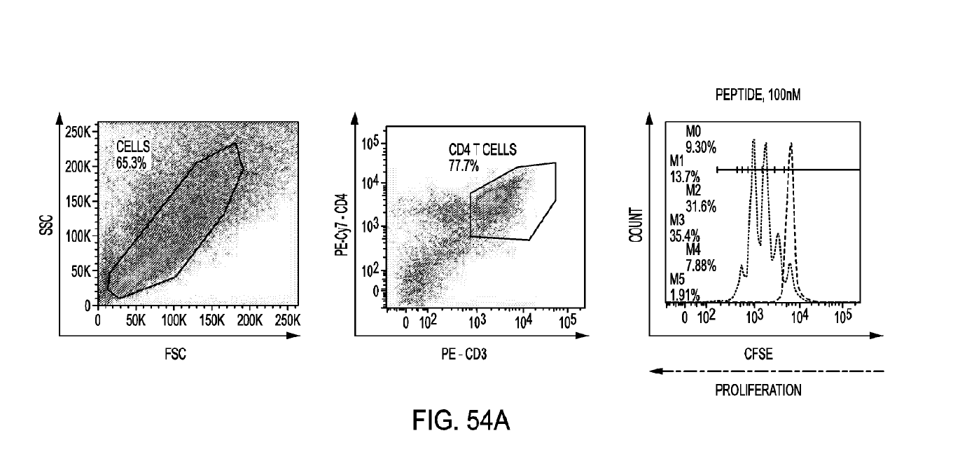

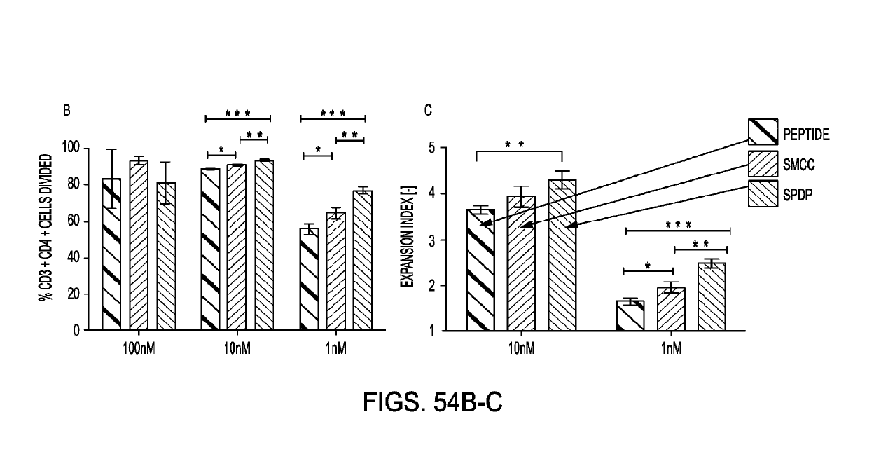

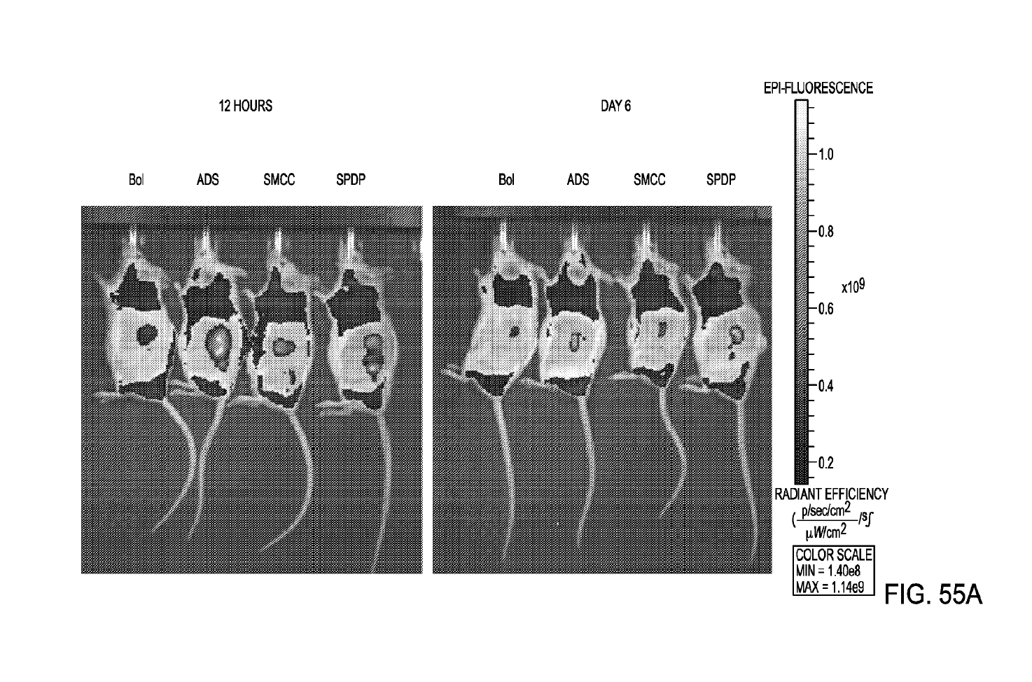

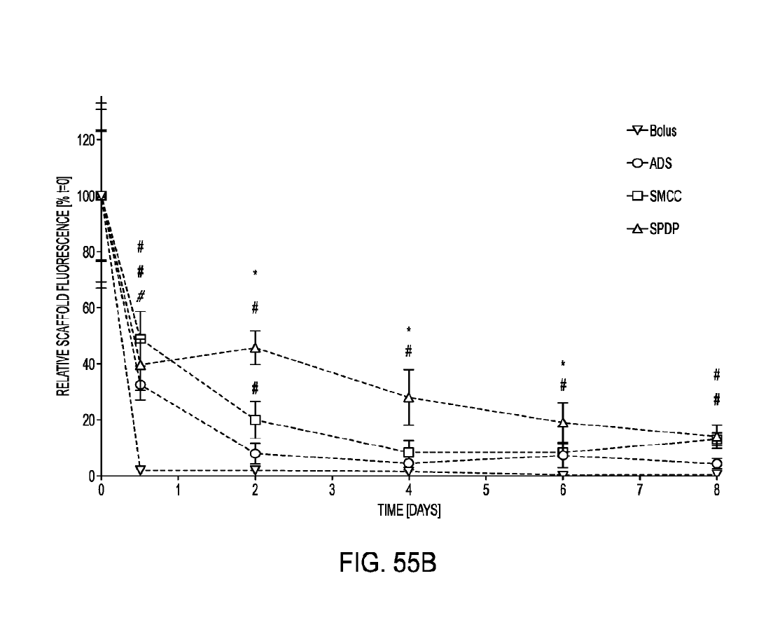

In non-limiting examples, a cysteine-containing peptide was conjugated to a MPS scaffold through stable maleimide (sulfhydryl-sulfhydryl), hereafter referred to as “SMCC”, or a reducible maleimide (sulfhydryl-sulfhydryl), hereafter referred to as “SPDP” linker. Two model peptides from OVA were used to demonstrate success of conjugation and enhanced presentation by APCs in vitro. See FIGS. 52-55. Additionally, prolonged local presentation of peptide conjugated to MPS was demonstrated compared to adsorption (i.e., associated with the structural material, e.g., MPS, but not actually covalently conjugated) and bolus (i.e., without a delivery device scaffold) formulations in vivo. See FIG. 55.

### Effects of Tolerogenic Immunoconjugates on T Cells

Adoptive transfer experiments, knockout animal studies, and drug trials have revealed the importance of T cells in immune activation disorders, including multiple sclerosis and the animal model experimental autoimmune encephalomyelitis (EAE). (D. R. Getts et al Immunotherapy 3, 853 (2011) and A. Jager, V. K. Kuchroo Scandinavian Journal of Immunology 72, 173 (2010)).

DC are critical regulators of T cell fate, and a principle mechanism for DC induced peripheral tolerance is through the modulation of T cell function. (D. Ganguly, et al. Nat. Rev. Immunol. 13, 566 (2013)).

The results presented herein demonstrate the ability of the compositions, e.g., immunoconjugates, described herein, to induce tolerance in T cells by attenuating T cell proliferation in vitro and by reducing disease severity in vivo (e.g., in an autoimmune disease model). DC treated with an immunoconjugate described herein reduced T cell responses in vitro and in vivo.

DC treated with a tolerogenic immunoconjugate were cultured with T cells, and T cell proliferation was monitored in vitro. For example, in vitro, the conjugate, e.g., dexamethasone-peptide (e.g., dexamethasone-SIINFEKL (SEQ ID NO: 9)) conjugate, inhibited T cell proliferation.

To examine the efficacy of an immunoconjugate in vivo, an immunoconjugate, e.g., dexamethasone-peptide (e.g., dexamethasone-MOG peptide) conjugate, was administered prophylactically, e.g., to EAE mice, and disease outcome was monitored. T cells had a reduced peptide-specific IL-17 elaboration. For example, adoptive transfer of splenocytes from animals treated with immunoconjugate resulted in limited protection.

As described in the results herein, the difference in health between the EAE animals in the free and conjugated tolerogen (e.g., dexamethasone) groups highlights the benefits of linking together the tolerogen (e.g., steroid, such as dexamethasone) with antigen (e.g., peptide such as MOG peptide). Covalently coupling the antigen and tolerogen limited off-target effects. In some embodiments, a tolerogen, e.g., dexamethasone, is modified, e.g., derivatized, and/or an immunoconjugate is designed, such that the physical and chemical properties affect its biodistribution, half-life, trafficking, and/or cellular-uptake, e.g., reduced uptake in cells with limited endocytosis, thereby limiting off-target effects.

Methods that enriched for DC and delivered the immunoconjugate enhanced disease outcomes, e.g., EAE outcomes, e.g., attenuated disease severity. Such strategies included prophylactically administering delivery devices, e.g., polymers such as poly (lactide-co-glycolide) materials, containing GM-CSF and tolerogenic immunoconjugate to diseased subjects, e.g., EAE animals.

EAE, an art-recognized model for multiple sclerosis, is a CD4+ T cell driven disease. The compositions described herein are suitable for treating EAE as well as other diseases of pathogenic CD4+T activation, e.g., allergy, rheumatoid arthritis, and lupus. Type 1 diabetes requires D4 and CD8 as does transplant rejection.

### Immune Activation Disorders

An immune activation disorder arises from aberrant or undesired immune activation. Examples of immune activation disorders include autoimmune diseases, allergies, asthma, and transplant rejection. The compositions described herein are useful to reduce the severity and/or frequency of an immune activation disorder. Immune activation disorders result from immunopathological responses directed against self and/or foreign antigens.

### Autoimmune Disorders

In an autoimmune disorder, the body mounts an abnormal immune response against a self antigen, e.g., a molecule, such as protein, peptide, nucleic acid, lipid, and/or carbohydrate normally present in the body.

Examples of autoimmune disorders include, e.g., multiple sclerosis, type 1 diabetes mellitus, Crohn's disease, rheumatoid arthritis, systemic lupus erythematosus, scleroderma, alopecia areata, antiphospholipid antibody syndrome, autoimmune hepatitis, celiac disease, Graves' disease, Guillain-Barre syndrome, Hashimoto's disease, hemolytic anemia, idiopathic thrombocytopenic purpura, inflammatory bowel disease, ulcerative colitis, inflammatory myopathies, polymyositis, myasthenia gravis, primary biliary cirrhosis, psoriasis, Sjögren's syndrome, vitiligo, gout, atopic dermatitis, acne vulgaris, and autoimmune pancreatitis.

For example, multiple sclerosis is thought to result from an immune response against the myelin sheath, which is normally important for mediating communication through the nervous system. For example, in multiple sclerosis, antibodies are made against proteins involved in myelination, such as MOG and MBP. Attack of myelination proteins leads to demyelination.

Risk factors for MS include an age between 15 and 60, female, a family history of MS, having or had an Epstein-Barr viral infection, having Northern European ancestry, having a thyroid disease, type 1 diabetes, or inflammatory bowel disease, and smoking.

MS is diagnosed by standard methods, e.g., blood tests, spinal tap, and/or magnetic resonance imaging (MRI) to detect lesions in the brain or spinal cord.

There are four clinical classes of diabetes: Type 1, Type 2, gestational, and diabetes due to other causes. Type 1 diabetes results from destruction of beta cells in the pancreas, typically leading to insulin deficiency. Type 2 diabetes is characterized by insulin resistance or hyperinsulinemia and patients often develop a progressive defect in insulin secretion. Gestational diabetes is characterized by glucose intolerance during pregnancy. Other types diabetes are due to or associated with other causes, e.g., genetic defects in insulin activity (e.g., genetic defects in the insulin receptor), pancreatic disease, hormonal diseases, genetic defects of beta cell function, or drug/chemical exposure. See, e.g., “Standards of Medical Care in Diabetes—2013.” Diabetes Care. 36.S1(2013):S11-S66; and Harris. “Classification, Diagnostic Criteria, and Screening for Diabetes.” Diabetes in America. National Institutes of Health, NIH Publication No. 95-1468. Chapter 2 (1995):15-36, incorporated herein by reference.

Diagnosis of diabetics includes the following criteria: a hemoglobin A1C (A1C) level of 6.5% or higher, a fasting plasma glucose (FPG) concentration of 126 mg/dL or greater, a 2-h plasma glucose concentration of 200 mg/dL or greater during an oral glucose tolerance test (OGTT), or for subjects having symptoms of hyperglycemia or hyperglycemic crisis, a random plasma glucose concentration of 200 mg/dL or greater. Fasting is normally defined as no caloric intake for at least 8 hours prior to testing. These tests are performed under conditions and standards generally known in the art, e.g., recommended by the World Health Organization and/or American Diabetes Association. See, e.g., “Standards of Medical Care in Diabetes—2013.” Diabetes Care. 36.S1(2013):S11-S66, incorporated herein by reference.

### Allergies and Asthma

Allergies are a body's heightened immune response to a foreign antigen, i.e., an allergen. For example, upon exposure of a T cell to an allergen, B cells produce allergen-specific immunoglobulin E (IgE) antibodies. In some cases, these IgEs bind to the surface of a mast cell, which triggers the release of inflammatory substances, such as histamine, prostaglandins, and leukotrienes and begins a cascade of inflammatory events that causes the allergic symptoms.

Examples of allergic conditions include latex allergy; allergy to ragweed, grass, tree pollen, and house dust mite; food allergy such as allergies to milk, eggs, peanuts, tree nuts (e.g., walnuts, almonds, cashews, pistachios, pecans), wheat, soy, fish, and shellfish; hay fever; as well as allergies to companion animals, insects, e.g., bee venom/bee sting or mosquito sting.

In some subjects, the inflammatory responses to an allergen lead to bronchial constriction/chest tightness, coughing, shortness of breath/rapid breathing, and/or wheezing-symptoms of allergic asthma. Allergic asthma is characterized by airway obstruction and inflammation. In some cases, allergic asthma is triggered by allergens such as dust mites, pet dander, pollen, and mold.

### Transplant Rejection

Transplantation of cells, tissues, or organs is performed to replace diseased or damaged cells, tissues, or organs with healthy ones. For example, transplantation of a cell, e.g., stem cell, such as hematopoietic stem cell, mesenchymal stem cell, peripheral blood stem cell, blood cell, bone marrow cell, or umbilical cord blood cell, replaces a damaged or diseased cell, e.g., in a patient who is suffering from or has suffered a chemotherapy, a radiation therapy, a cancer, a blood disorder (e.g., leukemia, lymphoma, multiple myeloma, or sickle cell anemia).

In organ transplantation, an organ (e.g., kidney, pancreas, heart, lung, liver, intestine, or thymus) from a healthy person replaces the organ in the diseased/injury host. Tissues, such as heart valves, cornea, skin, muscle tissue, bony tissue, and tendons, can also be transplanted.

In some situations, transplants use cells/tissues/organs from the host's own body (autologous), and in other cases, transplants use cells/tissues/organs from a donor of the same species (allogeneic) or an identical twin (syngeneic).

In some cases, transplantation is unsuccessful because of rejection by the host immune system of the replacement cells, tissues, or organs. Rejection is due to an immune response to foreign antigens on the transplanted cells, tissue, or organ (e.g., graft).

In cases where the donor and host are members of the same species, alloantigens are proteins/peptides that are different between the donor and the host, and are thus perceived as foreign by the host immune system.

Methods of preventing or reducing the severity of an immune activation disorder described herein comprising administering a composition (e.g., tolerogenic immunoconjugate) described herein to a subject are provided. In some embodiments, the subject suffers from or is at risk of suffering from an immune activation disorder. In some cases, the composition is administered to the subject prior to onset of an immune activation disorder. In other cases, the composition is administered while the subject is experiencing a symptom of an immune activation disorder. For example, the composition is administered after initial onset of an immune activation disorder.

For example, a composition described herein is suitable for use as a vaccine against an immune activation disorder.

One exemplary method described herein includes administration of a composition described herein in addition to administration of an immunomodulator drug, e.g., Glatiramer acetate (also called Copaxone®). For example, the composition described herein enhances the immunomodulatory effects (e.g., immune tolerance triggering effects) of the immunomodulator drug.

Uses of a composition described herein in the preparation of a medicament for preventing or reducing the severity of an immune activation disorder are also provided.

Materials and methods used to make and characterize the tolerogenic immunoconjugates, e.g., in Examples 1-4, are presented below.

### Flow Cytometry

Flow cytometry was conducted according to standard protocols. Cells were harvested, washed, and resuspended to a final cell concentration of 1-5 million cells/ml in ice cold phosphate buffered saline (PBS) with 10% fetal bovine serum (FBS) and 1% sodium azide. Anti-mouse antibodies including anti-CD11c, MHC II, CD80, CD86, and OVA 257-264 bound to H-2Kb were then aliquotted according to the manufacturer's recommended dilutions (Ebioscience). During staining, cells were incubated for 20 minutes at room temperature and then 20 minutes on ice. The cells were then washed and kept on ice until analysis. Some samples were fixed in 1% paraformaldehyde (PFA) for later flow cytometric studies. Flow cytometry was conducted on the BD LSR II or the BD Fortessa. Analysis was done using Flowjo (Tree Star Inc.).

### Mixed Leukocyte Reaction

The mixed leukocyte reaction was conducted according to previous Jaws II protocols (C. Haase, et al. Scandinavian Journal of Immunology 59, 237 (2004); and T. N. Jorgensen, et al. Scandinavian Journal of Immunology 56, 492 (2002)) and as described elsewhere. (A. Kruisbeek, et al. Proliferative Assays for T Cell Function, (2004)). Specifically, stimulator cells (either Jaws II cells or BMDC) were prepared in a cell suspension at a concentration of 5×10⁷ cells/ml in PBS with 25 μg/ml mitomycin C (Sigma) and incubated on a rocker for 20-25 minutes at 37° C. The cells were washed and plated in 96 well plates in 100 μl of supplemented RPMI-1640. Responder cells (splenocytes, T cells, or the D10.G4.1 cell line) were added to the stimulator cells in 100 μl at a ratio and number determined by a preliminary optimization experiment for the desired conditions and cell types (for optimization see (A. Kruisbeek, et al. Proliferative Assays for T Cell Function, (2004)). After two days, 0.5 μCi of [³H]-thymidine (New England Nuclear or PerkinElmer) were added to the cells for 18 hours. The cells were washed and rinsed with 5% cold trichloroacetic acid and left on ice for 30 minutes. The samples were then centrifuged at 3000 rpm at 4° C. for 6 minutes. The pellet was solubilized in 1 ml double distilled H₂O (ddH₂O) and 0.5 ml 10.25 N NaOH. The solution was then added to 13.5 ml Ultima Gold XR (PerkinElmer) and radioactivity was measured using the Tri-Carb 2800TR liquid scintillation counter (PerkinElmer). The Jaws II and D10.G4.1 cell lines were purchased from ATCC.

### Transwell Migration Experiments

To evaluate dendritic cell migration toward dexamethasone (Sigma-Aldrich), CCL19 (Peprotech) and CCL20 (Peprotech), 225,000 Jaws II cells were seeded onto 6 well transwell plates (Costar) with a 6 μm diameter membrane. The bottom of the well was supplemented with different doses of dexamethasone and migration was evaluated after 15-24 hours using a Coulter Counter (BD). Data was normalized to the average number of cells that migrated during an experiment and n=6-8.

### Peptide Synthesis and Purification

The peptide synthesis protocol was adapted from previous work (219). Reagents were obtained from Novabiochem (amino acids), Advanced ChemTech (N-methylpyrrolidone (NMP), N,N′-Diisopropylethylamine (DIPEA), piperidine, and trifluoroacetic acid (TFA)), Peptides International (N,N,N′,N′-tetramethyluronium hexafluorophosphate (HBTU), resin, and amino acids), Steraloids (dexamethasone hemisuccinate), and Sigma-Aldrich (all other reagents). Dexamethasone-SIINFEKL (SEQ ID NO: 9) synthesis was completed on a leucine pre-loaded 2-chlorotrityl resin at the 0.15-0.45 m equivalent scale. All amino acids were double coupled at 4× stoichiometry except for dexamethasone hemisuccinate (2×). Coupling was completed on a CS Bio CS336 X Peptide Synthesizer. Active sites were exposed with 2×15 minute 20% piperidine cleavage in NMP. Samples were activated with DIPEA and HBTU. At the end of the synthesis the resin was washed with NMP, dichloromethane (DCM), and methanol two times, dried, and treated with 50% TFA in DCM for 1.5 hours. A RotoVap® was used to concentrate the product and the sample was precipitated with cold diethyl ether. Purification was completed on an Agilent 1100 series reverse phase-high performance liquid chromatograph (RP-HPLC) using a C-18 column and analyzed on the LC-MS 1290/6140 (Agilent). A similar synthetic approach was followed to synthesize dexamethasone-MOG and dexamethasone-tyrosinase-related protein 2 (TRP2). For murine experiments, dexamethasone-MOG was used.

### MHC II and Co-Stimulatory Molecule Surface Presentation

Day 9 BMDC were harvested from 100 mm diameter plates and seeded into 6 wells of a tissue culture plate in R10 media containing 100 nM dexamethasone or dexamethasone-SIINFEKL (D-SIINFEKL (SEQ ID NO: 9)). The next day, the cells were treated with 50 ng/ml lipopolysaccharide (LPS) (Sigma), and the following day, the cells were harvested and stained according to the “Direct Staining Protocol” of Abcam with antibodies to MHC-II, CD80, CD86 or their respective isotype controls (Ebioscience). The BD LSR Fortessa was used to analyze the cells. Histograms were created using Flowjo (Tree Star Inc.) and statistical analysis was done using InStat (GraphPad Software).

### Interleukin-12 (IL-12) Elaboration

BMDC treatment was the same as described above for MHC II and co-stimulatory molecule assessment. On day 11, supernatants were aspirated and assayed by ELISA (IL-12 p⁷⁰ quantikine kit, R&D Systems) following the manufacturer's protocol. Statistical inference was completed using InStat (GraphPad Software) and the results were plotted in Excel 2007 (Microsoft).

### SIINFEKL (SEQ ID NO: 9) Antigen Presentation

SIINFEKL (SEQ ID NO: 9) is an ovalbumin derived peptide. Day 12 BMDCs were pulsed for 2 hours at 37° C. with 0 μM SIINFEKL (SEQ ID NO: 9), 3 μM SIINFEKL (SEQ ID NO: 9) (Peptides International), 3 μM SIINFEKL (SEQ ID NO: 9) plus 3 NM dexamethasone-SIINFEKL (SEQ ID NO: 9) (dexamethasone coupled to the antigen SIINFEKL (SEQ ID NO: 9)), or 3 μM dexamethasone-SIINFEKL (SEQ ID NO: 9) alone. Following washing, the cells were stained with anti-mouse SIINFEKL (SEQ ID NO: 9) antibody bound to the H-2Kb MHC class I alloantigen (H2Kb) (Ebiosciences) conjugated to R-phycoerythrin (PE) following the “Direct Staining Protocol” of Abcam and evaluated by flow cytometry on the BD LSR Fortessa. The samples were analyzed using FCS Express or FlowJo.

Materials and methods used to characterize the effects of tolerogenic immunoconjugates on T cells are described below.

### B3Z Cell: DC Co-Culture

BMDC were pulsed for 1 hour with SIINFEKL (SEQ ID NO: 9) or dexamethasone-SIINFEKL (SEQ ID NO: 9), washed, and 100,000 cells were plated in 200 p1 of R10 media at a 1:1 ratio with the B3Z T cell line. 15 hours later, the cells were fixed and stained with X-gal (Imgenix) according to the manufacturer's instructions. The cells were photographed at 10× magnification using a standard bright field microscope. A similar protocol was followed for the chlorophenol red-1-D-galactopyranoside staining assay (Imgenix), except that after 15 hours, the cells were washed and lysed in a chlorophenol red-β-D-galactopyranoside staining buffer. After a 4 hour incubation at 37° C., the absorbance at 590 nm was obtained.

### OT-I: DC Co-Culture

Cytotoxic T-lymphocytes (CTLs) were purified using magnetic beads (Miltenyi Biotec) from the spleens of the T cell receptor (TCR) transgenic OT-I mice (Jackson Laboratories) following the manufacturer's protocol. OT-I mice express a transgenic T cell receptor that recognizes ovalbumin residues 257-264 in the context of H2Kb. The CTLs were cultured with a 1:1 ratio with BMDC for 3 days at 37° C. in R10 media. Prior to co-culture, the BMDC were pre-treated for 1 hour with 1 μM dexamethasone-SIINFEKL (SEQ ID NO: 9) (or controls) and 0.01 μg/ml or 10.0 μg/ml ovalbumin (Sigma). The cells were washed two times before culturing with the T cells. Dexamethasone-TRP-2 was made following the same solid phase method as described above (Peptide synthesizer: CS Bio, DIPEA, Piperidine, TFA, and NMP: Advanced ChemTech, DCM: Sigma, amino acids and resin: Peptides International, dexamethasone hemisuccinate: Steraloids). Three days later, the cells were harvested and analyzed by flow cytometry using the BD LSR II Fortessa. The plots for flow cytometry were obtained using FCS Express.

### EAE Autoimmune Model

Female C57BL/6 (Jackson) mice (8-12 weeks) were left untreated (Untreated control), treated subcutaneously (s.c.) with MOG (200 μg) and dexamethasone (30 μg) in Incomplete Freund's Adjuvant (IFA) (D+MOG), or administered dexamethasone conjugated to MOG (240 μg, equimole to the MOG and dexamethasone applied alone) in IFA (D-MOG). Seven days later, disease was induced (day 0) by administering an injection of 250 μg MOG₃₅₋₅₅ s.c. (SynBioSci) in Complete Freund's Adjuvant (CFA) (Difco), and 200 ng of pertussis toxin (List Biological Laboratories) was given twice on consecutive days. The health of the animals was recorded for 1 month. The data was plotted in Excel (Windows) and analyzed with SPSS (IBM) and InSTAT (GraphPad Software) statistical programs. IFA and CFA are water-in-oil emulsions prepared from oils, such as paraffin oil and mannide monooleate. CFA contains killed Mycobacterium tuberculosis, while IFA does not.

### Dexamethasone and Immunoconjugate Quantitation

Dexamethasone and the immunoconjugate were quantitated by liquid chromatograph-mass spectrometry (LC-MS) or enzyme linked immunosorbent assay (ELISA). Compounds or standards were analyzed on an Agilent 1290 Infinity UPLC/6140 LC/MS on a Waters C18 reverse phase column with a gradient from (A) 0.1% trifluoroacetic acid (TFA) in water to (B) 95% acetonitrile, 9.9% H₂O, 0.1% TFA. Quantitation was completed by ultraviolet (UV) spectroscopy or total ionic current with appropriate standards. Alternatively, dexamethasone quantitation was completed using an ELISA kit from Neogen Corporation. In order to quantitate dexamethasone-peptide conjugates, samples were left overnight at 37° C. and analyzed the following day.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1: Inhibition of Dendritic Cell Activation and Proliferation with Dexamethasone

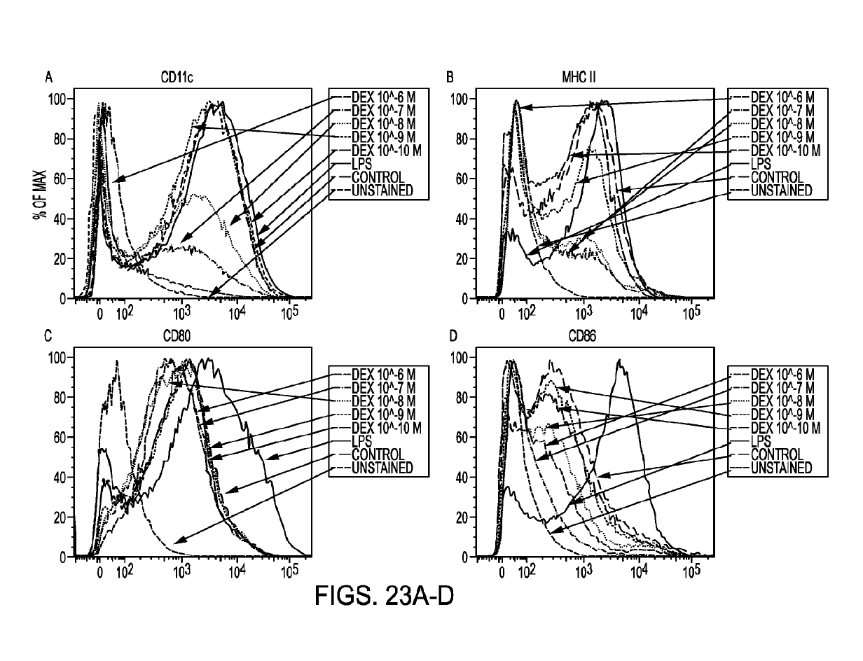

To determine the dose dependent effects of dexamethasone on DC and to establish a therapeutic window for treating DC, DC were treated with various concentrations of dexamethasone and the resulting phenotype was assayed. Dexamethasone treatment had a pronounced effect on DC phenotype and function (FIGS. 23A-D and 24A-E).

The histograms in FIGS. 23A-D demonstrate the effects of dexamethasone on the expression of CD11c (A), MHC II (B), CD80 (C), and CD86 (D) on primary bone marrow dendritic cells (BMDC) cultured for 10 days in vitro in the presence dexamethasone. The listed concentrations of dexamethasone were added on day 6 and day 8. For the lipopolysaccharide (LPS) condition, LPS was added to a concentration of 50 ng/ml on day 9. Control samples had no added dexamethasone. “LPS” samples had LPS, but no dexamethasone.

In immature bone marrow derived dendritic cell (BMDC) cultures (FIGS. 23A-D), treatment with dexamethasone inhibited the surface expression of MHC II and CD86 in a dose responsive manner with a more modest reduction in CD80 surface expression. Dexamethasone, particularly at concentrations of 10⁻⁸ M or higher, also reduced CD11c expression in the BMDC cultures.

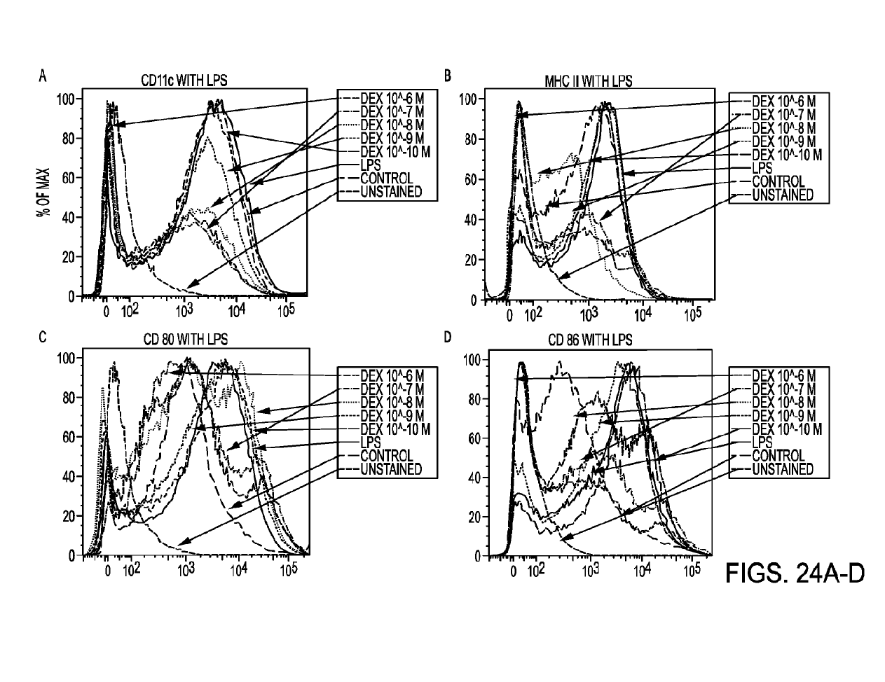

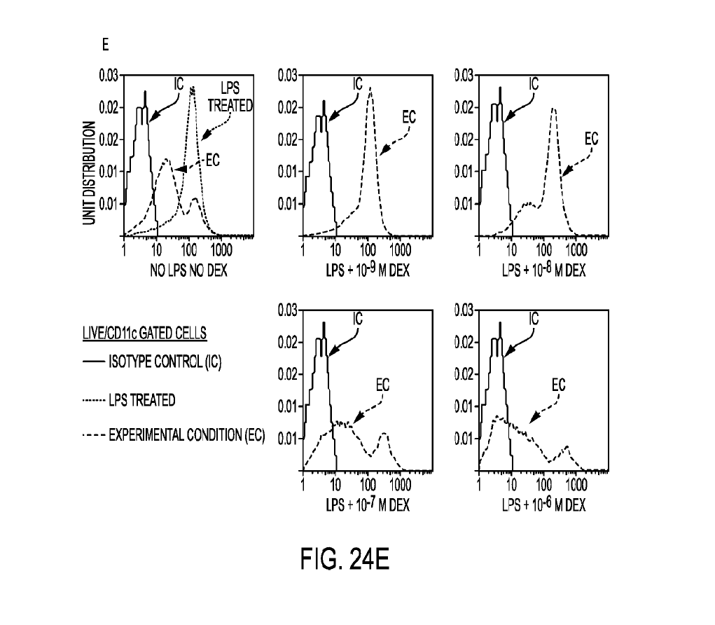

Analogous to results observed with immature DC, dexamethasone inhibited CD11c+ staining in a dose-responsive manner in mature DC treated with lipopolysaccharide (LPS) with effects beginning at dexamethasone concentrations around 10⁻⁸ M (FIGS. 24A-D). For these studies, dexamethasone was added on day 6 and day 8. LPS was added to a concentration of 50 ng/ml on day 9. Control samples had no added dexamethasone or LPS. “LPS” samples had LPS, but no dexamethasone. FIGS. 24A-D show the effects of dexamethasone on the expression of CD11c (A), MHC II (B), CD80 (C), and CD86 (D) on primary bone marrow dendritic cells grown for 10 days in vitro in the presence of LPS and dexamethasone. FIG. 24E shows a subset analysis of MHC II surface expression in CD11c+ gated cells.

Similar effects were observed with MHC II, CD80, and CD86 staining. For all conditions at concentrations greater than or equal to 10⁻⁸ M dexamethasone, staining was reduced compared to the LPS treated positive control.

Further, for both LPS treated and immature DC at dexamethasone concentrations of 10⁻⁸ M or greater, surface staining was reduced compared to untreated control cells, with the exception of CD80 and CD86 staining that was similar in cells treated with dexamethasone and LPS. Also, if dexamethasone and LPS treated cells were further gated on CD11c+ cells (FIG. 24E), a dose responsive decrease in MHC II surface expression was observed.

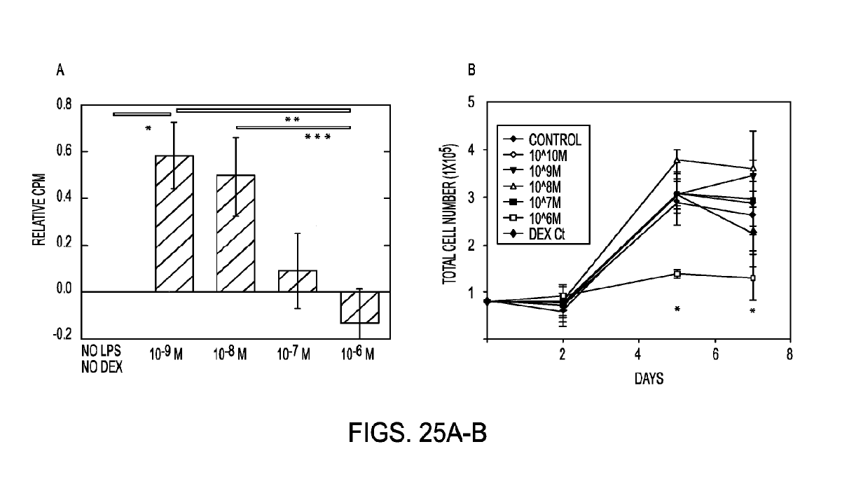

Building upon the phenotypic results shown in FIGS. 23A-D and 24A-E, functional assays were conducted to assess the ability of dexamethasone treated DC in blocking T cell proliferation (FIG. 25A). Dexamethasone and LPS (100 ng/ml) treated or untreated control (No LPS, no Dex) Jaws II cells were rendered incapable of dividing with mitomycin and were cultured with the D10.G4.1 T cell line. The uptake of tritiated thymidine was measured and plotted as the counts per minute normalized to the average cpm per experiment (4 experiments each with 4 samples) with a baseline set to the “No LPS No Dex” control condition (FIG. 25A). Dexamethasone treatment of DC cultured with LPS attenuated T cell proliferation in the mixed leukocyte reaction in a dose-responsive manner. Significant differences between groups containing untreated dendritic cells and dendritic cells treated with both dexamethasone and LPS were observed at concentrations lower than 10⁻⁸ M, while no difference was observed between the untreated group and the LPS and 10⁻⁷ M or 10⁻⁶ M dexamethasone treated groups. Moreover, there was an insignificant trend toward an even lower T cell proliferative response in the 10⁻⁶ M dexamethasone/LPS group compared to the untreated control group.

As glucocorticoids inhibit T cell proliferation, (A. E. Coutinho, et al. Molecular and Cellular Endocrinology 335, 2 (2011)) experiments were performed to determine whether dexamethasone could limit DC numbers (FIG. 25B). Jaws II cells were cultured in the presence of dexamethasone and the total cell number was enumerated over time (FIG. 25B). There were approximately one-half the number of DC in the group cultured with 10⁻⁶ M dexamethasone at day 5 than all of the other experimental groups including cells treated with 10⁻⁷ M dexamethasone. At day 7, a similar effect was observed and cells cultured in the presence of 10⁻⁶ M dexamethasone had approximately one-half the total number of cells in comparison to all other conditions (including the unmanipulated control cells, “control”) except for the Dex Ct group (cells treated with blank buffer vehicle) that showed a trend toward elevated cell numbers, but was not statistically different than the 10⁻⁶ M dexamethasone group. In sum, dexamethasone treated DC inhibited proliferation of T cells, and at high concentrations, dexamethasone reduced total DC number.

### Example 2: Dexamethasone had Minimal Effect on Dendritic Cell Migration

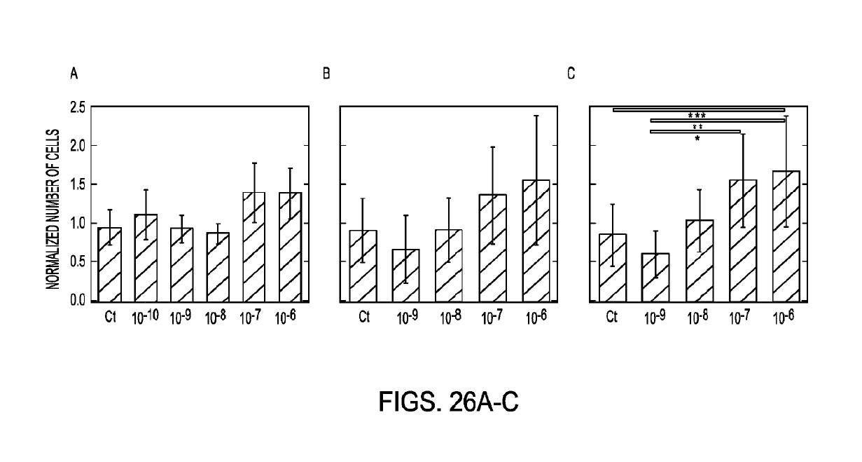

DC migration both to a vaccine site and then toward the draining lymph node of a vaccine is important in vaccine efficacy. As such, the effect of dexamethasone on DC migration was examined (FIGS. 26A-C). In transwell migration assays, dendritic cells showed a trend toward increased migration toward high concentrations of dexamethasone (FIG. 26A); however, this result was not significant at the 0.05 level. Similarly, cells treated with dexamethasone showed a trend toward greater migration to CCL19 than untreated controls; however, this result was not significant (FIG. 26B). In dendritic cells treated with 10⁻⁶ or 10⁻⁷ M dexamethasone, the number of cells that migrated to CCL20 was approximately 1.8 fold greater than in the control condition (FIG. 26C).

### Example 3: Design of a Dexamethasone Derivative for Antigen Specific Tolerance

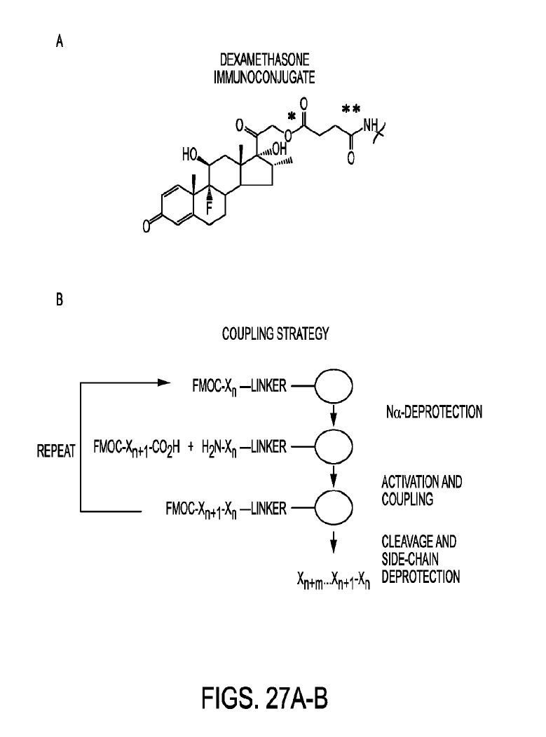

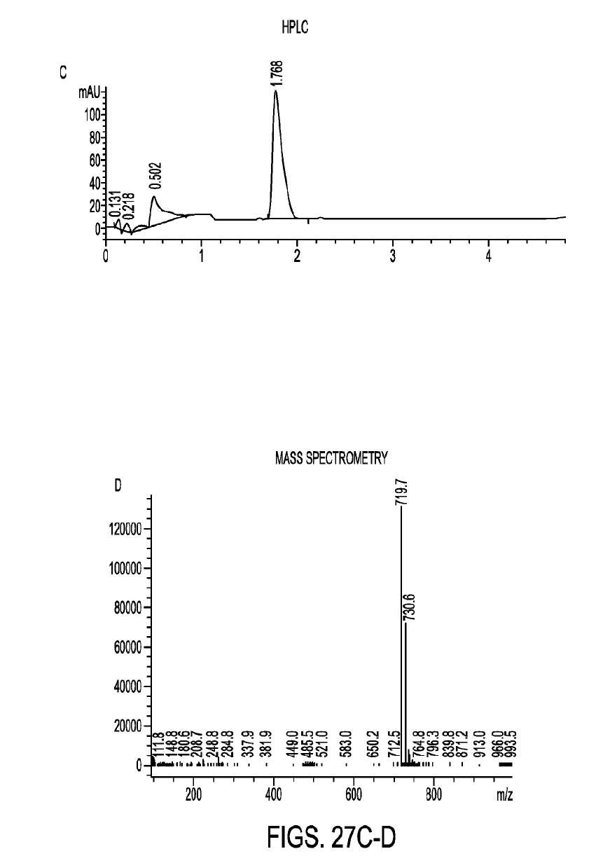

A synthetic strategy was designed for coupling dexamethasone to a generic peptide backbone (FIGS. 27A-D). In pharmaceutical preparations, dexamethasone can be derivatized with a phosphate at the primary alcohol on carbon 21, creating a more water soluble compound while still maintaining clinical potency. In this example, a derivitization strategy was selected such that the alcohol on carbon 21 of dexamethasone was covalently coupled to succinic anhydride. The resulting dexamethasone hemisuccinate (4-pregnadien-9α-fluoro-16α-methyl-11β, 17, 21-triol-3, 20-dione 21-hemisuccinate) was then chemically bonded to the N-terminus of a peptide (FIG. 27A) by standard solid-phase peptide synthesis (FIG. 27B).

Dexamethasone was coupled to the N-terminus of the SIINFEKL peptide chain, as evidenced by liquid chromatograph-mass spectrometry (LC-MS) (FIGS. 27C-D). The overall yield for the synthesis of Dex-SIINFEKL was 64%, and the purity by LC-MS at 210 nm (FIG. 27C) was 75%. The method was repeated for the synthesis of Dex-MOG₃₅₋₅₅ and Dex-TRP2 using traditional, non-labile, fmoc amino acids with a standard TFA cleavage cocktail.

For purification of the dexamethasone-peptide conjugate, in some embodiments, preparative, reverse-phase HPLC purification on a C18 column was performed to obtain a final product.

### Example 4: Inhibition of Dendritic Cell Activation with Peptide-Dexamethasone Immunoconjugates

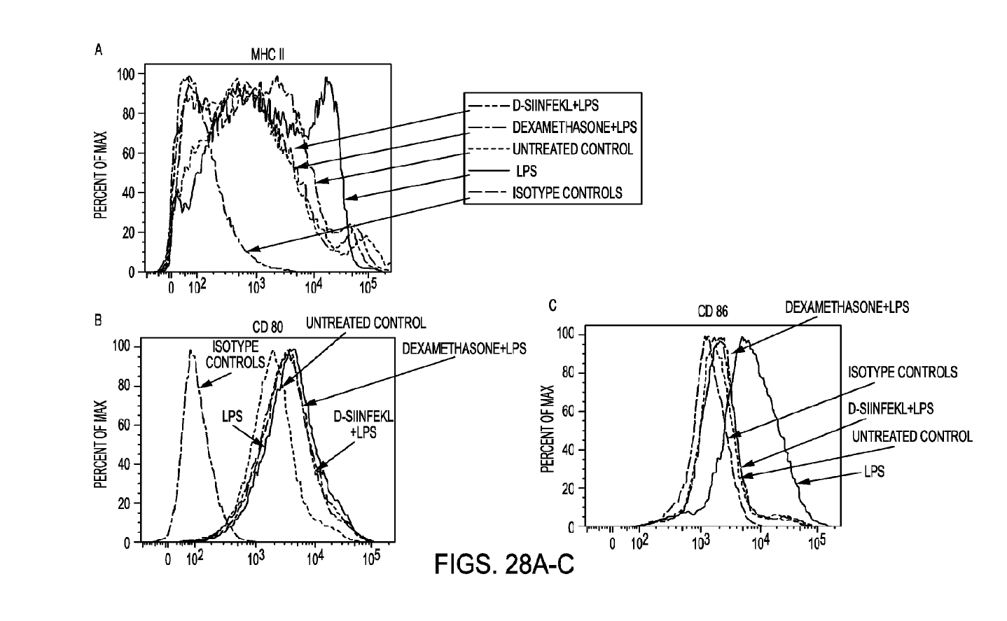

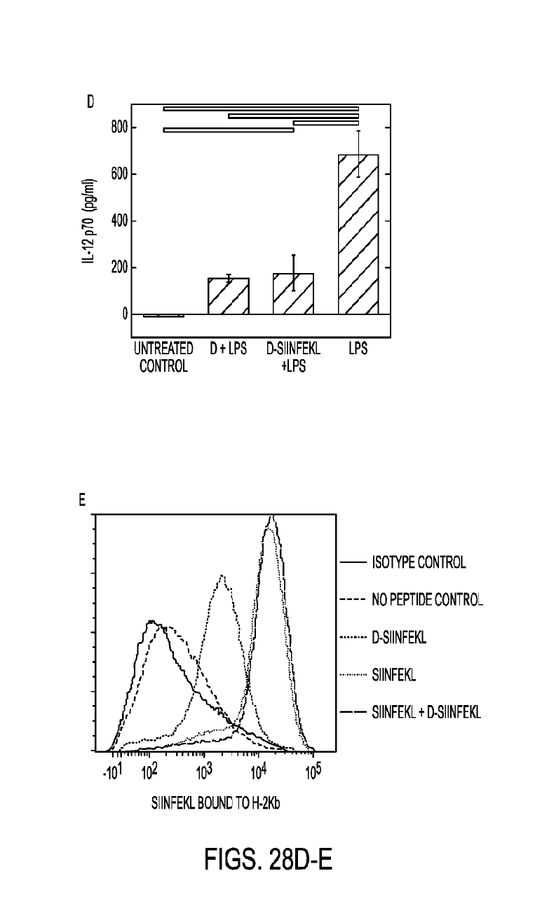

To ascertain whether both the tolerance inducing property of dexamethasone and antigen presentation of the peptide were preserved in the immunoconjugate, DC were treated with dexamethasone-SIINFEKL and assayed for the expression of tolerogenic markers and loading of peptide in the MHC I binding cleft (FIGS. 28A-E and Table 1).

BMDC were left untreated or were administered 100 nM dexamethasone or D-SIINFEKL. The next day, the cells were treated with 50 ng/ml LPS, and after an overnight culture, the cells were harvested. The surface expression of MHC II and the co-stimulatory molecules, CD80 and CD86, as well as the elaboration of IL-12p70, were examined (FIG. 28A-D).

TABLE 1
Average median fluorescence intensities (MFI) and the standard
deviations of groups depicted in FIG. 28A-C

| | MHC | | |
|---|---|---|---|
| MFI | II | CD86 | CD80 |
| Untreated | 1000 ± 100^{α} | 1900 ± 60^{β} | 1900 ± 100^{δ} |
| Control | | | |
| Dex + LPS | 540 ± 20^{α} | 2050 ± 5^{β} | 3100 ± 100^{δ} |
| Dex- | 580 ± 80^{α} | 2200 ± 200^{β} | 3300 ± 200^{δ} |
| SIINFEKL + LPS | | | |
| LPS | 1500 ± 200^{α} | 6200 ± 700^{β} | 4100 ± 200^{δ} |

^{α}p < 0.01 for all MHC II comparisons except for the Dexamethasone + LPS and D-SIINFEKL + LPS comparison (p > 0.05).
^{β}p < 0.001 in the CD86 group for the Untreated Control, Dexamethasone + LPS, and D-SIINFEKL + LPS groups compared to LPS group.
^{δ}p < 0.001 for all CD80 comparisons except for the Dexamethasone + LPS and D-SIINFEKL + LPS comparison (p > 0.05).
Statistical analysis completed using ANOVA with Tukey.

Like dexamethasone, dexamethasone-SIINFEKL inhibited the LPS induced increase in surface expression of MHC II, CD80, and CD86 (FIGS. 28A, B, and C, and Table 1). The median fluorescence intensity (MFI) of MHC II surface expression of the dexamethasone/LPS containing groups was nearly one-half that of the untreated, control cells and two-fifths that of LPS treated DC. The MFI of CD86 in dexamethasone/LPS treated samples was similar to that of untreated cells, and was approximately ⅓ that of LPS treated samples. The MFI of CD80 was elevated in the dexamethasone/LPS groups compared to untreated cells, and was three-fourths that of LPS treated DC. Culture with both dexamethasone and dexamethasone-SIINFEKL reduced the elaboration of IL-12 from BMDC by approximately a factor of 4 (FIG. 28D). In these assays, the potency of dexamethasone and the peptide conjugate were nearly equivalent.

To determine peptide loading from the conjugate onto MHC I, BMDC were pulsed for 2 hours with 0 μM SIINFEKL, 3 μM SIINFEKL, 3 μM SIINFEKL plus 3 μM dexamethasone-SIINFEKL, or 3 μM dexamethasone-SIINFEKL alone, washed, and stained with anti-mouse SIINFEKL bound to H2Kb (FIG. 28E).

The anti-mouse H-2Kb SIINFEKL antibody bound to the surface of DC pulsed with dexamethasone-SIINFEKL, as seen in the middle curve of the histogram (FIG. 28E); however, staining is substantially reduced compared to DC pulsed with an equivalent molarity of SIINFEKL alone. The amount of staining present in the SIINFEKL alone or the SIINFEKL plus dexamethasone-SIINFEKL groups was indistinguishable, reflecting that the presentation of dexamethasone-SIINFEKL did not inhibit SIINFEKL presentation (or antibody binding), or did so at a small amount that was not detectable with this assay.

### Example 5: Dendritic Cell Presentation of the Peptide-Dexamethasone Immunoconjugate to CD8+ T Cells In Vitro Attenuated T Cell Response and Reduced T Cell Proliferation

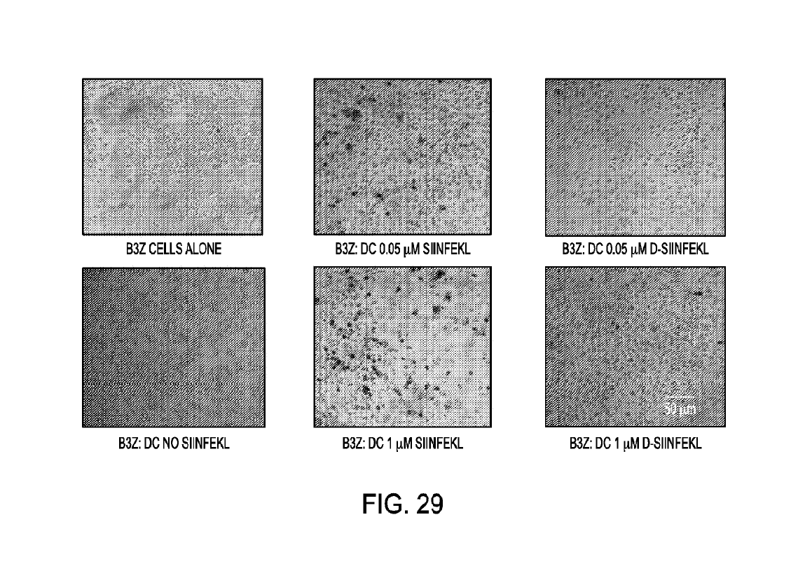

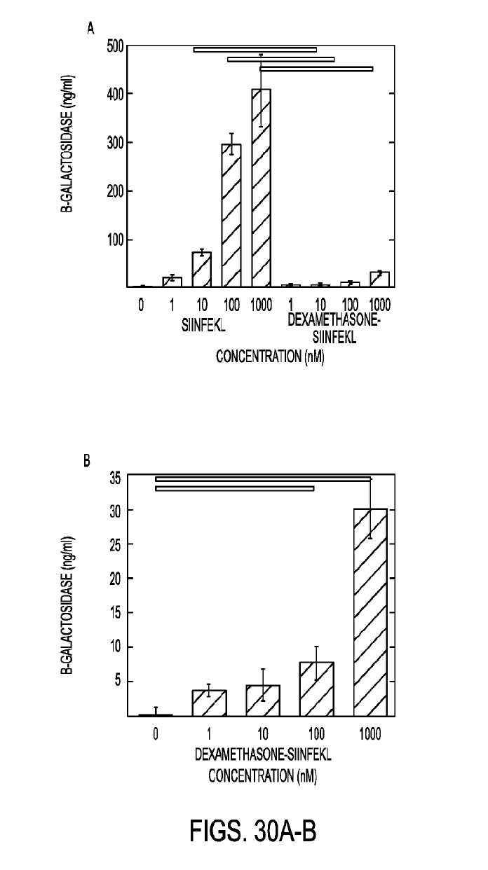

To evaluate dexamethasone-SIINFEKL antigen presentation to T cells, DC treated with the immunoconjugate or control peptide were cultured with the B3Z IL-2 reporter T cell line (FIGS. 29 and 30A-B). BMDC were pulsed for 1 hour with SIINFEKL or dexamethasone-SIINFEKL and were then cultured in equal numbers with the transgenic B3Z T cell line that recognizes SIINFEKL in the context of MHC Class I with the H-2Kb haplotype. Following a 15 hour co-culture, the cells were fixed, stained with X-gal, and imaged. The images were all obtained at 10× magnification and represent a typical distribution of cells (FIG. 29).

No staining was observed in the B3Z cells alone and the B3Z: DC No SIINFEKL controls, reflecting no BMDCs in the former and no antigen in the latter (FIG. 29). No staining was observed in the B3Z: DC 0.05 μM D-SIINFEKL groups. In contrast, the B3Z: DC 0.05 μM SIINFEKL group, with a similar quantity of peptide to the dexamethasone-SIINFEKL group, displayed many positive cells in a field of view. Also, at the 1.0 μM concentration of peptide, numerous positive cells were observed in the SIINFEKL group while only a few cells (fewer positive cells than the 0.05 μM SIINFEKL group) were positively stained in the dexamethasone-SIINFEKL group.

Using the same transgenic cells but a different reporter substrate, the qualitative results of FIG. 29 were confirmed in the quantitative results of FIGS. 30A-B. Specifically, BMDC were pulsed for 1 hour with SIINFEKL peptide or dexamethasone-SIINFEKL peptide conjugate and were then cultured for 15 hours with B3Z cells at a 1:1 ratio. The cells were then lysed and treated with the β-galactosidase substrate, chlorophenol red-β-D-galactopyranoside (CPRG). After 4 hours of incubation, the absorbance at 590 nm was obtained. Staining was due to β a-galactosidase expression driven by elements of the IL-2 promoter.

For the both the SIINFEKL and the dexamethasone-SIINFEKL groups, a direct dose-response relationship was observed between the amount of antigen and the amount of hydrolyzed CPRG. As the amount of antigen increased, so did the amount of staining. For antigen concentrations greater than 10 nM, the cells in the SIINFEKL groups exhibited greater CPRG hydrolysis. The groups treated with 100 nM and 1000 nM dexamethasone-SIINFEKL had signals greater than the control, untreated cells. A reduced amount of IL-2 expression was observed in B3Z cells co-cultured with BMDCs that had been treated with dexamethasone-SIINFEKL compared to SIINFEKL alone. These results demonstrated an attenuated T cell response in DC cultured with the immunoconjugate.

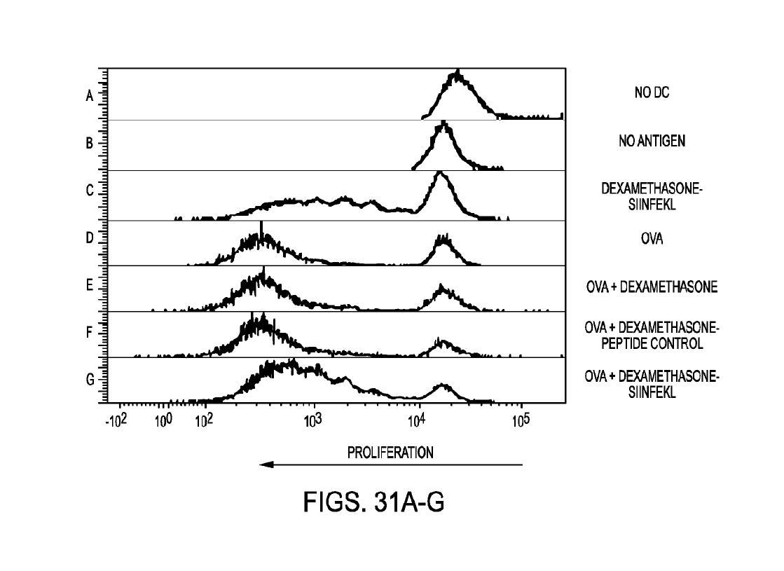

To further confirm these results, T cells isolated from OT-I mice were cultured with DC and T cell proliferation was monitored (FIG. 31).

Carboxyfluorescein succinimidyl ester (CFSE) labeled CD8+ T cells from TCR transgenic OT-I mice were cultured with BMDC for 3 days. Prior to co-culture, the BMDC were pre-treated for 1 hour with dexamethasone-SIINFEKL (or controls, as shown in FIG. 31) and thoroughly washed. Three days later, the cells were analyzed by flow cytometry.

Like the ovalbumin control (FIG. 31, row D), the dexamethasone-SIINFEKL immunoconjugate (FIG. 31, row C) was presented to T cells and initiated T cell proliferation. 0.2 μM ovalbumin at ⅕ the molarity (FIG. 31, row D) initiated a stronger proliferative response than dexamethasone-SIINFEKL at the 1.0 μM concentration (FIG. 31, row C). In the DC treated with ovalbumin, proliferation was unchanged when the DC were also pulsed with dexamethasone (FIG. 31, row E) or with the dexamethasone-irrelevant peptide control immunoconjugate (FIG. 31, row F). Unlike the dexamethasone or the dexamethasone-TRP2 treated groups, dexamethasone-SIINFEKL was able to reduce T cell proliferation in samples treated with ovalbumin (FIG. 31, row G).

### Example 6: Prophylactic Treatment with Dexamethasone-MOG₃₅₋₅₅ Attenuated Experimental Autoimmune Encephalomyelitis (EAE)

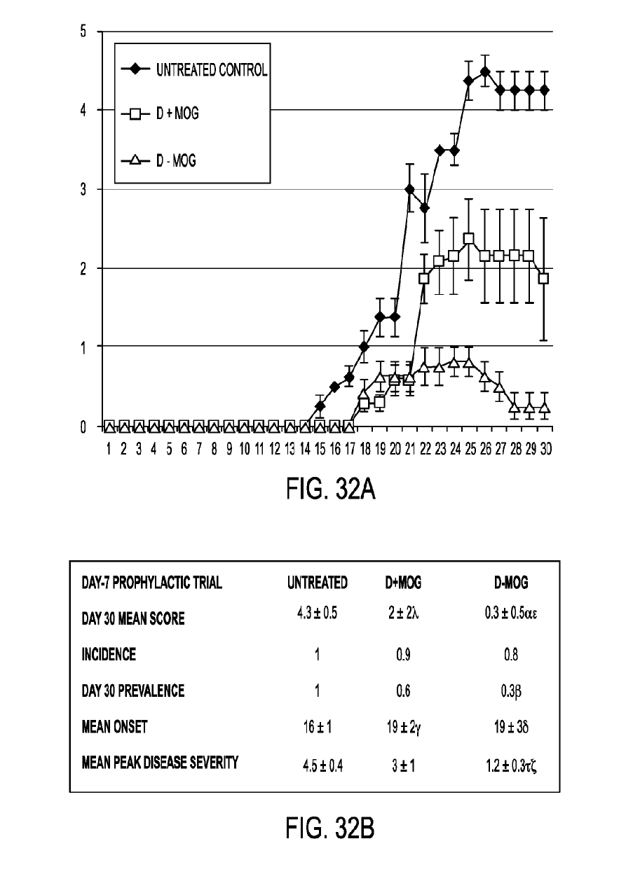

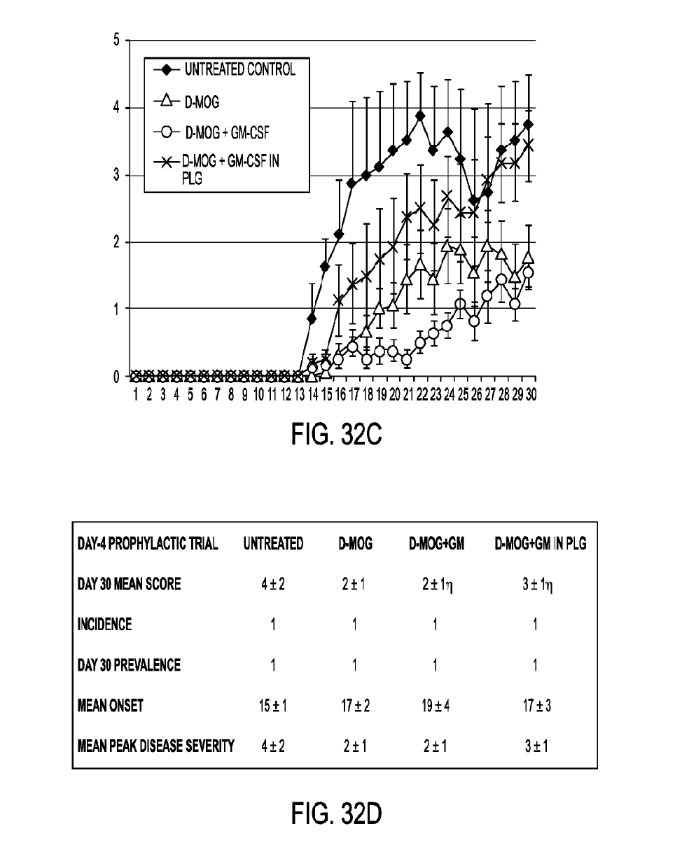

In order to examine the effects of the immunoconjugate on T cells in a T cell dependent disease, immunoconjugate was given prophylactically in an animal model of multiple sclerosis, experimental autoimmune encephalomyelitis (EAE) (FIGS. 32A-D).

A prophylactic trial in mouse models was conducted, in which C57BL/6 mice were left untreated (Untreated control), treated s.c. with MOG (200 μg) and dexamethasone (30 μg) in IFA (D+MOG), or treated with dexamethasone conjugated to MOG (240 μg, equimole to the MOG and dexamethasone applied alone) in IFA (D-MOG). Seven days later, disease was induced (day 0) and the animals were monitored for 1 month (FIG. 32A).

When the immunoconjugate was administered in IFA 7 days prior to the induction of disease, EAE disease onset was delayed and severity was attenuated (FIGS. 32A-B). Animals treated with either the dexamethasone-MOG immunoconjugate (D-MOG) or dexamethasone and MOG (not covalently coupled, D+MOG) both developed disease at later time points than untreated animals; however, only the immunoconjugate had a lower disease severity, disease prevalence, and mean peak disease severity in comparison to untreated animals. Further, the mean peak disease severity and the mean clinical score on day 30 were significantly lower for the D-MOG immunoconjugate group in comparison to the D+MOG treatment group.

As GM-CSF is a potent DC enrichment factor and DC are critical for inducing tolerogenic responses, experiments were conducted to determine if GM-CSF releasing materials could enhance tolerogenic responses when delivered with an immunoconjugate (FIGS. 32C-D).

Four days prior to EAE disease induction, animals were treated s.c. with a bolus of 100 μg of the immunoconjugate (D-MOG), a bolus of 100 μg of the immunoconjugate (D-MOG) with 3 μg of GM-CSF in PBS (D-MOG+GM), or 100 μg of the immunoconjugate and 3 μg of GM-CSF in a macroporous poly (lactide-co-glycolide) scaffold (D-MOG+GM in PLG). D-MOG was mixed with microspheres containing GM-CSF and sucrose with a porogen size between 250 μm and 425 μm and was gas-foamed as described in Ali et al. Sci. Transl. Med. 1.8(2009):8ra19. Four days later, EAE disease was induced.

There was a trend toward a reduced disease phenotype and a later disease onset between the D-MOG and D-MOG+GM groups in comparison to control animals. The more severe mean score at day 30 of D-MOG+GM in PLG treated animals in comparison to the bolus D-MOG+GM treated animals was significantly different at the 0.05 level. The D-MOG bolus delivery performed better than the D-MOG in the polymer scaffold.

### Example 7: Biomaterial Delivery of the Immunoconjugate

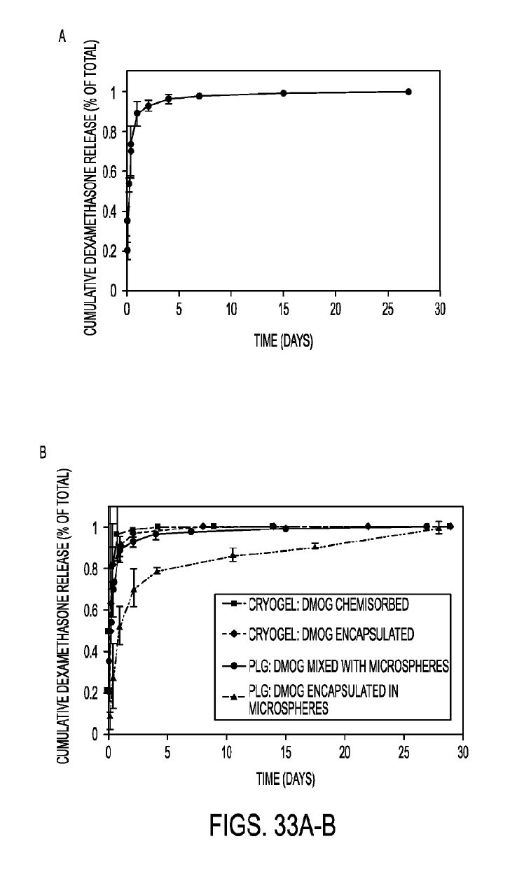

In order to further characterize the material system that contained GM-CSF and immunoconjugate and evaluate the level of immunoconjugate that was delivered throughout the EAE experiment, the release of the immunoconjugate was quantitated by monitoring dexamethasone concentrations (FIG. 33A).

Dexamethasone-peptide immunoconjugate delivery at 37° C. in PBS was quantitated by a dexamethasone ELISA over the course of a month (FIGS. 33A-B). Release of dexamethasone from PLG materials used in the EAE trial described in Example 6 was measured (FIG. 33A). 89%±6% of dexamethasone was released in the first day of implantation, and the overall encapsulation efficiency post sterilization was 12%±2. Sterilization occurred for 15 minutes after the scaffolds were synthesized.

To determine if other macroporous biomaterials that release GM-CSF and abundantly enrich for DC could be useful for vaccination (FIG. 33B), immunoconjugate release studies from three other scaffolds were completed: PLG scaffold with immunoconjugate loaded into the microparticles during the WOW (water in oil in water) emulsion step (DMOG Encapsulated in Microspheres), macroporous cryogel with the immunoconjugate chemisorbed (DMOG Chemisorbed), or macroporous cryogel with the immunoconjugate added to the polymerization cocktail (DMOG Encapsulated). Samples were placed on a rocker at 37° C. in PBS. Except for the PLG scaffold with Dex-MOG incorporated into the microparticles (PLG:DMOG Encapsulated in Microspheres), the majority of compound was released rapidly in the first 12 hours. For the PLG scaffold with Dex-MOG loaded within microspheres (PLG: DMOG Encapsulated in Microspheres), 52%±9% was released in the first day and thereafter gradually tapered. Therefore, for these PLG scaffolds, the overall release was characterized by an initial burst phase followed by the gradual release over the course of the month.

### Example 8: Hydrolysis of the Immunoconjugate

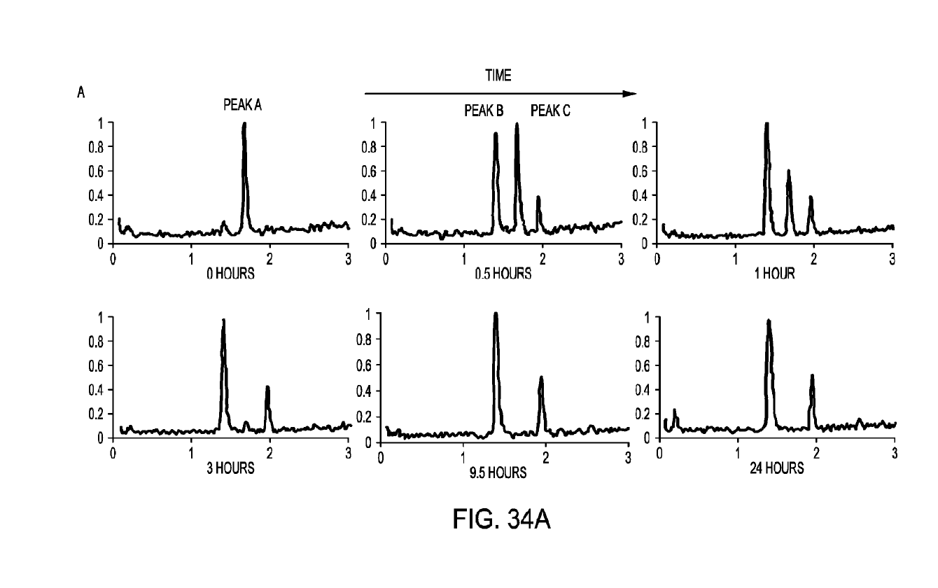

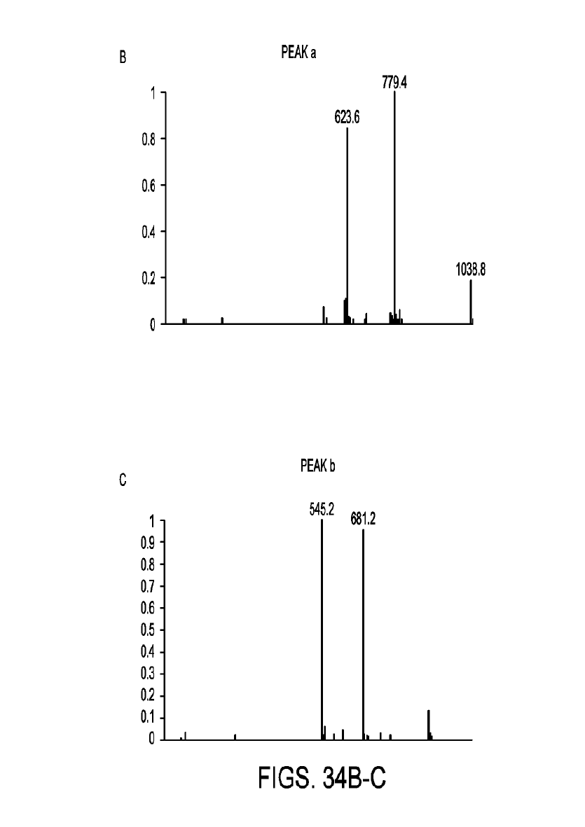

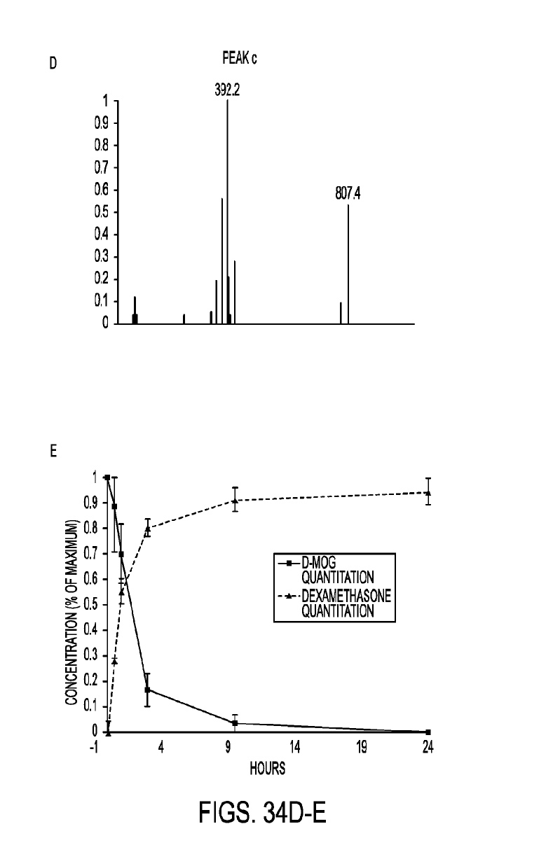

To evaluate the stability of the immunoconjugates empirically outside of the ester hydrolysis prediction model developed by the Environmental Protection Agency (see the Hydrowin v 2.00™—E.P. Agency. (2012), vol. 2013), ester hydrolysis of the dexamethasone-MOG immunoconjugate was evaluated at 37° C. in PBS at pH 7.4 (FIGS. 34A-E). Specifically, Dex-MOG was incubated in PBS at 37° C. and rapidly frozen for later LC-MS analysis.

After one-half hour at 37° C., peptide (peak b) and dexamethasone (peak c) peaks were visualized by LC-MS (FIG. 34A). Mass spectra, reflecting the mass to charge ratios of the entire immunoconjugate, peptide fragment, or dexamethasone molecule, respectively, were obtained. The mass spectra of peaks a (immunoconjugate), b (peptide fragment), and c (dexamethasone) are shown in FIGS. 34B-D. The quantitation and rate of dexamethasone formation and immunoconjugate scission is depicted in FIG. 34E. Assuming pseudo first order rate kinetics, the kd was 1.2×10⁻⁴±5×10⁻⁵ (s⁻¹) with a τ of 20±10 hours.

The hydrolysis of the immunoconjugate in the material was also investigated. Specifically, the stability of the immunoconjugate within PLG was determined by assessing the hydrolysis of the immunoconjugate released from PLG scaffolds at 4° C. At 4° C., in comparison to 37° C., hydrolysis was substantially retarded, as governed by the Arrhenius equation, while the diffusion constant changed minimally. The compound that was released at early time points from the material likely reflected the molecule within the scaffold, i.e., if dexamethasone and peptide were observed as separate components early on, then the immunoconjugate was likely cleaved within the material.

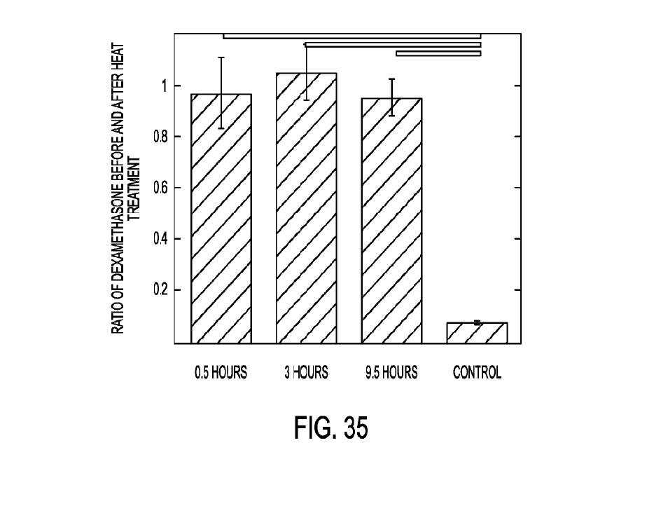

PLG scaffolds containing immunoconjugate prepared in the same manner as the scaffolds used in the EAE animal trials described above were placed in PBS at 4° C. on a rocker. Samples at different time points were collected and immediately frozen for ELISA analysis. The control sample reflected the control immunoconjugate not incorporated into the scaffold. After 0.5 hours, the immunoconjugate was completely fragmented into its constituent parts, while there was minimal fragmentation in the control immunoconjugate not incorporated into the scaffolds (FIG. 35). Thus, dexamethasone-MOG was degraded in the PLG scaffolds.

### Example 9: In Vivo T Cell Response to the Immunoconjugate

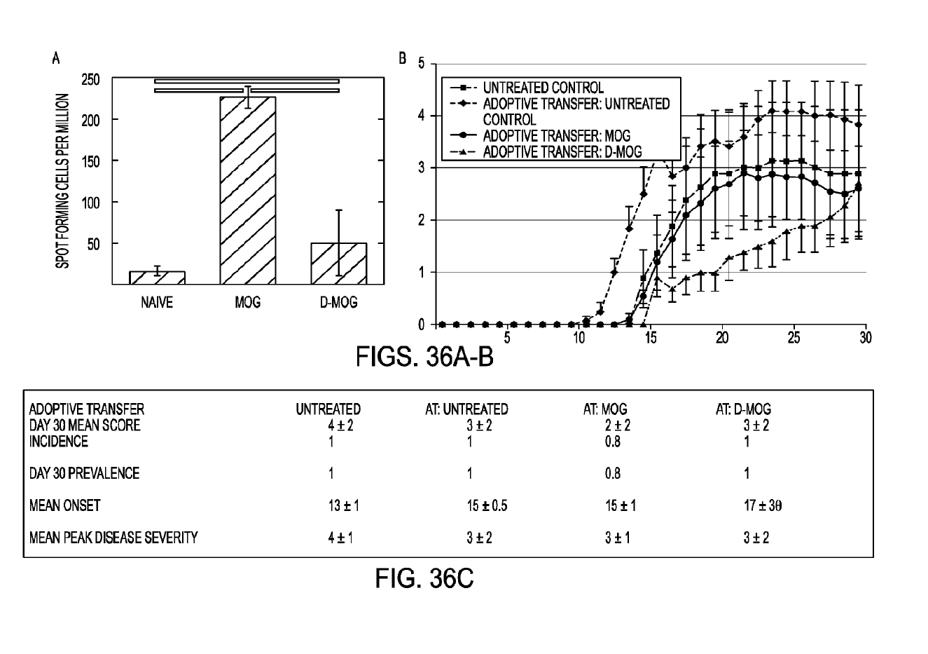

To further explore the mechanism for tolerance induction in EAE animals treated with bolus immunoconjugate, T cell analyses (ELISpot and passive EAE assays) were completed in mice that received the immunoconjugate or control therapies (FIG. 36A-C).

Splenocytes from mice treated with MOG alone or Dex-MOG with EAE induced were challenged with MOG to quantitate antigen specific elaboration of IL-17. Like naïve mice, the number of spot forming cells per million in the Th17 ELISpot assay was significantly reduced in the immunoconjugate group in comparison to the MOG alone group (50±40 spot forming cells per million compared to 230±10 spot forming cells per million) (FIG. 36A).

Splenocytes from diseased animals were transferred by tail vein injection into healthy (wild-type) mice (passive EAE model), and the severity of EAE was monitored. There is a delay in mean onset of disease from 13 to 17 days in the cells taken from immunoconjugate treated mice compared to controls with disease induced with MOG. The incidence, prevalence, mean peak disease severity, and day 30 mean score were similar among all of the groups (FIGS. 36B-C).

### Example 10: Antigen-Adjuvant Conjugates

Delivery of an antigen-adjuvant conjugate from the mesoporous silica (MPS) vaccine scaffold increases the immunogenicity and CD8 T cell response towards the antigen as compared to delivering the antigen and adjuvant as separate entities. An antigen was covalently conjugated to a TLR adjuvant through bifunctional maleimides (amine-sulfhydryl), carbodiimide (amine-carboxylic acid) and photo-click (norbornene-thiol) linkers. Success of conjugation and in vivo T cell responses were demonstrated using a model antigen Ovalbumin (OVA), its CD8 epitope SIINFEKL. SIINFEKL was used as a model antigen; however, the antigen could comprise a) a protein or peptide against which an immune response is sought to be elicited or b) a lysate of a cell associated with tumor. Other TLR agonists such as MPLA and Poly (I:C) and those listed above are optionally used to make antigen-adjuvant conjugates for vaccine purposes. CpG or poly I:C are optionally condensed. To condense the nucleic acids, the NH2 groups on the polyethyleneimine are functionalized with maleimide and conjugated to reduced thiol-CpG or other nucleic acid moieties.

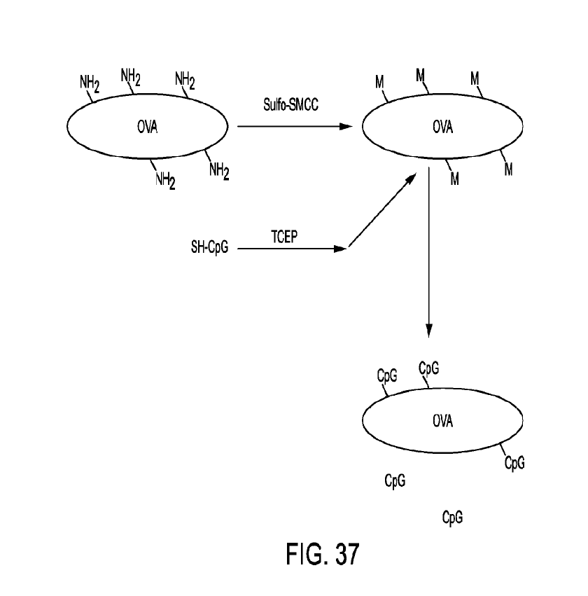

FIG. 37 shows a scheme of antigen-adjuvant conjugation. OVA protein at 5 mg/ml was reacted with 50 molar excess of sulfo-SMCC NHS (Pierce) in pH 7.5 PBS for 2 hours to functionalize primary amines on the protein with maleimide. After purification via 7K desalting column (Pierce), the modified protein was added to a solution of reduced thiol-CpG (IDT) containing 1 free thiol per CpG molecule and reacted on shaker for 12 hours at room temperature. Excess CpG was removed using a 30K spin filter column (Millipore). Similarly, cysteine containing peptides, such as CSIINFEKL, were conjugated to amine modified CpG (IDT).

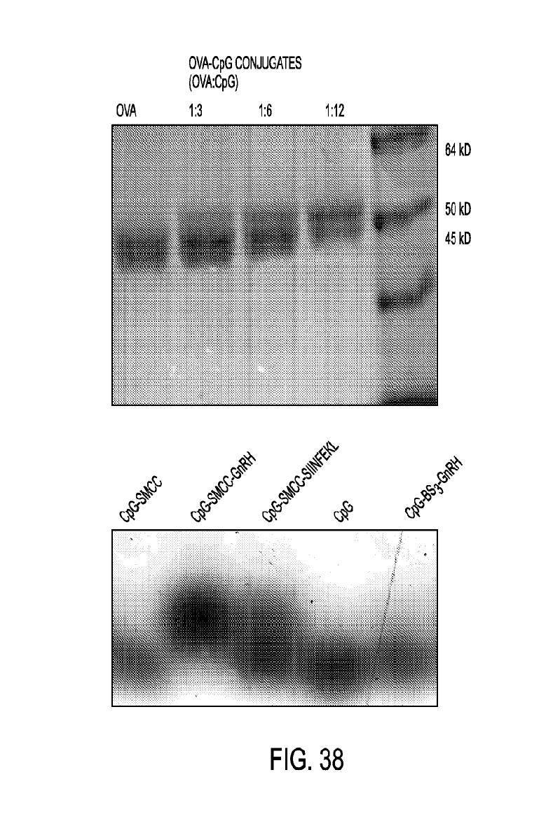

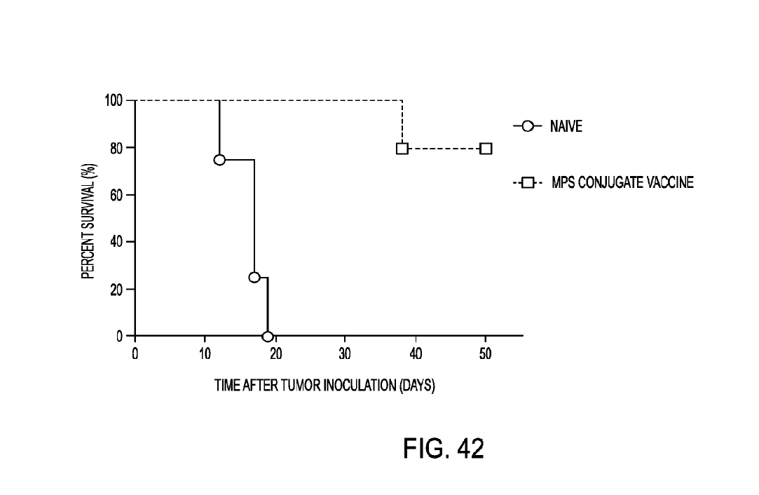

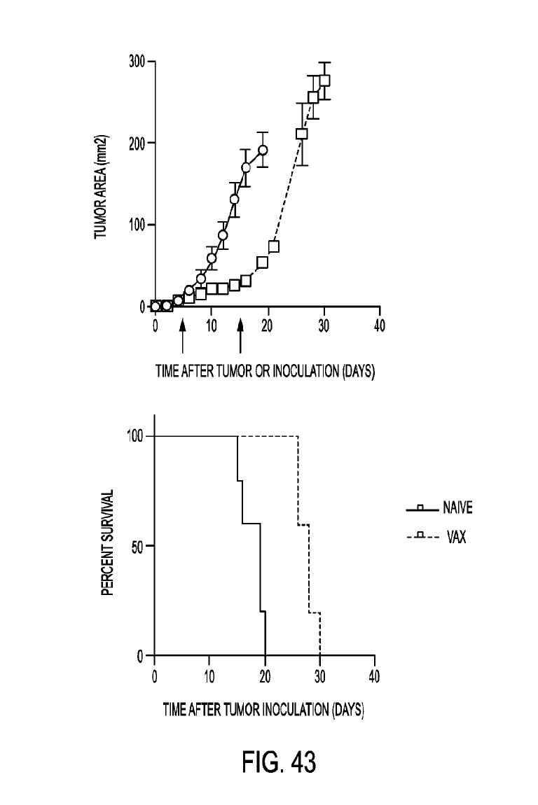

CpG-OVA conjugation was confirmed using gel electrophoresis (non-reducing, denaturing 10% Tris-Glycine) (FIG. 38, upper panel). On average, each OVA protein contains 1 CpG molecule (average for the whole OVA protein). Using the maleimide-thiol chemistry, roughly 1-2 CpGs are linked onto the OVA protein. However by changing the chemistry, the efficiency of the conjugation is increased. (FIGS. 42, 43).

CpG was conjugated onto CSIINFEKL (CD8 T cell epitope on OVA) and CEHWSYGLRPG (GnRH peptide) to increase the immunogenicity of the peptides and evoke potent antibody response against the peptide antigens. CpG-peptide conjugation was confirmed using gel electrophoresis (4% agarose). The additional bands at higher molecular weight indicate successful conjugation of CpG and GnRH (lane 2) or SIINFEKL (lane 4) using the maleimide linker. Using this reaction scheme, every CEHWSYGLRPG peptide contains 1 CpG molecule, whereas roughly 40% of the CSIINFEKL peptide is modified with 1 CpG molecule (FIG. 38, lower panel).

Conjugation of Poly(I:C) and MPLA to EHWSYGLRPG is also conjugated using carbodiimide chemistry. Phosphate groups of PolyIC and MPLA are first activated using excess EDC(1-ethyl-3-(3-dimethylaminopropyl)carbodiimide) in 0.1 M methylimidazole buffer (pH 7.5) for 2 h prior to addition of 5 equivalence of peptide antigens. The subsequent reaction is allowed to proceed for 12 h.

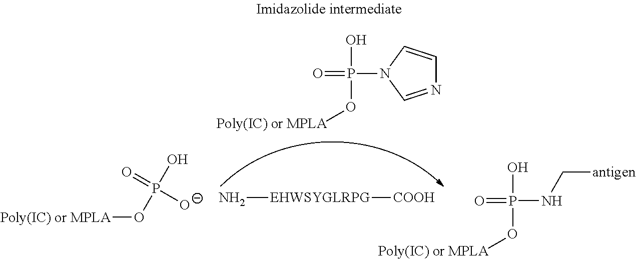

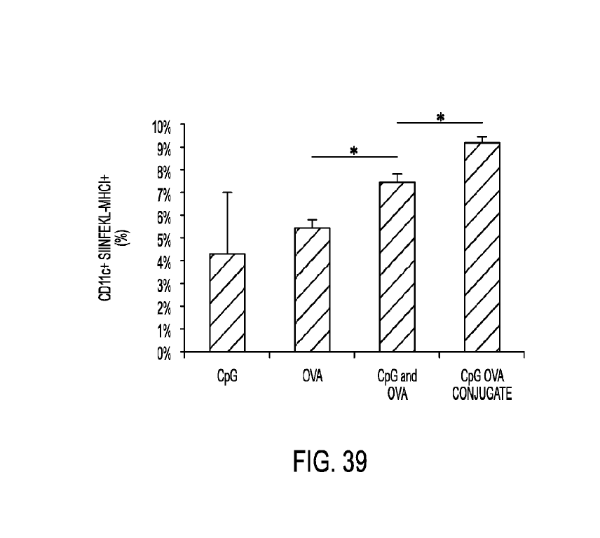

CpG-OVA conjugate was cultured with bone marrow derived dendritic cells (BMDC) in vitro for 18 hours. BMDC presentation of SIINFEKL was analyzed using flow cytometry and percentage of CD11c+ DCs presenting the SIINFEKL peptide on the MHC-I molecule was quantified. The CpG-OVA conjugate showed enhanced presentation as compared unconjugated CpG and OVA in vitro (FIG. 39).

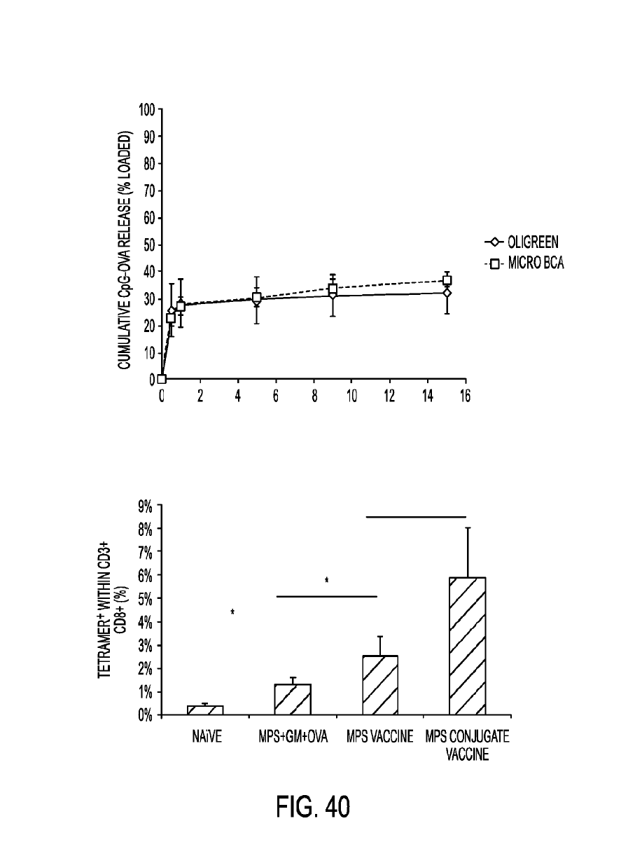

The CpG-OVA conjugate was loaded into the mesoporous silica (MPS) scaffold and was released in a sustained manner followed by a burst release (FIG. 40, top). C57bl/6J mice were immunized with MPS scaffold containing 1 ug GM-CSF and 100 ug OVA, 1 ug GM-CSF, 100 ug CpG and 100 ug OVA (MPS vaccine) or 1 ug GM-CSF and 100 ug OVA conjugated to 100 ug CpG (MPS conjugate vaccine). After 7 days, peripheral blood was analyzed using SIINFEKL tetramer and the percentage of SIINFEKL specific T cells within CD3+CD8+ T cells was quantified. MPS conjugate vaccine increased the presence of SIINFEKL specific CD8+ T cells by 2 fold compared with the MPS vaccine (FIG. 40, bottom).

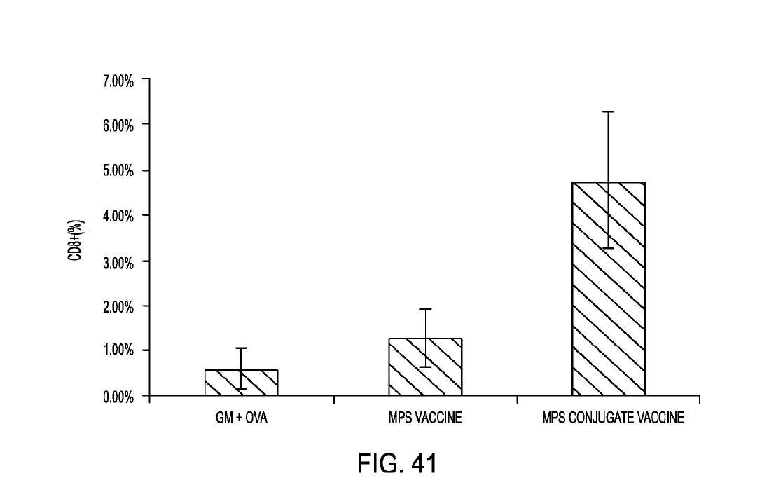

C57bl/6J mice were immunized with MPS scaffold containing 1 ug GM-CSF and 100 ug OVA, 1 ug GM-CSF, 100 ug CpG and 100 ug OVA (MPS vaccine) or 1 ug GM-CSF and 100 ug OVA conjugated to 100 ug CpG (MPS conjugate vaccine). After 11 days, the scaffold was explanted and analyzed for CD8 T cell infiltration. MPS conjugate vaccine enhanced significantly higher CD8 T cell homing to the scaffold than the unconjugated vaccine (FIG. 41).

C57bl/6J mice were immunized with MPS scaffold containing 1 ug GM-CSF and 100 ug OVA conjugated to 100 ug CpG (MPS conjugate vaccine. After 11 days, mice were inoculated with 3×10⁵ B16 melanoma cells transfected with the OVA vector (B16-OVA) and tumor growth was monitored. The MPS conjugate vaccine resulted in 80% prophylactic tumor protection whereas unvaccinated naïve mice succumbed to tumor within 20 days (FIG. 42).

The MPS conjugate vaccine was evaluated in a therapeutic model. C57bl6J mice were inoculated with 3×10⁵ B16 melanoma cells transfected with the OVA vector (B16-OVA). When tumor area reached ˜5 mm², mice were treated with 1 injection of the MPS conjugate vaccine (Vax). After vaccinating with the MPS conjugate vaccine, tumor growth was significantly slowed and animal survival was significantly prolonged (FIG. 43). These data indicate that immunization with a immunoconjugate containing an immunostimulatory agent and a tumor antigen [e.g., an antigen obtained from a tumor cell lysate derived from a patient biopsy or a recombinant tumor antigen] results in an increased anti-tumor response compared to immunization with an antigen that is not covalently conjugated to an immunostimulatory agent. Tumor antigen can be in the form of whole tumor cells (live, dead, or attenuated, e.g., irradiated), disrupted whole cells, e.g., a tumor cell lysate, or purified/isolated tumor antigen or mixtures of purified/isolated antigens.

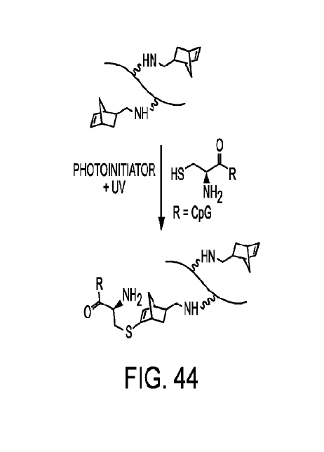

OVA protein at 5 mg/ml was reacted with 5-norbornene-2-acetic acid succinimidyl ester (Sigma-Aldrich) in 20 molar excess to functionalize primary amines on protein with norbornene. After purification via desalting column (Pierce) the modified protein was added to a solution of reduced CpG (IDT) containing 1 free thiol per CpG molecule and a final concentration of 0.5% w/v photoinitator (Irgacure-2959, Sigma-Aldrich). Reaction mixtures were mixed well and irradiated for at 365 nm for 10 minutes at 10 mW/cm² (FIG. 44).

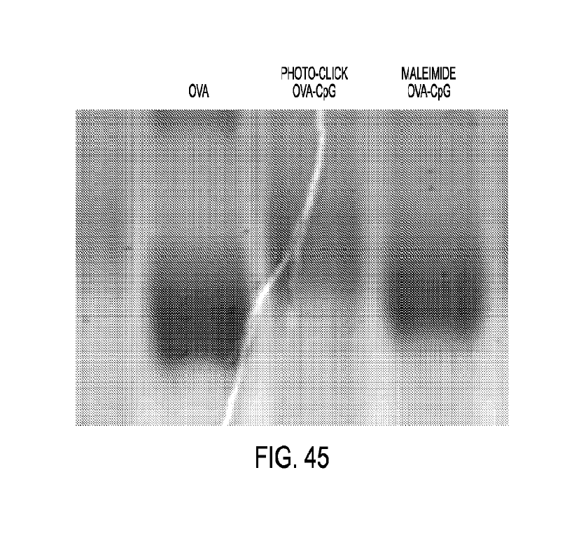

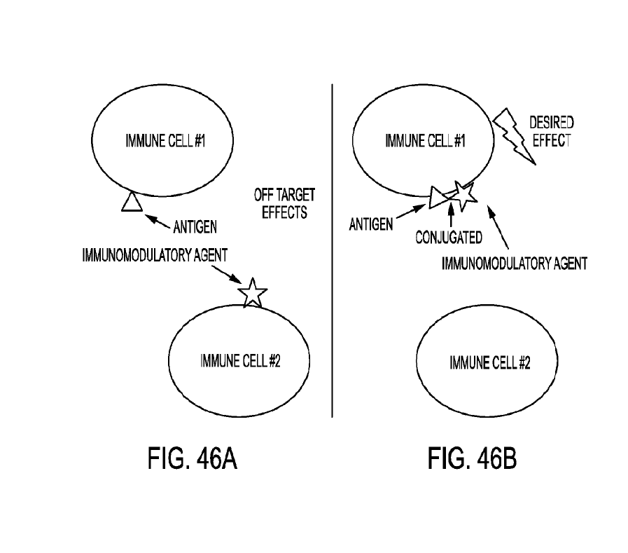

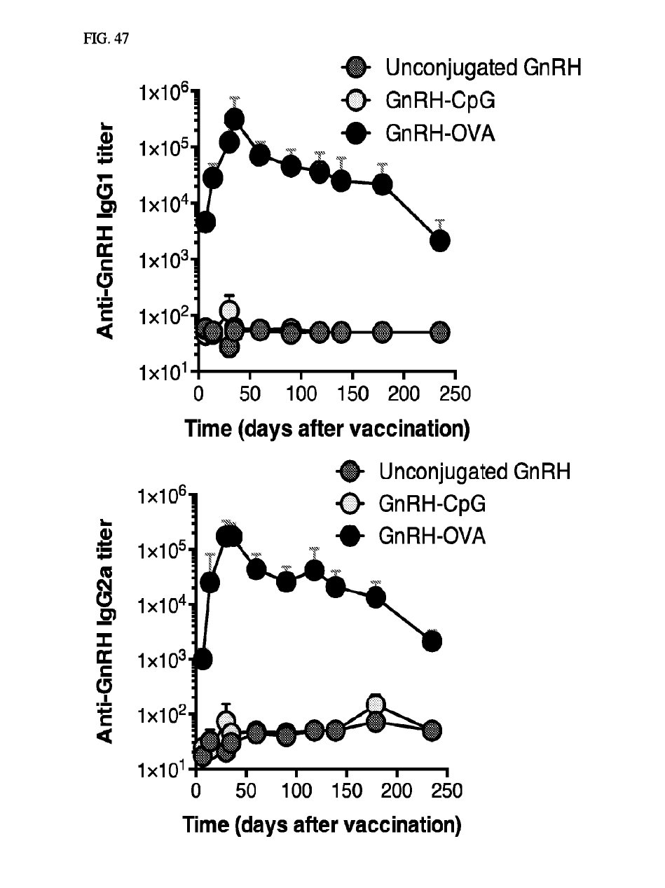

The CpG-OVA conjugate was confirmed using gel electrophoresis (non-reducing, denaturing 10% Tris-Glycine). Photo-Click OVA-CpG conjugate (lane 2) resulted in more efficient conjugation. On average, Photo-Click OVA-CpG had 1 more CpG molecule per OVA protein compared to Maleimide OVA-CpG conjugate (lane 3) (FIG. 45).

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A composition comprising a delivery vehicle comprising an immunoconjugate, wherein the immunoconjugate comprises an immunomodulatory agent covalently linked to an antigen, wherein said antigen comprises a tumor antigen or an antigen from a pathogen.

2. The composition of claim 1, wherein said immunomodulatory agent comprises an adjuvant or a carrier protein.

3. The composition of claim 2, wherein
(a) said adjuvant comprises a TLR agonist or ligand;
(b) said adjuvant comprises a TLR agonist or ligand, wherein said TLR agonist or ligand comprises a CpG oligonucleotide or a poly I:C poly nucleotide; or
(c) said adjuvant comprises a TLR agonist or ligand, wherein said TLR agonist or ligand comprises a CpG oligonucleotide or a poly I:C poly nucleotide, and wherein said CpG or said poly I:C are condensed;
(d) the carrier protein is a non-tumor antigen; or
(e) the carrier protein is a non-tumor antigen, wherein the non-tumor antigen is a ovalbumin or Keyhole limpet hemocyanin.

4. (canceled)

5. (canceled)

6. (canceled)

7. (canceled)

8. (canceled)

9. The composition of claim 1, wherein said immunomodulatory agent comprises mesoporous silica.

10. The composition of claim 1, wherein said adjuvant comprises a Stimulator of Interferon Gene (STING) agonist or ligand.

11. The composition of claim 1, wherein said tumor antigen comprises
(a) a tumor cell lysate, or
(b) a central nervous system (CNS) cancer antigen, CNS Germ Cell tumor antigen, lung cancer antigen, Leukemia antigen, Multiple Myeloma antigen, Renal Cancer antigen, Malignant Glioma antigen, Medulloblastoma antigen, breast cancer antigen, prostate cancer antigen, ovarian cancer antigen, or Melanoma antigen.

12. The composition of claim 1, wherein antigen and said adjuvant are
(a) covalently linked; or
(b) linked via a stable maleimide (sulfhydryl-sulfhydryl), reducible maleimide (sulfhydryl-sulfhydryl), bifunctional maleimide (amine-sulfhydryl), carbodiimide (amine-carboxylic acid), or photo-click (norbornene-thiol) linker.

13. (canceled)

14. (canceled)

15. The composition of claim 1, wherein
(a) said delivery vehicle comprises a scaffold composition;
(b) said delivery vehicle comprises a scaffold composition, wherein said scaffold composition comprises a poly(d,l-lactide-co-glycolide) (PLG) polymer; or
(c) said delivery vehicle comprises a polylactic acid, polyglycolic acid, PLGA polymer, an alginate or alginate derivative, gelatin, collagen, fibrin, hyaluronic acid, a laminin rich gel, agarose, a natural and synthetic polysaccharide, a polyamino acid, a polypeptide, a polyester, a polyanhydride, a polyphosphazine, a poly(vinyl alcohol), a poly(alkylene oxide), a poly(allylamine)(PAM), a poly(acrylate), a modified styrene polymer, a pluronic polyol, a polyoxamer, a poly(uronic acid), a poly(vinylpyrrolidone), a copolymer or graft copolymer cryogel delivery scaffold or vehicles, a pore forming gel, or a mesoporous silica delivery scaffold.

16. (canceled)

17. (canceled)

18. The composition of claim 1, wherein said immunoconjugate is covalently linked to said scaffold composition, or is incorporated into, coated onto, or absorbed into said scaffold composition.

19. (canceled)

20. A method of eliciting an immune response to a tumor or a pathogen comprising administering to a subject the composition of claim 1.

21. A composition comprising an antigen or an immunoconjugate covalently linked to a scaffold composition.

22. The composition of claim 21, wherein the scaffold composition comprises mesoporous silica;

23. A composition comprising an antigen covalently linked to a tolerogen.

24. The composition of claim 23, wherein
(a) the antigen is associated with an immune activation disorder;
(b) the antigen is associated with an immune activation disorder;
(c) the antigen is associated with an immune activation disorder, wherein the antigen comprises i) a peptide associated with an immune activation disorder or ii) an antigen from a lysate of a cell associated with an immune activation disorder;
(d) the tolerogen comprises dexamethasone, vitamin D, retinoic acid, thymic stromal lymphopoietin, rapamycin, aspirin, transforming growth factor beta, interleukin-10, vasoactive intestinal peptide, vascular endothelial growth factor, retinoic acid, estrogen, anti-CTLA4 immunoglobulin, P-selectin, galectin 1, binding immunoglobulin protein (BiP), hepatocyte growth factor (HGF), immunoglobulin-like transcript 3 (ILT3), aspirin, resveratrol, rosiglitazone, curcumin, prednisolone, LF 15-0195, carvacrol, or a derivative thereof;
(e) the antigen is associated with an immune activation disorder, wherein the immune activation disorder comprises an autoimmune disorder, an allergy, asthma, transplant rejection, septic shock, and macrophage activation syndrome;
(f) the immune activation disorder comprises an autoimmune disorder;
(g) the immune activation disorder comprises an autoimmune disorder, wherein the autoimmune disorder comprises multiple sclerosis, type 1 diabetes mellitus, Crohn's disease, rheumatoid arthritis, systemic lupus erythematosus, scleroderma, alopecia areata, antiphospholipid antibody syndrome, autoimmune hepatitis, celiac disease, Graves' disease, Guillain-Barre syndrome, Hashimoto's disease, hemolytic anemia, idiopathic thrombocytopenic purpura, inflammatory bowel disease, ulcerative colitis, inflammatory myopathies, polymyositis, myasthenia gravis, primary biliary cirrhosis, psoriasis, Sjögren's syndrome, vitiligo, gout, atopic dermatitis, acne vulgaris, or autoimmune pancreatitis;
(h) the immune activation disorder comprises an autoimmune disorder, wherein the autoimmune disorder comprises type 1 diabetes;
(i) the peptide comprises a pancreatic peptide or protein;
(j) the peptide comprises a pancreatic peptide or protein, wherein the pancreatic peptide or protein comprises insulin, proinsulin, glutamic acid decarboxylase-65 (GAD65), insulinoma-associated protein 2, heat shock protein 60, ZnT8, islet-specific glucose-6-phosphatase catalytic subunit related protein (IGRP), or a fragment thereof;
(k) the autoimmune disorder comprises multiple sclerosis;
(l) the peptide comprises myelin basic protein (MBP), myelin proteolipid protein, myelin-associated oligodendrocyte basic protein, myelin oligodendrocyte glycoprotein (MOG), or a fragment thereof;
(m) the peptide comprises myelin basic protein (MBP), myelin proteolipid protein, myelin-associated oligodendrocyte basic protein, myelin oligodendrocyte glycoprotein (MOG), or a fragment thereof;
(n) the tolerogen comprises dexamethasone or a derivative thereof;
(o) the tolerogen comprises dexamethasone;
(p) the tolerogen is dexamethasone derivatized with a phosphate at the primary alcohol on carbon 21 (or the ketone hydroxyl);
(q) the tolerogen is linked to the N-terminus or C-terminus of the peptide;
(r) the lysate comprises a peptide, and wherein the tolerogen is linked to the N-terminus or C-terminus of the peptide; or
(s) the tolerogen is covalently linked to the antigen by a carbamate bond, an ester bond, an amide bond, a linker or a bond resulting from (i) Azide-Alkyne Cycloaddition, (ii) Copper-Free Azide Alkyne Cycloaddition, or (iii) Staudinger Ligation.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. The composition of claim 23, wherein the antigen comprises a peptide derived from MOG, and wherein the tolerogen comprises dexamethasone or a derivative thereof.

42. The composition of claim 41, wherein the MOG is human MOG, and wherein the peptide comprises amino acids 35-55 of the human MOG, or wherein the MOG is mouse MOG, and wherein the peptide comprises the amino acid sequence, MEVGWYRSPFSRVVHLYRNGK (SEQ ID NO: 8).

43. The composition of claim 23, further comprising a delivery vehicle and a dendritic cell recruitment composition.

44. The composition of claim 43, wherein
(a) the dendritic cell recruitment composition comprises granulocyte-macrophage colony stimulating factor (GM-CSF), FMS-like tyrosine kinase 3 ligand, N-formyl peptides, fractalkine, monocyte chemotactic protein-1, or macrophage inflammatory protein-3 (MIP-3α);
(b) further comprising a Th1 promoting agent, wherein the Th1 promoting agent comprises a toll-like receptor (TLR) agonist;
(c) the device comprises a microchip or a polymer;
(d) the device comprises a polymer;
(e) the device comprises a polymer, wherein the polymer is selected from poly(ortho ester I), poly(ortho ester) II, poly(ortho ester) III, poly(ortho ester) IV, polyanhydride, alginate, poly(ethylene glycol), hyaluronic acid, collagen, gelatin, poly (vinyl alcohol), fibrin, poly (glutamic acid), peptide amphiphiles, silk, fibronectin, chitin, poly(methyl methacrylate), poly(ethylene terephthalate), poly(dimethylsiloxane), poly(tetrafluoroethylene), polyethylene, polyurethane, poly(glycolic acid), poly(lactic acid), poly(caprolactone), poly(lactide-co-glycolide), polydioxanone, polyglyconate, BAK, polypropylene fumarate, poly[(carboxy phenoxy)propane-sebacic acid], poly[pyromellitylimidoalanine-co-1,6-bis(p-carboxy phenoxy)hexane], polyphosphazene, starch, cellulose, albumin, polyhydroxyalkanoates, Poly(lactide), and poly(glycolide);
(f) the device comprises a polymer, wherein the polymer is hydrophobic or hydrophilic;
(g) the device comprises a polymer, wherein the polymer is hydrophobic;
(h) the device comprises a polymer, wherein the polymer is hydrophobic, and wherein the polymer is a polyanhydride, a poly (ortho ester), poly (glutamic acid), peptide amphiphiles, poly(ethylene terephthalate), poly(tetrafluoroethylene), polyurethane, poly(glycolic acid), poly(lactic acid), poly(caprolactone), poly(lactide-co-glycolide), polydioxanone, polyglyconate, BAK, poly(ortho ester I), poly(ortho ester) II, poly(ortho ester) III, poly(ortho ester) IV, polypropylene fumarate, poly[(carboxy phenoxy)propane-sebacic acid],
poly[pyromellitylimidoalanine-co-1,6-bis(p-carboxyphenoxy)hexane], or polyphosphazene polyhydroxyalkanoates;
(i) comprising a poly(d,l-lactide-co-glycolide) (PLG) polymer; or
(j) comprising a polylactic acid, polyglycolic acid, PLGA polymer, an alginate or alginate derivative, gelatin, collagen, fibrin, hyaluronic acid, a laminin rich gel, agarose, a natural and synthetic polysaccharide, a polyamino acid, a polypeptide, a polyester, a polyanhydride, a polyphosphazine, a poly(vinyl alcohol), a poly(alkylene oxide), a poly(allylamine)(PAM), a poly(acrylate), a modified styrene polymer, a pluronic polyol, a polyoxamer, a poly(uronic acid), a poly(vinylpyrrolidone), a copolymer or graft copolymer cryogel delivery scaffold or vehicles, a pore forming gel, or a mesoporous silica delivery scaffold.

45. (canceled)

46. (canceled)

47. (canceled)

48. (canceled)

49. (canceled)

50. (canceled)

51. (canceled)

52. (canceled)

53. (canceled)

54. (canceled)

55. A method of reducing the severity of an autoimmune disorder in a subject in need thereof, comprising administering the composition of claim 23 to a subject suffering from an autoimmune disorder, wherein the tolerogen induces immune tolerance or a reduction in an immune response, and wherein the antigen is derived from a cell to which a pathologic autoimmune response associated with the autoimmune disorder is directed.

56. The method of claim 55, wherein
(a) the autoimmune disorder comprises multiple sclerosis, type 1 diabetes mellitus, Crohn's disease, rheumatoid arthritis, systemic lupus erythematosus, scleroderma, alopecia areata, antiphospholipid antibody syndrome, autoimmune hepatitis, celiac disease, Graves' disease, Guillain-Barre syndrome, Hashimoto's disease, hemolytic anemia, idiopathic thrombocytopenic purpura, inflammatory bowel disease, ulcerative colitis, inflammatory myopathies, polymyositis, myasthenia gravis, primary biliary cirrhosis, psoriasis, Sjögren's syndrome, vitiligo, gout, atopic dermatitis, acne vulgaris, or autoimmune pancreatitis; or
(b) the autoimmune disorder is multiple sclerosis.

57. (canceled)

58. A method of (a) reducing the severity of an allergy in a subject in need thereof, comprising administering the composition of claim 23 to a subject suffering from an allergy, wherein the antigen is associated with the allergy; or (b) reducing the severity or frequency of an asthmatic attack in a subject in need thereof, comprising administering the composition of claim 1 to a subject suffering from or at risk for an asthmatic attack, wherein the antigen provokes the asthmatic attack.

59. The method of claim 58, wherein
(a) the antigen comprises an allergen;
(b) the antigen comprises an allergen, wherein said allergen comprises (Amb a 1 (ragweed allergen), Der p2 (Dermatophagoides pteronyssinus allergen, the main species of house dust mite and a major inducer of asthma), Betv 1 (major White Birch (Betula verrucosa) pollen antigen), Aln g I from Alnus glutinosa (alder), Api G I from Apium graveolens (celery), Car b I from Carpinus betulus (European hornbeam), Cor a I from Corylus avellana (European hazel), Mal d I from Malus domestica (apple), phospholipase A2 (bee venom), hyaluronidase (bee venom), allergen C (bee venom), Api m 6 (bee venom), Fel d 1 (cat), Fel d 4 (cat), Gal d 1 (egg), ovotransferrin (egg), lysozyme (egg), ovalbumin (egg), casein (milk) and whey proteins (alpha-lactalbumin and beta-lactaglobulin, milk), Ara h 1 through Ara h 8 (peanut), vicilin (tree nut), legumin (tree nut), 2S albumin (tree nut), profilins, heveins, lipid transfer proteins, Cor a 1 (hazelnut), Cor a 1.01 (hazel pollen), Cor a 1.02 (hazel pollen), Cor a 1.03 (hazel pollen), Cor a 1.04 (hazelnut), Bet v 1 (hazelnut), Cor a 2 (hazelnut), glycinin (soybean), Cor a 11 (hazelnut), Cor a 8 (tree nut), rJug r 1 (walnut), rJug r 2 (walnut), Jug r 3 (walnut), Jug r 4 (walnut), Ana o 1 (cashew nut), Ana o 2 (cashew nut), Cas s 5 (chestnut), Cas s 8 (chestnut), Ber e 1 (Brazil nut), Mal d 3 (apple), or Pru p 3 (peach).

60. (canceled)

61. (canceled)

62. (canceled)

63. A method of reducing transplant rejection in a subject in need thereof, comprising administering the composition of claim 21 to a subject prior to, during, or after a cell or tissue transplantation procedure, wherein the antigen comprises a molecule present in the transplanted cell but not present in the subject prior to the transplantation procedure.

64. The method of claim 63, wherein
(a) the antigen comprises an alloantigen;
(b) the antigen comprises a minor or major histocompatibility antigen; or
(c) the antigen comprises a minor or major histocompatibility antigen, wherein the antigen comprises a major histocompatibility complex (MHC) molecule, a HLA class I molecule, or a minor H antigen.

65. (canceled)

66. (canceled)

67. A scaffold composition comprising an antigen, a recruitment composition,
and a tolerogen.

68. The composition of claim 67,
(a) further comprising a Th1 promoting agent;
(b) wherein said tolerogen comprises thymic stromal lymphopoietin, dexamethasone, vitamin D, retinoic acid, rapamycin, aspirin, transforming growth factor beta, interleukin-10, vasoactive intestinal peptide, or vascular endothelial growth factor;
(c) wherein said recruitment composition comprises GM-CSF, FMS-like tyrosine kinase 3 ligand, N-formyl peptides, fractalkine, or monocyte chemotactic protein-1;
(d) further comprising a Th1 promoting agent, wherein said Th1 promoting agent comprises a toll-like receptor (TLR) agonist;
(e) further comprising a Th1 promoting agent, wherein said Th1 promoting agent comprises a toll-like receptor (TLR) agonist, and wherein said TLR agonist comprises CpG;
(f) further comprising a Th1 promoting agent, wherein said Th1 promoting agent comprises a pathogen-associated molecular pattern composition or an alarmin;
(g) further comprising a Th1 promoting agent, wherein said Th1 promoting agent comprises a TLR 3, 4, or 7 agonist; or
(h) wherein said antigen comprises an autoantigen.

69. (canceled)

70. (canceled)

71. (canceled)

72. (canceled)

73. (canceled)

74. (canceled)

75. (canceled)

76. (canceled)

77. A scaffold composition comprising an allergen, a recruitment composition, and a Th1-promoting adjuvant.

78. A method of preferentially directing a Th1-mediated antigen-specific immune response, comprising administering to a subject a gel scaffold comprising an antigen, a recruitment composition and a tolerogen, wherein a dendritic cell or Treg cell is recruited to said scaffold, exposed to said antigen, and migrates away from said scaffold into a tissue of said subject and wherein said Th1 immune response is preferentially generated compared to a Th2 immune response.

79. The method of claim 77, wherein said scaffold further comprises a Th1 promoting agent.

80. A method of reducing the severity of an autoimmune disorder, comprising identifying a subject suffering from an autoimmune disorder and administering to said subject the scaffold composition of claim 67, wherein said antigen is derived from a cell to which a pathologic autoimmune response associated with said disorder is directed.

81. The method of claim 79, wherein
(a) said autoimmune disorder is type 1 diabetes and said antigen comprises a pancreatic cell antigen;
(b) said autoimmune disorder is type 1 diabetes and said antigen comprises a pancreatic cell antigen, wherein said antigen comprises insulin, proinsulin, glutamic acid decarboxylase-65 (GAD65), insulinoma-associated protein 2, heat shock protein 60, ZnT8, or islet-specific glucose-6-phosphatase catalytic subunit;
(c) said autoimmune disorder is multiple sclerosis; and
(d) said autoimmune disorder is multiple sclerosis, wherein said antigen comprises myelin basic protein myelin basic protein, myelin proteolipid protein, myelin-associated oligodendrocyte basic protein, or myelin oligodendrocyte glycoprotein.

82. (canceled)

83. (canceled)

84. A method of reducing the severity of an chronic inflammatory disorder or allergy, comprising identifying a subject suffering from said chronic inflammatory disorder or allergy and administering to said subject a scaffold composition comprising an antigen associated with said disorder or allergy, a recruitment composition, and a Th1-promoting adjuvant.

85. A method of reducing the severity of a chronic inflammatory disorder or allergy, comprising identifying a subject suffering from said chronic inflammatory disorder or allergy and administering to said subject a scaffold composition comprising an antigen associated with said disorder or allergy, a recruitment composition, and an adjuvant.

86. The method of claim 84, wherein
(a) said antigen comprises an allergen; or
(b) said antigen comprises an allergen, wherein said allergen comprises (Amb a 1 (ragweed allergen), Der p2 (Dermatophagoides pteronyssinus allergen, the main species of house dust mite and a major inducer of asthma), Betv 1 (major White Birch (Betula verrucosa) pollen antigen), Aln g I from Alnus glutinosa (alder), Api G I from Apium graveolens (celery), Car b I from Carpinus betulus (European hornbeam), Cor a I from Corylus avellana (European hazel), Mal d I from Malus domestica (apple), phospholipase A2 (bee venom), hyaluronidase (bee venom), allergen C (bee venom), Api m 6 (bee venom), Fel d 1 (cat), Fel d 4 (cat), Gal d 1 (egg), ovotransferrin (egg), lysozyme (egg), ovalbumin (egg), casein (milk) and whey proteins (alpha-lactalbumin and beta-lactaglobulin, milk), or Ara h 1 through Ara h 8 (peanut).

87. (canceled)

88. (canceled)

89. A method of reducing inflammation in periodontal disease comprising administering to a subject the composition of claim 43, wherein said composition recruits and programs dendritic cells to be tolerogenic, wherein the tolerogenic dendritic cells promote regulatory T-cell differentiation, leading to formation of regulatory T-cells, decreased effector T-cells, and a reduction in periodontal inflammation.

90. The method of claim 88, wherein the tolerogenic dendritic cells migrate from the delivery vehicle to lymph nodes.

91. A biomaterial system that decreases inflammation and increases bone regeneration for use in a subject afflicted with periodontitis, comprising a plasmid DNA that encodes BMP-2, wherein the biomaterial system delivers the plasmid DNA to a dendritic cell, thereby suppressing inflammation and increasing bone regeneration.

92. The biomaterial of claim 90, wherein (a) the bone regeneration is alveolar bone regeneration; or (b) the bone occurs at the site of periodontitis in the subject.

93. (canceled)

94. (canceled)


SEQUENCE LISTING (30 sequences)

SEQ ID NO: 1 - protein, 15 aa, Artificial Sequence
  Exemplary Peptide B9-23
SHLVEALYLVCGERG

SEQ ID NO: 2 - protein, 9 aa, Artificial Sequence
  Exemplary Peptide CP and C1
GLRILLLKV

SEQ ID NO: 3 - protein, 24 aa, Artificial Sequence
  P277 Residues 437-460 in the H-HSP65 Sequence
VLGGGCALLRCIPALDSLTPANED

SEQ ID NO: 4 - protein, 4 aa, Artificial Sequence
  Exemplary Peptide
YFAK

SEQ ID NO: 5 - protein, 15 aa, Artificial Sequence
  Exemplary Peptide
EKPKFEAYKAAAAPA

SEQ ID NO: 6 - protein, 15 aa, Artificial Sequence
  Exemplary Peptide
EKPKVEAYKAAAAPA

SEQ ID NO: 7 - protein, 15 aa, Artificial Sequence
  Exemplary Peptide
EKPKYEAYKAAAAPA

SEQ ID NO: 8 - protein, 21 aa, Artificial Sequence
  Amino Acids 35-55 of the mouse MOG
MEVGWYRSPFSRVVHLYRNGK

SEQ ID NO: 9 - protein, 8 aa, Artificial Sequence
  Exemplary ovalbumin peptide.
SIINFEKL

SEQ ID NO: 10 - protein, 128 aa, Artificial Sequence
  Human Recombinant GM-CSF
MAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 11 - protein, 125 aa, Artificial Sequence
  Murine Recombinant GM-CSF
MAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQK

SEQ ID NO: 12 - DNA, 781 bp, Homo sapiens
acacagagagaaaggctaaagttctctggaggatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtgagaccggccagatgaggctggccaagccggggagctgctctctcatgaaacaagagctagaaactcaggatggtcatcttggagggaccaaggggtgggccacagccatggtgggagtggcctggacctgccctgggccacactgaccctgatacaggcatggcagaagaatgggaatattttatactgacagaaatcagtaatatttatatatttatatttttaaaatatttatttatttatttatttaagttcatattccatatttattcaagatgttttaccgtaataattattattaaaaatatgcttcta

SEQ ID NO: 13 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 14 - protein, 235 aa, Homo sapiens
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 15 - protein, 11 aa, Artificial Sequence
  TAT 47-57 peptide
YGRKKRRQRRR

SEQ ID NO: 16 - RNA, 10 nt, Artificial Sequence
  bacterial ribosomal RNA sequence
cggaaagacc

SEQ ID NO: 17 - protein, 9 aa, Artificial Sequence
  Exemplary Peptide
SVYDFFVWL

SEQ ID NO: 18 - protein, 9 aa, Artificial Sequence
  Ovalbumin CD8 epitope
CSIINFEKL

SEQ ID NO: 19 - protein, 18 aa, Artificial Sequence
  Ovalbumin CD4 epitope
CISQAVHAAHAEINEAGR

SEQ ID NO: 20 - protein, 17 aa, Artificial Sequence
  Ovalbumin CD4 epitope
ISQAVHAAHAEINEAGR

SEQ ID NO: 21 - DNA, 20 bp, Artificial Sequence
  ODN 1585
ggggtcaacgttgagggggg

SEQ ID NO: 22 - DNA, 20 bp, Artificial Sequence
  ODN 1668
tccatgacgttcctgatgct

SEQ ID NO: 23 - DNA, 20 bp, Artificial Sequence
  ODN 1826
tccatgacgttcctgacgtt

SEQ ID NO: 24 - DNA, 24 bp, Artificial Sequence
  ODN 2006
tcgtcgttttgtcgttttgtcgtt

SEQ ID NO: 25 - DNA, 29 bp, Artificial Sequence
  ODN 2006-G5
tcgtcgttttgtcgttttgtcgttggggg

SEQ ID NO: 26 - DNA, 20 bp, Artificial Sequence
  ODN 2216
gggggacgatcgtcgggggg

SEQ ID NO: 27 - DNA, 21 bp, Artificial Sequence
  ODN 2336
ggggacgacgtcgtggggggg

SEQ ID NO: 28 - DNA, 22 bp, Artificial Sequence
  ODN 2395
tcgtcgttttcggcgcgcgccg

SEQ ID NO: 29 - DNA, 25 bp, Artificial Sequence
  ODN M362
tcgtcgtcgttcgaacgacgttgat

SEQ ID NO: 30 - protein, 17 aa, Homo sapiens
MWLQSLLLLGTVACSIS